SEQUENCE LISTING (120 sequences)

SEQ ID NO: 1 - DNA, 39 bp, Artificial Sequence
  Description of Artificial Sequence Synthetic oligonucleotide
actgctggatcgtatgcgtagtctggacgtctaccgtgt

SEQ ID NO: 2 - DNA, 26 bp, Artificial Sequence
  Description of Artificial Sequence Synthetic oligonucleotide
cagtgcaggctacgcataccatccag

SEQ ID NO: 3 - DNA, 26 bp, Artificial Sequence
  Description of Artificial Sequence Synthetic oligonucleotide
actgacacggtagacgtccagcctgc

SEQ ID NO: 4 - DNA, 39 bp, Artificial Sequence; identical to SEQ ID NO: 1
  Description of Artificial Sequence Synthetic oligonucleotide

SEQ ID NO: 5 - DNA, 26 bp, Artificial Sequence
  Description of Artificial Sequence Synthetic oligonucleotide
cagtgcaggctacgcataccatccag

SEQ ID NO: 6 - DNA, 26 bp, Artificial Sequence
  Description of Artificial Sequence Synthetic oligonucleotide
actgacacggtagacgtccagcctgc

SEQ ID NO: 7 - DNA, 4 bp, Artificial Sequence
  Description of Artificial Sequence Synthetic oligonucleotide
actg

SEQ ID NO: 8 - DNA, 4 bp, Artificial Sequence
  Description of Artificial Sequence Synthetic oligonucleotide
cagt

SEQ ID NO: 9 - DNA, 16 bp, Artificial Sequence
  Description of Artificial Sequence Synthetic oligonucleotide
ctggatcgtatgcgta

SEQ ID NO: 10 - DNA, 7 bp, Artificial Sequence
  Description of Artificial Sequence Synthetic oligonucleotide
gcaggct

SEQ ID NO: 11 - DNA, 16 bp, Artificial Sequence
  Description of Artificial Sequence Synthetic oligonucleotide
acacggtagacgtcca

SEQ ID NO: 12 - DNA, 3 bp, Artificial Sequence
  Description of Artificial Sequence Synthetic oligonucleotide
gtc

SEQ ID NO: 13 - DNA, 16 bp, Artificial Sequence
  Description of Artificial Sequence Synthetic oligonucleotide
tggacgtctaccgtgt

SEQ ID NO: 14 - DNA, 15 bp, Artificial Sequence
  Description of Artificial Sequence Synthetic oligonucleotide
acgcataccatccag

SEQ ID NO: 15 - DNA, 6 bp, Artificial Sequence
  Description of Artificial Sequence Synthetic oligonucleotide
gcctgc

SEQ ID NO: 16 - DNA, 30 bp, Artificial Sequence
  Description of Artificial Sequence Synthetic oligonucleotide
ctgagtacctgattggcttagtcggaagct

SEQ ID NO: 17 - DNA, 30 bp, Artificial Sequence
  Description of Artificial Sequence Synthetic oligonucleotide
acctaggcgtactatcaggtactcagttaa

SEQ ID NO: 18 - DNA, 30 bp, Artificial Sequence
  Description of Artificial Sequence Synthetic oligonucleotide
gaatgccgcttacagtacgcctaggttcga

SEQ ID NO: 19 - DNA, 30 bp, Artificial Sequence
  Description of Artificial Sequence Synthetic oligonucleotide
tccgactaagccagtaagcggcattcctag

SEQ ID NO: 20 - DNA, 4 bp, Artificial Sequence
  Description of Artificial Sequence Synthetic oligonucleotide
agct

SEQ ID NO: 21 - DNA, 4 bp, Artificial Sequence
  Description of Artificial Sequence Synthetic oligonucleotide
ttaa

SEQ ID NO: 22 - DNA, 4 bp, Artificial Sequence
  Description of Artificial Sequence Synthetic oligonucleotide
tcga

SEQ ID NO: 23 - DNA, 4 bp, Artificial Sequence
  Description of Artificial Sequence Synthetic oligonucleotide
ctag

SEQ ID NO: 24 - DNA, 13 bp, Artificial Sequence
  Description of Artificial Sequence Synthetic oligonucleotide
tggcttagtcgga

SEQ ID NO: 25 - DNA, 13 bp, Artificial Sequence
  Description of Artificial Sequence Synthetic oligonucleotide
atcaggtactcag

SEQ ID NO: 26 - DNA, 13 bp, Artificial Sequence
  Description of Artificial Sequence Synthetic oligonucleotide
agtacgcctaggt

SEQ ID NO: 27 - DNA, 13 bp, Artificial Sequence
  Description of Artificial Sequence Synthetic oligonucleotide
gtaagcggcattc

SEQ ID NO: 28 - DNA, 13 bp, Artificial Sequence
  Description of Artificial Sequence Synthetic oligonucleotide
ctgagtacctgat

SEQ ID NO: 29 - DNA, 13 bp, Artificial Sequence
  Description of Artificial Sequence Synthetic oligonucleotide
acctaggcgtact

SEQ ID NO: 30 - DNA, 13 bp, Artificial Sequence
  Description of Artificial Sequence Synthetic oligonucleotide
gaatgccgcttac

SEQ ID NO: 31 - DNA, 13 bp, Artificial Sequence
  Description of Artificial Sequence Synthetic oligonucleotide
tccgactaagcca

SEQ ID NO: 32 - DNA, 30 bp, Artificial Sequence
  Description of Artificial Sequence Synthetic oligonucleotide
tgactggatccgcatgacattcgccgtaag

SEQ ID NO: 33 - DNA, 30 bp, Artificial Sequence
  Description of Artificial Sequence Synthetic oligonucleotide
gtcatggatccgcatgacattcgccgtaag

SEQ ID NO: 34 - DNA, 30 bp, Artificial Sequence
  Description of Artificial Sequence Synthetic oligonucleotide
atcgtggatccgcatgacattcgccgtaag

SEQ ID NO: 35 - DNA, 30 bp, Artificial Sequence
  Description of Artificial Sequence Synthetic oligonucleotide
atgctggatccgcatgacattcgccgtaag

SEQ ID NO: 36 - DNA, 30 bp, Artificial Sequence
  Description of Artificial Sequence Synthetic oligonucleotide
gcaatggatccgcatgacattcgccgtaag

SEQ ID NO: 37 - DNA, 30 bp, Artificial Sequence
  Description of Artificial Sequence Synthetic oligonucleotide
tgaccttacggcgaatgaccgaatcagcct

SEQ ID NO: 38 - DNA, 30 bp, Artificial Sequence
  Description of Artificial Sequence Synthetic oligonucleotide
cgatcttacggcgaatgaccgaatcagcct

SEQ ID NO: 39 - DNA, 30 bp, Artificial Sequence
  Description of Artificial Sequence Synthetic oligonucleotide
gcatcttacggcgaatgaccgaatcagcct

SEQ ID NO: 40 - DNA, 30 bp, Artificial Sequence
  Description of Artificial Sequence Synthetic oligonucleotide
ttgccttacggcgaatgaccgaatcagcct

SEQ ID NO: 41 - DNA, 30 bp, Artificial Sequence
  Description of Artificial Sequence Synthetic oligonucleotide
ggatcttacggcgaatgaccgaatcagcct

SEQ ID NO: 42 - DNA, 30 bp, Artificial Sequence
  Description of Artificial Sequence Synthetic oligonucleotide
tgacaggctgattcggttcatgcggatcca

SEQ ID NO: 43 - DNA, 30 bp, Artificial Sequence
  Description of Artificial Sequence Synthetic oligonucleotide
cgataggctgattcggttcatgcggatcca

SEQ ID NO: 44 - DNA, 30 bp, Artificial Sequence
  Description of Artificial Sequence Synthetic oligonucleotide
gcataggctgattcggttcatgcggatcca

SEQ ID NO: 45 - DNA, 30 bp, Artificial Sequence
  Description of Artificial Sequence Synthetic oligonucleotide
ttgcaggctgattcggttcatgcggatcca

SEQ ID NO: 46 - DNA, 30 bp, Artificial Sequence
  Description of Artificial Sequence Synthetic oligonucleotide
ggataggctgattcggttcatgcggatcca

SEQ ID NO: 47 - DNA, 4 bp, Artificial Sequence
  Description of Artificial Sequence Synthetic oligonucleotide
tgac

SEQ ID NO: 48 - DNA, 4 bp, Artificial Sequence
  Description of Artificial Sequence Synthetic oligonucleotide
gtca

SEQ ID NO: 49 - DNA, 4 bp, Artificial Sequence
  Description of Artificial Sequence Synthetic oligonucleotide
cgat

SEQ ID NO: 50 - DNA, 4 bp, Artificial Sequence
  Description of Artificial Sequence Synthetic oligonucleotide
atcg

SEQ ID NO: 51 - DNA, 4 bp, Artificial Sequence
  Description of Artificial Sequence Synthetic oligonucleotide
gcat

SEQ ID NO: 52 - DNA, 4 bp, Artificial Sequence
  Description of Artificial Sequence Synthetic oligonucleotide
atgc

SEQ ID NO: 53 - DNA, 4 bp, Artificial Sequence
  Description of Artificial Sequence Synthetic oligonucleotide
ttgc

SEQ ID NO: 54 - DNA, 4 bp, Artificial Sequence
  Description of Artificial Sequence Synthetic oligonucleotide
gcaa

SEQ ID NO: 55 - DNA, 4 bp, Artificial Sequence
  Description of Artificial Sequence Synthetic oligonucleotide
ggat

SEQ ID NO: 56 - DNA, 13 bp, Artificial Sequence
  Description of Artificial Sequence Synthetic oligonucleotide
tggatccgcatga

SEQ ID NO: 57 - DNA, 13 bp, Artificial Sequence
  Description of Artificial Sequence Synthetic oligonucleotide
cattcgccgtaag

SEQ ID NO: 58 - DNA, 13 bp, Artificial Sequence
  Description of Artificial Sequence Synthetic oligonucleotide
cttacggcgaatg

SEQ ID NO: 59 - DNA, 13 bp, Artificial Sequence
  Description of Artificial Sequence Synthetic oligonucleotide
accgaatcagcct

SEQ ID NO: 60 - DNA, 13 bp, Artificial Sequence
  Description of Artificial Sequence Synthetic oligonucleotide
aggctgattcggt

SEQ ID NO: 61 - DNA, 13 bp, Artificial Sequence
  Description of Artificial Sequence Synthetic oligonucleotide
tcatgcggatcca

SEQ ID NO: 62 - DNA, 30 bp, Artificial Sequence
  Description of Artificial Sequence Synthetic oligonucleotide
ttgctggatccgcatgacattcgccgtaag

SEQ ID NO: 63 - DNA, 30 bp, Artificial Sequence
  Description of Artificial Sequence Synthetic oligonucleotide
cgtttggatccgcatgacattcgccgtaag

SEQ ID NO: 64 - DNA, 30 bp, Artificial Sequence; identical to SEQ ID NO: 35
  Description of Artificial Sequence Synthetic oligonucleotide

SEQ ID NO: 65 - DNA, 26 bp, Artificial Sequence
  Description of Artificial Sequence Synthetic oligonucleotide
tggatccgcatgacattcgccgtaag

SEQ ID NO: 66 - DNA, 30 bp, Artificial Sequence; identical to SEQ ID NO: 39
  Description of Artificial Sequence Synthetic oligonucleotide

SEQ ID NO: 67 - DNA, 30 bp, Artificial Sequence
  Description of Artificial Sequence Synthetic oligonucleotide
gcaacttacggcgaatgaccgaatcagcct

SEQ ID NO: 68 - DNA, 26 bp, Artificial Sequence
  Description of Artificial Sequence Synthetic oligonucleotide
cttacggcgaatgaccgaatcagcct

SEQ ID NO: 69 - DNA, 30 bp, Artificial Sequence; identical to SEQ ID NO: 44
  Description of Artificial Sequence Synthetic oligonucleotide

SEQ ID NO: 70 - DNA, 30 bp, Artificial Sequence; identical to SEQ ID NO: 45
  Description of Artificial Sequence Synthetic oligonucleotide

SEQ ID NO: 71 - DNA, 30 bp, Artificial Sequence
  Description of Artificial Sequence Synthetic oligonucleotide
aacgaggctgattcggttcatgcggatcca

SEQ ID NO: 72 - DNA, 26 bp, Artificial Sequence
  Description of Artificial Sequence Synthetic oligonucleotide
aggctgattcggttcatgcggatcca

SEQ ID NO: 73 - DNA, 40 bp, Artificial Sequence
  Description of Artificial Sequence Synthetic oligonucleotide
acgtcgaccgatgaatagcggtcagatccgtacctactcg

SEQ ID NO: 74 - DNA, 40 bp, Artificial Sequence
  Description of Artificial Sequence Synthetic oligonucleotide
acgtcgagtaggtacggatctgcgtattgcgaacgactcg

SEQ ID NO: 75 - DNA, 40 bp, Artificial Sequence
  Description of Artificial Sequence Synthetic oligonucleotide
acgtcgagtcgttcgcaatacggctgtacgtatggtctcg

SEQ ID NO: 76 - DNA, 40 bp, Artificial Sequence
  Description of Artificial Sequence Synthetic oligonucleotide
acgtcgagaccatacgtacagcaccgctattcatcggtcg

SEQ ID NO: 77 - DNA, 40 bp, Artificial Sequence; identical to SEQ ID NO: 73
  Description of Artificial Sequence Synthetic oligonucleotide

SEQ ID NO: 78 - DNA, 40 bp, Artificial Sequence
  Description of Artificial Sequence Synthetic oligonucleotide
acgtcgagtcgttcgcaatacgaccgctattcatcggtcg

SEQ ID NO: 79 - DNA, 40 bp, Artificial Sequence; identical to SEQ ID NO: 74
  Description of Artificial Sequence Synthetic oligonucleotide

SEQ ID NO: 80 - DNA, 40 bp, Artificial Sequence
  Description of Artificial Sequence Synthetic oligonucleotide
acgtcgacagctgactagagtcacgacctgtacctactcg

SEQ ID NO: 81 - DNA, 47 bp, Artificial Sequence
  Description of Artificial Sequence Synthetic oligonucleotide
acgtcgagtcgttctcaagacgtagctaggactctagtcagctgtcg

SEQ ID NO: 82 - DNA, 40 bp, Artificial Sequence
  Description of Artificial Sequence Synthetic oligonucleotide
acgtcgagtaggtacaggtcgtcgtcttgagaacgactcg

SEQ ID NO: 83 - protein, 12 aa, Semian virus 40
FKKKRKVEDPYC

SEQ ID NO: 84 - protein, 13 aa, Human immunodeficiency virus
GRKKRRQRRRPPQ

SEQ ID NO: 85 - protein, 19 aa, Artificial Sequence
  Description of Artificial Sequence Synthetic peptide
MRRAHHRRRRASHRRMRGG

SEQ ID NO: 86 - DNA, 84 bp, Artificial Sequence
  Description of Artificial Sequence Synthetic oligonucleotide
ccggataaggcgcagcggtcggctgaattcagggttcgtggcaggccagcacacttggagaccgaagcttaccggactcctaac

SEQ ID NO: 87 - DNA, 87 bp, Artificial Sequence
  Description of Artificial Sequence Synthetic oligonucleotide
tcagttaggagtccggtaagcttcggtctccaagtgtgctggcctgccacgaaccctgaattcagccgaccgctgcgccttatccgg

SEQ ID NO: 88 - DNA, 90 bp, Artificial Sequence
  Description of Artificial Sequence Synthetic oligonucleotide
acctaggcgtacttggcttagtcggaactgacctaggcgtacttggcttagtcggacagttccgactaagccagtaagcggcattctagc

SEQ ID NO: 89 - DNA, 30 bp, Artificial Sequence
  Description of Artificial Sequence Synthetic oligonucleotide
gaatgccgcttacagtacgcctaggttagc

SEQ ID NO: 90 - DNA, 90 bp, Artificial Sequence
  Description of Artificial Sequence Synthetic oligonucleotide
acctaggcgtacttggcttagtcggaactgtccgactaagccagtaagcggcattccagttccgactaagccagtaagcggcattctagc

SEQ ID NO: 91 - DNA, 30 bp, Artificial Sequence; identical to SEQ ID NO: 89
  Description of Artificial Sequence Synthetic oligonucleotide

SEQ ID NO: 92 - DNA, 90 bp, Artificial Sequence
  Description of Artificial Sequence Synthetic oligonucleotide
acctaggcgtacttggcttagtcggaactggaatgccgcttacagtacgcctaggtcagttccgactaagccagtaagcggcattctagc

SEQ ID NO: 93 - DNA, 30 bp, Artificial Sequence
  Description of Artificial Sequence Synthetic oligonucleotide
gaatgcggcttacagtacgcctaggttagc

SEQ ID NO: 94 - DNA, 30 bp, Artificial Sequence
  Description of Artificial Sequence Synthetic oligonucleotide
tctccacgctaactctgtgtcatcgtactg

SEQ ID NO: 95 - DNA, 30 bp, Artificial Sequence
  Description of Artificial Sequence Synthetic oligonucleotide
acgatgacagagaatggtcgcaagtcctag

SEQ ID NO: 96 - DNA, 29 bp, Artificial Sequence
  Description of Artificial Sequence Synthetic oligonucleotide
acttgcgaccatgttagcgtggagatcga

SEQ ID NO: 97 - DNA, 30 bp, Artificial Sequence; identical to SEQ ID NO: 16
  Description of Artificial Sequence Synthetic oligonucleotide

SEQ ID NO: 98 - DNA, 30 bp, Artificial Sequence; identical to SEQ ID NO: 17
  Description of Artificial Sequence Synthetic oligonucleotide

SEQ ID NO: 99 - DNA, 30 bp, Artificial Sequence; identical to SEQ ID NO: 18
  Description of Artificial Sequence Synthetic oligonucleotide

SEQ ID NO: 100 - DNA, 30 bp, Artificial Sequence; identical to SEQ ID NO: 19
  Description of Artificial Sequence Synthetic oligonucleotide

SEQ ID NO: 101 - DNA, 29 bp, Artificial Sequence
  Description of Artificial Sequence Synthetic oligonucleotide
acttgcgaccatgttagcgtggagatcga

SEQ ID NO: 102 - DNA, 60 bp, Artificial Sequence
  Description of Artificial Sequence Synthetic oligonucleotide
tctccacgctaactctgtgtcatcgtactgacgatgacacagaatggtcgcaagtcttaa

SEQ ID NO: 103 - DNA, 26 bp, Artificial Sequence
  Description of Artificial Sequence Synthetic oligonucleotide
gacttgcgaccatgttagcgtggaga

SEQ ID NO: 104 - DNA, 60 bp, Artificial Sequence
  Description of Artificial Sequence Synthetic oligonucleotide
tctccacgctaactctgtgtcatcgtcagtaccatgagacagaatggtcgcaagtcctag

SEQ ID NO: 105 - protein, 4 aa, Artificial Sequence
  Description of Artificial Sequence Synthetic peptide
RGDC

SEQ ID NO: 106 - protein, 4 aa, Artificial Sequence
  Description of Artificial Sequence Synthetic peptide
RGEC

SEQ ID NO: 107 - protein, 4 aa, Artificial Sequence
  Description of Artificial Sequence Synthetic peptide
RGDT

SEQ ID NO: 108 - protein, 4 aa, Artificial Sequence
  Description of Artificial Sequence Synthetic peptide
DGEA

SEQ ID NO: 109 - protein, 6 aa, Artificial Sequence
  Description of Artificial Sequence Synthetic peptide
DGEAGC

SEQ ID NO: 110 - protein, 8 aa, Artificial Sequence
  Description of Artificial Sequence Synthetic peptide
EPRGDNYR

SEQ ID NO: 111 - protein, 4 aa, Artificial Sequence
  Description of Artificial Sequence Synthetic peptide
RGDS

SEQ ID NO: 112 - protein, 5 aa, Artificial Sequence
  Description of Artificial Sequence Synthetic peptide
EILDV

SEQ ID NO: 113 - protein, 4 aa, Artificial Sequence
  Description of Artificial Sequence Synthetic peptide
REDV

SEQ ID NO: 114 - protein, 5 aa, Artificial Sequence
  Description of Artificial Sequence Synthetic peptide
YIGSR

SEQ ID NO: 115 - protein, 6 aa, Artificial Sequence
  Description of Artificial Sequence Synthetic peptide
SIKVAV

SEQ ID NO: 116 - protein, 4 aa, Artificial Sequence
  Description of Artificial Sequence Synthetic peptide
RGDV

SEQ ID NO: 117 - protein, 7 aa, Artificial Sequence
  Description of Artificial Sequence Synthetic peptide
HRNRKGV

SEQ ID NO: 118 - protein, 5 aa, Artificial Sequence
  Description of Artificial Sequence Synthetic peptide
KKGHV

SEQ ID NO: 119 - protein, 25 aa, Artificial Sequence
  Description of Artificial Sequence Synthetic peptide
XPQPNPSPASPVVVGGGASLPEFXY

SEQ ID NO: 120 - protein, 16 aa, Artificial Sequence
  Description of Artificial Sequence Synthetic peptide
ASPVVVGGGASLPEFX

(19) United States
(12) Patent Application Publication
(10) Pub. No.: US 2012/0045430 A1
(43) Pub. Date: Feb. 23, 2012

(54) DENDRIMER-LIKE MODULAR DELIVERY VECTOR

(76) Inventors: Dan Luo, Ithaca, NY (US); Yougen Li, Pennington, NJ (US)

(21) Appl. No.: 13/210,095

(22) Filed: Aug. 15, 2011

Related U.S. Application Data
(63) Continuation of application No. 11/583,990, filed on Oct. 18, 2006, now abandoned.
Application No. 11/583,990 is a continuation-in-part of application No. 11/464,181, filed on Aug. 11, 2006.
(60) Provisional application No. 60/727,961, filed on Oct. 18, 2005.
(60) Provisional application No. 60/745,383, filed on Apr. 21, 2006.
(60) Provisional application No. 60/783,426, filed on Mar. 17, 2006.
(60) Provisional application No. 60/783,422, filed on Mar. 17, 2006.
(60) Provisional application No. 60/756,453, filed on Jan. 5, 2006.
(60) Provisional application No. 60/722,032, filed on Sep. 29, 2005.
(60) Provisional application No. 60/707,431, filed on Aug. 11, 2005.

(51) Int. Cl. A61K 31/7052; A61K 38/14; A61K 38/36; A61K 39/395; A61K 38/54; A61K 38/27; A61K 38/25; A61K 38/31; A61K 38/09; A61K 38/24; A61K 38/23; A61K 38/29; A61K 38/26; A61K 38/11; A61K 38/22; A61K 38/28; A61K 39/165; A61K 39/112; A61K 39/29; A61K 39/245; A61K 39/02; A61K 39/145; A61K 39/10; A61K 39/275; A61K 39/13; A61K 39/002; A61K 39/07; A61K 39/108; A61K 39/21; A61K 35/76; A61P 35/00; A61P 29/00; A61P 31/18; A61P 37/06; A61P 25/08; A61P 31/12; A61P 25/20; A61P 25/16; A61P 21/02; A61P 33/00; A61P 33/02; A61P 31/10; A61P 9/12; A61P 7/02; A61K 39/00

(52) U.S. Cl. 424/130.1; 514/44 R; 424/184.1; 514/44 A; 514/20.9; 514/13.7; 514/14.9; 514/19.2; 514/2.3; 424/94.3; 514/11.3; 514/11.2; 514/10.3; 514/11.1; 514/9.8; 514/10.1; 514/9.9; 514/11.9; 514/11.8; 514/11.7; 514/10.9; 514/11.6; 514/12.5; 514/9.7; 514/5.9; 424/212.1; 424/258.1; 424/226.1; 424/227.1; 424/231.1; 424/236.1; 424/209.1; 424/254.1; 424/232.1; 424/217.1; 424/265.1; 424/246.1; 424/259.1; 424/208.1; 424/93.2

(57) ABSTRACT

Various nucleic acid-based matrixes are provided, comprising nucleic acid monomers as building blocks, as well as nucleic acids encoding proteins, so as to produce novel biomaterials. The nucleic acids are used to form dendrimers that are useful as supports, vectors, carriers or delivery vehicles for a variety of compounds in biomedical and biotechnological applications. In particular, the macromolecules may be used for the delivery of drugs, genetic material, imaging components or other functional molecule to which they can be conjugated. An additional feature of the macromolecules is their ability to be targeted for certain organs, tumors, or types of tissues. Methods of utilizing such biomaterials include delivery of functional molecules to cells.

### CROSS-REFERENCE TO RELATED APPLICATIONS

This application is a continuation application of U.S. Utility application Ser. No. 11/583,990, filed Oct. 18, 2006, which claims the benefit of U.S. Provisional Application Ser. No. 60/727,961, filed Oct. 18, 2005; this application is also a continuation-in-part of U.S. Utility application Ser. No. 11/464,181, filed Aug. 11, 2006, which claims the benefit of U.S. Provisional Application Ser. Nos. 60/745,383, filed Apr. 21, 2006; 60/783,426, filed Mar. 17, 2006; 60/783,422, filed Mar. 17, 2006; 60/756,453, filed Jan. 5, 2006; 60/722,032, filed Sep. 29, 2005; and 60/707,431, filed Aug. 11, 2005; the disclosures of each which are hereby incorporated by reference in their entirety.

### FIELD OF THE INVENTION

The field of the invention is nucleic acid-based polymeric structures and the use thereof.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

A key aim of biotechnology and nanotechnology is the construction of new biomaterials, including individual geometrical objects, nanomechanical devices, and extended constructions that permit the fabrication of intricate structures of materials to serve many practical purposes (Feynman et al., Miniaturization 282-296 (1961); Drexler, Proc. Nat. Acad. Sci. (USA) 78:5275-5278 (1981); Robinson et al., Prot Eng 1 295-300 (1987); Seeman, DNA & Cell Biol. 10:475-486 (1991); Seeman, Nanotechnol. 2:149-159 (1991)). Molecules of biological systems, for example, nucleic acids, have the potential to serve as building blocks for these constructions due to their self and programmable-assembly capabilities.

Nucleic acid molecules possess a distinct set of mechanical, physical, and chemical properties. From a mechanical point of view, nucleic acid molecules can be rigid (e.g., when DNA molecules are less than 50 nm, the persistent length of double stranded DNA (Bouchiat, C. et al., Biophys J 76:409-13 (1999); Tinland et al., Macromolecules 30:5763-5765 (1997); Toth et al., Biochemistry 37:8173-9 (1998)), or flexible. Physically, nucleic acid molecules are small, with a width of about 2 nanometers and a length of about 0.34 nanometers per basepair (e.g., B-DNA). In nature, nucleic acid molecules (i.e., RNA and DNA) can be found in either linear, double stranded or circular shapes. Chemically, DNA is generally stable, non-toxic, water soluble, and is commercially available in large quantities and in high purity. Unlike DNA, RNA is almost always a single-stranded molecule and has a much shorter chain of nucleotides. RNA contains ribose, rather than the deoxyribose found in DNA (there is a hydroxyl group attached to the pentose ring in the 2′ position whereas DNA has a hydrogen atom rather than a hydroxyl group). This hydroxyl group makes RNA less stable than DNA because it is more prone to hydrolysis. However, several types of RNA (tRNA, rRNA) contain a great deal of secondary structure, which help promote stability. In addition, RNA can be modified with various chemical modifiers known in the art to stabilize the molecules. Analogous molecules with modified backbones have been designed which change various characteristics of RNA, such as its instability to degradative enzymes. Some alternative antisense structural types are phosphorothioate, Morpholino, PNA (peptide nucleic acid), LNA (locked nucleic acid), TNA (treose nucleic acid) and 2′-O alkyl oligos.

Moreover, nucleic acid molecules are easily and highly manipulable by various well-known enzymes such as restriction enzymes, ligases and nucleases. Also, under proper conditions, nucleic acid molecules will self-assemble with complementary strands of nucleic acid (e.g., DNA, RNA, or Peptide Nucleic Acid, (PNA)). Furthermore, nucleic acid molecules can be amplified exponentially and ligated specifically. Thus, nucleic acid molecules are an excellent candidate for constructing nano-material and macro-material for use in biotechnology or medicine.

The concept of using nucleic acid molecules for non-genetic application has only recently emerged, such as in DNA-computation, where DNA are utilized in algorithms for solving combinatorial problems (Adleman, Science 266:1021-4 (1994); Guarnieri et al., Science 273:220-3 (1996); Ouyang et al., Science 278:446-9 (1997); Sakamoto et al., Science 288:1223-6 (2000); Benenson et al., Nature 414:430-4 (2001)), and DNA-nanotechnology, such as using DNA molecules for nano-scaled frameworks and scaffolds (Niemeyer, Applied Physics a-Materials Science & Processing 68:119-124 (1999); Seeman, Annual Review of Biophysics and Biomolecular Structure 27:225-248 (1998)). However, the design and production of DNA-based materials is still problematic (Mao et al., Nature 397:144-146 (1999); Seeman et al., Proc Natl Acad Sci USA 99:6451-6455 (2002); Yan et al., Nature 415:62-5 (2002); Mirkin et al., Nature 382:607-9 (1996); Watson et al., J Am Chem Soc 123:5592-3 (2001)). For example, nucleic acid structures are quite polydispersed with flexible arms and self-ligated circular and non-circular byproducts (Ma et al., Nucleic Acids Res 14:9745-53 (1986); Wang et al., Journal of the American Chemical Society 120:8281-8282 (1998); Nilsen et al., J Theor Biol 187:273-84 (1997)), which severely limits their utility in constructing DNA materials. Furthermore, the building blocks and motifs employed thus far are isotropic and multivalent, possibly useful for growing nano-scaled arrays and scaffolds (Winfree et al., Nature 394:539-44 (1998); Niemeyer, Applied Physics a-Materials Science & Processing 68:119-124 (1999); Seeman, Annual Review of Biophysics and Biomolecular Structure 27:225-248 (1998)), but not suitable for controlled growth, such as in dendrimers, or in creating a large quantity of monodispersed new materials, which are important to realize nucleic acid-based materials.

Other schemes of nano-construction using linear DNA molecules include a biotin-avidin based DNA network (Luo, “Novel Crosslinking Technologies to Assess Protein-DNA Binding and DNA-DNA Complexes for Gene Delivery and Expression” (Dissertation). Molecular, Cellular, and Developmental Biology Program, The Ohio State University (1997)), nanocrystals (Alivisatos et al., Nature 382:609-11 (1996)), DNA-protein nanocomplexes (Niemeyer et al., Angewandte Chemie-International Edition 37:2265-2268 (1998)), a DNA-fueled molecular machine (Yurke et al., Nature 406:605-8 (2000)), DNA-block copolymer conjugates (Watson et al., J Am Chem Soc 123:5592-3 (2001)), DNA-silver-wire (Braun et al., Nature 391:775-8 (1998)), and DNA-mediated supramolecular structures (Taton et al., Journal of the American Chemical Society 122:6305-6306 (2000)), DNA sensing via gold nanoparticles (Elghanian et al., Science 277:1078-81 (1997)), Y-shape DNA molecules (Eckardt et al., Nature 420:286 (2002)) and DNA patterning via dip-pen nanolithography (Demers et al., Science 296:1836-8 (2002)). However, the preceding prior art DNA-based structures are are further limited to linear DNA. Linear DNA was first used to construct an artificial nano-structure (Chen et al., Nature 350, 631 (1991)). Using “double crossover” DNA (two crossovers connecting two helical domains), a variety of geometric objects, periodic arrays and nanoscale mechanical devices have been constructed (Yan et al., Nature 415, 62 (2002); Yan et al., Science 301, 1882 (2003); Seeman, Trends Biochem Sci 30, 119 (2005); Pinto et al., Nano Lett 5, 2399 (2005)). Recently Lin et al. used a linear DNA molecule as a cross-linker to construct a thermal-stimulative polyacrylamide hydrogel, creating a DNA-polymer hybrid hydrogel system (Lin et al., J Biomech Eng 126, 104 (2004)).

However, dendrimer-like nucleic acid compositions have not been utilized to effect delivery of bioactive agents to cells (either in a targeted or nonspecific manner). Therefore there is a need for new biomaterials that have applications in diverse areas of biotechnology and medicine, and which provide more efficient modular delivery, sufficient release and effective cellular/tissue targeting. The present invention provides compositions and methods that provide dendrimer-like nucleic acid-based products useful in biotechnology and medicine as modular delivery vectors for a multitude of compounds.

### SUMMARY OF THE INVENTION

Certain aspects of the present invention provide a multivalent vector capable of providing a plurality of attachment points for a plurality of the same or distinct bioactive agents. Such bioactive agents include without limitation, therapeutics (e.g., drugs, nucleic acids, small organic molecules, inorganic molecules), targeting or delivery moieties (e.g., signal peptides, nucleic acid condensing peptides, antibodies, one or more receptor/ligand or other binding pair members, biotin or nucleic acids), labeling/staining moieties (e.g., quantum dots, dyes, stains, selection markers), as well as solid substrates (e.g., agarose beads, magnetic beads, etc.). Therefore, a key feature of a multivalent vector is that any number of different chemical/biochemical entities can be linked directly or indirectly to the multivalent vector. Dendrimers as described herein provide a multivalent and/or monodisperse structure that provides multiple sites for addition of one or more molecules of interest, including without limitation bioactive agents, targeting agents, selection markers, antibiotics, detection signals/labels, drugs or a combination thereof. In various embodiments, such vectors can be utilized to deliver one or more bioactive agents to a cell or animal. In other embodiments, such vectors can also be utilized in diagnostics by targeting specific cells related to disease (e.g., pathogens, cancer, etc.). Moreover, such multivalent vectors are utilized in vivo as well as in vitro.

In some aspects of the invention a composition comprises a multimer molecule, including a first, a second, and a third polynucleotide, where at least a portion of the first polynucleotide is complementary to at least a portion of the second polynucleotide, where at least a portion of the first polynucleotide is complementary to at least a portion of the third polynucleotide, where at least a portion of me second polynucleotide is complementary to at least a portion of the third polynucleotide, and where the first, second, and third polynucleotides are associated together to form a multimer, and at least one of the first, second and third polynucleotides are linked to at least one bioactive agent. In some embodiments, the multimers are trimers that are Y-shape or T-shape. In one embodiment, all the trimers are Y-shape. In another embodiment, the all the trimers are T-shape. In yet other embodiments, the trimers are Y- and T-shape.

In other aspects of the invention, a multimer molecule comprises a first, a second, a third and a fourth polynucleotide, where at least a portion of the first polynucleotide is complementary to at least a portion of the second polynucleotides, where at least a portion of the first polynucleotide is complementary to at least a portion of the fourth polynucleotide, where at least a portion of the third polynucleotide is complementary to at least a portion of second polynucleotide and where at least a portion of the third polynucleotide is complementary to at least a portion of the fourth polynucleotides, and where at least one of the first, second, third and fourth polynucleotides are linked to at least one bioactive agent. In some embodiments, the multimers are tetramers that are X-shape or dumbbell shape. For dumbbell shapes, the second polynucleotide comprises at least portions that are complementary to the first, third and fourth nucleotides. Similarly, the fourth polynucleotide comprises at least some portions that are complementary to the first, second and third polynucleotides. In one embodiment, all the tetramers are X-shape. In another embodiment, the all the tetramers are dumbbell-shape. In yet other embodiments, the tetramers are X- and dumbbell-shape.

In some aspects of the invention also provide a method of making a nucleic acid assembly by associating at least two multimers together. In some embodiments, the multimers so assembled are all of one shape (i.e., Y-, T-, X- or dumbbell shape). In yet other embodiments, the multimers so assembled are of one or more different shape. Such Y-, T-, X- or dumbbell-shape molecules are building blocks which form an assembled structure. In some embodiments, a multimer building block comprises at least one polynucleotide having a sticky end. In other embodiments, a multimer comprises polynucleotides, each of which comprises a sticky end.

In some embodiments, a nucleic acid assembly is produced by associating a plurality of multimers together. In some embodiments, such associations produce Dendrimer Like-Nucleic Acid Molecules (DL-NAMs). In yet other embodiments, DL-NAMs comprise at least some linear linker nucleic acid molecules. In various embodiments, DL-NAMs are comprised of a single shape or at least two different shape building block molecules.

In certain embodiments, the DL-NAMs are produced in a controlled fashion, by adding multimer building blocks in successive rounds to produce a highly branched, tree-shape DL-NAMs. In some embodiments, DL-NAMs are produced that are either isotropic or anisotropic, providing molecules that are linked to various other biochemical/chemical entities (e.g., therapeutics, targeting/delivery agents, labeling/staining agents, binding pair members, etc.).

In some embodiments at least on polynucleotide forming a multimer is linked to a delivery or targeting agent that is a peptide, polypeptide, a cell receptor or a receptor ligand.

In other embodiments, at least one polynucleotide forming a trimer, whether the first, second or third polynucleotides is linked to at least one bioactive agent. In yet other embodiments,

This procedure, based on using Y-DNA as building blocks, is simple and robust For example, the 4th generation of DL-DNA is close to being monodisperse, even without purification. In addition, both the Y-DNA and the DL-DNA nanoparticles are very stable. Furthermore, no self-ligated products were detected, which was commonly seen in other types of design (Ma et al., Nucleic Acids Res 14:9745-53 (1986), which is hereby incorporated by reference in its entirety). This key improvement was due to the unique design of end sequences. Thus, specifically designed polynucleotides can be combined to form Y-DNA, and specific combinations of Y-DNAs can be combined to construct DL-DNA. Both Y-DNA and DL-DNA may be 3-dimensional, and may contain branches.

In additional embodiments, DL-NAMs are utilized to form dendrimer structures that can be monodisperse and multivalent. In such embodiments, a dendrimer can be composed of a single shaped multimer or two or more different shaped multimers. Dendrimers provide a multivalent and/or monodisperse structure that provides multiple sites for addition of one or more molecules of interest, including bioactive agents, selection markers, antibiotics, detection signals/labels, drugs or a combination thereof. In various embodiments, such vectors can be utilized to deliver one or more bioactive agents to a cell or animal, including through the membrane for the cell or nucleus.

In some embodiments, the targeting or delivery agent is a peptide selected from adenovirus core peptide, a synthetic peptide, a DNA condensing peptide, a cell targeting peptide, an endosome disrupting peptide, a nuclear targeting peptide, an influenza virus HA2 peptide, a simian immunodeficiency virus gp32 peptide, an SV40 T-Ag peptide, a VP22 peptide, Adno mu peptide, SV40 NLS peptide, a Tat peptide such as HIV Tat or a Rev peptide.

In some embodiments, the multimer building blocks are ligated together.

In some embodiments, a delivery or targeting peptide is linked to one or more multimer building block by a linker molecule. Linker molecules include nucleic acid, peptide or hybrid nucleic acid-peptide molecules.

In some embodiments, the multivalent vector of the invention is linked to at least one therapeutic nucleic acid molecule, which includes a DNA vaccine, a therapeutic gene, an RNAi, an siRNA, an aptame or an antisense molecule.

### BRIEF DESCRIPTION OF THE FIGURES

The illustrations included within this specification describe many of the advantages and features of the invention. It shall be understood that similar reference numerals and characters noted within the illustrations herein may designate the same or like features of the invention. The illustrations and features depicted herein are not necessarily drawn to scale.

FIG. 1 is a schematic drawing of DNA molecular assembly. FIG. 1A depicts the assembly of Y-DNA. Three oligonucleotides were annealed together to form one Y-shape DNA, a basic building block for dendrimer-like DNA (Yₒₐ+Y_{ob}+Y_{oc}→Y₀; Y₁ₐ+Y_{1b}+Y_{1c}→Y₁; Y₂ₐ, Y_{2b}, Y_{2c}→Y₂; Y₃ₐ+Y_{3b}+Y_{3c}→Y₃; Y₄ₐ, Y_{4b}, Y_{4c}→Y₄). FIG. 1B depicts the assembly of first generation dendrimer-like DNA (G₁). The core Yo-DNA was ligated with three “Y₁”s, all with specifically designed sticky ends. The ligation was unidirectional. FIG. 1C depicts the assembly of second generation dendrimer-like DNA (G₂). G₁ DNA was ligated with six Y₂-DNAs. FIG. 1D depicts the assembly of third generation dendrimer-like DNA (G₃) and G₄.

FIG. 2A depicts an evaluation of Yo-DNA by argarose gel. Lanes 1, 2 and 3 are oligonucleotides Yₒₐ, Y_{ob}, Y_{oc}, respectively. Lanes 4, 5 and 6 correspond to the annealing products of (Yₒₐ and Y_{ob}), (Yₒₐ and Y_{oc}), and (Y_{ob} and Y_{oc}), respectively. Lanes 7, 8 and 9 correspond to the stepwise annealing products of (Yₒₐ, Y_{ob} and Y_{oc}), (Yoa Y_{oc} and Y_{ob}), and (Y_{ob}, Y_{oc} and Yₒₐ), respectively. Lane 10 corresponds to the one-pot annealing product of (Yₒₐ, Y_{ob} and Y_{oc}). FIG. 2B depicts an evaluation of Y-DNA stability. Lane 1 represents freshly made Y-DNA and lane 2 represents the same Y-DNA stored at 4° C. for 30 days.

FIG. 3 depicts the characterization of the first generation dendrimer-like DNA (G1). FIG. 3A depicts an example of sequences of G1 DL-DNA, where a Y-shape is first formed by SEQ ID NOs 88-93, and subsequently multiple of said Y-shapes are joined together. FIG. 3B is a schematic drawing of the denaturation strategy used to confirm the G1 DL-DNA structure. After G1 DL-DNA denaturation, six oligonucleotides were generated; three of these six oligonucleotides were new species with a unique length (90 bases). The remaining three were 30 bases. In FIG. 3C, lane 1 is Y-DNA and lane 2 is GI DL-DNA. In FIG. 3D, lane 1 is a molecular marker (oligonucleotide YO>>). Lane 2 is GI DL-DNA without denaturing. Lanes 3 and 4 correspond to 0.25 ug and 0.5 ug of the denatured GI DL-DNA, respectively. FIG. 3E presents an evaluation of GI DL-DNA stability. GI on Lane 1 was freshly made. GI on Lane 2 was the same as that on Lane 1 but was stored for 45 days before gel electrophoresis.

FIG. 4A is a schematic drawings of G₂ □L-DNA (left) and other higher generation DL-DNA (right). FIG. 4B depicts an evaluation of higher generation DL-DNA formation. Lanes 1, 2, 3, 4 and 5 correspond to G₁ DL-DNA, Ofe DL-DNA, G₃ DL-DNA, G₄ DL-DNA and G₅ DL-DNA, respectively.

FIG. 5 presents images of DL-DNA: AFM images of G4 DL-DNA on mica surface using standard silicon tip (lower left) and single walled carbon nanotube (SWNT) tip (top left and right), and TEM image of G₄ DL-DNA (lower right). Scale bars correspond to 100 nm.

FIG. 6 depicts divergent and convergent synthesis of a nucleic acid assembly.

FIG. 7 depicts dendrimer-like DNA.

FIG. 8 depicts a trimer in accordance with the present invention (SEQ ID NOs: 94-96).

FIG. 9 illustrates formation of an X-shape molecule.

FIG. 10 illustrates joining of several X-shape molecules.

FIG. 11 illustrates formation of a T-shape molecule.

FIG. 12 illustrates formation of several T-shape molecules into a dendrimer-like molecule.

FIG. 13 illustrates formation of matrixes comprised of different shaped molecules.

FIG. 14 illustrates X-, Y-, T-DNA building blocks.

FIG. 15 15 Shows an exemplary X-DNA (SEQ ID NOs: 97 (i.e., 5′ starting with CTGA . . . ), 98 (5′ starting with ACCT . . . ), 99 (5′ starting with GAAT . . . ) and 100 (5′ starting with TCCG . . . ).

FIG. 16 shows a dumbbell-shape DNA (SEQ ID NOs: 101-104.

FIG. 17 shows a T-shape DNA (SEQ ID NOs: 1-3)

FIG. 18 shows a dendrimer like structure comprised of nucleic acids with terminal Y-shape arms with various compounds linked to the plurality of arms.

FIG. 19 Shows a Y-shape DNA linked to various compounds, including a circular vector DNA linked to the Y-DNA via μMu component.

FIG. 20 shows a multivalent nucleic acid dendrimer for delivery into a cell, and linked to various components.

FIG. 21 depicts various schemes for solid phase assembly of DL-NMAs.

### DETAILED DESCRIPTION OF THE INVENTION

While the invention has been described with reference to the aforementioned specification, the descriptions and illustrations of the preferable embodiments herein are not meant to be construed in a limiting sense. It shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art upon reference to the present disclosure. It is therefore contemplated that the appended claims shall also cover any such modifications, variations and equivalents.

The practice of various embodiments of the invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2^{nd} edition (1989); CURRENT PROTOCOLS 1N MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

As used in the specification and claims, the singular form “a”, “an” and “the” include plural references unless the context clearly dictates otherwise. For example, the term “a cell” includes a plurality of cells, including mixtures thereof.

As used herein, the terms “biologically active agent” or “bioactive agent” are used interchangeably and include but are not limited to a biological or chemical compound such as a simple or complex organic or inorganic molecule, peptide, peptide mimetic, protein (e.g. antibody, angiogenic, anti-angiogenic and cellular growth factors), an antigen or immunogen, liposome, small interfering RNA, RNAi or a polynucleotide (e.g. vector, virus, viral vector, or anti-sense), therapeutic agents, organic or inorganic molecules can include a homogenous or heterogeneous mixture of compounds, including pharmaceuticals, radioisotopes, crude or purified plant extracts, and/or a cell, entities that alter, inhibit, activate, or otherwise affect biological or biochemical events, including classes of molecules (e.g., proteins, amino acids, peptides, polynucleotides, nucleotides, carbohydrates, sugars, lipids, nucleoproteins, glycoproteins, lipoproteins, steroids, growth factors, chemoattractants, etc.) that are commonly found in cells and tissues, whether the molecules themselves are naturally-occurring or artificially created (e.g., by synthetic or recombinant methods). Such agents may be naturally derived or synthetic.

Examples of such therapeutic bioactive agents include but are not limited to drugs, for example, anti-cancer substances, analgesics, opioids, anti-AIDS substances, anti-cancer substances, immunosuppressants (e.g., cyclosporine), anti-viral agents, enzyme inhibitors, neurotoxins, hypnotics, anti-histamines, lubricants, tranquilizers, anti-convulsants, muscle relaxants and anti-Parkinson agents, anti-spasmodics and muscle contractants including channel blockers, miotics and anti-cholinergics, anti-glaucoma compounds, anti-parasite, anti-protozoal, and/or anti-fungal compounds, modulators of cell-extracellular matrix interactions including cell growth inhibitors and anti-adhesion molecules, vasodilating agents, inhibitors of DNA, RNA or protein synthesis, anti-hypertensives, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, targeting agents, neurotransmitters, proteins, cell response modifiers, and vaccines.

Preferably, though not necessarily, the drug is one that has already been deemed safe and effective for use by the appropriate governmental agency or body. For example, drugs for human use listed by the FDA under 21 C.F.R. §§330.5, 331 through 361, and 440 through 460; drugs for veterinary use listed by the DA under 21 C.F.R. §§500 through 589, incorporated herein by reference are all considered acceptable for use in accordance with compostions and methods disclosed herein.

The terms “polynucleotide”, “nucleotide”, “nucleotide sequence”, “nucleic acid”, “nucleic acid molecule”, “nucleic acid sequence” and “oligonucleotide” are used interchangeably, and can also include plurals of each respectively depending on the context in which the terms are utilized. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA (A, B and Z structures) of any sequence, PNA, LNA, TNA (treose nucleic acid), isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components.

A polynucleotide may be further modified after polymerization, such as by conjugation with a chemical entity. The nucleic acids, used in the various embodiments disclosed herein, may be modified in a variety of ways, including by crosslinking, intra-chain modifications such as methylation and capping, and by copolymerization. Additionally, other beneficial molecules may be attached to the nucleic acid chains (i.e., bioactive agents). The nucleic acids may have naturally occurring sequences or artificial sequences. The sequence of the nucleic acid may be irrelevant for many aspects disclosed herein. However, special sequences may be used to prevent any significant effects due to the information coding properties of nucleic acids, to elicit particular cellular responses or to govern the physical structure of the molecule. A “nucleotide probe” or “probe” refers to a polynucleotide used for detecting or identifying its corresponding target polynucleotide in a hybridization reaction. The nucleic acids may comprise intron and exon sequences, modified sequences, RNA, DNA, or analogs thereof.

As used herein, the terms “isolated and/or purified” refer to in vitro preparation, isolation and/or purification of a nucleic acid molecule of the invention, so that it is not associated with in vivo substances, or is substantially purified from in vitro substances.

The following terms are used to describe the sequence relationships between two or more polynucleotides: (a) “reference sequence,” (b) “comparison window,” (c) “sequence identity,” (d) “percentage of sequence identity,” and (e) “substantial identity.” (a) As used herein, “reference sequence” is a defined sequence used as a basis for sequence comparison. A reference sequence may be a segment of or the entirety of a specified sequence.

(b) As used herein, “comparison window” makes reference to a contiguous and specified segment of a polynucleotide sequence, wherein the polynucleotide sequence in the comparison window may include additions or deletions (i.e., gaps) compared to the reference sequence (which does not include additions or deletions) for optimal alignment of the two sequences. Generally, the comparison window is at least 5, 10, or 20 contiguous nucleotides in length, and optionally can be 30, 40, 50, 100, or longer. Those of skill in the art understand that to avoid a high similarity to a reference sequence due to inclusion of gaps in the polynucleotide sequence, a gap penalty can be introduced and is subtracted from the number of matches.

Methods of alignment of sequences for comparison are well known in the art. Thus, the determination of percent identity between any two sequences can be accomplished using a mathematical algorithm. Preferred, non-limiting examples of such mathematical algorithms are the algorithm of Myers and Miller, CABIOS, 4:11 (1988), which is hereby incorporated by reference in its entirety; the local homology algorithm of Smith et al., Adv. Appl. Math., 2:482 (1981), which is hereby incorporated by reference in its entirety; the homology alignment algorithm of Needleman and Wunsch, JMB, 48:443 (1970), which is hereby incorporated by reference in its entirety; the search-for-similarity-method of Pearson and Lipman, Proc. Natl. Acad. Sci. USA, 85:2444 (1988), which is hereby incorporated by reference in its entirety; the algorithm of Karlin and Altschul, Proc. Natl. Acad. Sci. USA, 87:2264 (1990), which is hereby incorporated by reference in its entirety; modified as in Karlin and Altschul, Proc. Natl. Acad. Sci. USA, 90:5873 (1993), which is hereby incorporated by reference in its entirety.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al., Gene, 73:237 (1988), Higgins et al., CABIOS, 5:151 (1989); Corpet et al., Nucl. Acids Res., 16:10881 (1988); Huang et al., CABIOS, 8:155 (1992); and Pearson et al., Meth. Mol. Biol., 24:307 (1994), which are hereby incorporated by reference in their entirety. The ALIGN program is based on the algorithm of Myers and Miller, supra. The BLAST programs of Altschul et al., JMB, 215:403 (1990); Nucl. Acids Res., 25:3389 (1990), which are hereby incorporated by reference in their entirety, are based on the algorithm of Karlin and Altschul supra.

Software for performing BLAST analyses is publicly available through the National Center for Biotechnology Information (worldwideweb.ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues, always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al., Nucleic Acids Res. 25:3389 (1997), which is hereby incorporated by reference in its entirety. Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See worldwideweb.ncbi.nlm.nih.gov. Alignment may also be performed manually by inspection.

Comparison of nucleotide sequences for determination of percent sequence identity to the sequences disclosed herein can be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and, therefore, do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may include additions or deletions (i.e., gaps) as compared to the reference sequence (which does not include additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide includes a sequence that has at least 51%, 52%, 53%, 54%, 55%, 56%, 57%, 58%, 59%, 60%, 61%, 62%, 63%, 64%, 65%, 66%, 67%, 68%, or 69%, 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR, or the enzymatic cleavage of a polynucleotide by a ribozyme.

The term “hybridized” as applied to a polynucleotide refers to the ability of the polynucleotide to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. The hybridization reaction may constitute a step in a more extensive process, such as the initiation of a PCR reaction, or the enzymatic cleavage of a polynucleotide by a ribozyme.

As is known to one skilled in the art, hybridization can be performed under conditions of various stringency. Suitable hybridization conditions are such that the recognition interaction between the probe and target ER-stress related gene is both sufficiently specific and sufficiently stable. Conditions that increase the stringency of a hybridization reaction are widely known and published in the art. See, for example, (Sambrook, et al., (1989), supra; Nonradioactive In Situ Hybridization Application Manual, Boehringer Mannheim, second edition). The hybridization assay can be formed using probes immobilized on any solid support, including but are not limited to nitrocellulose, glass, silicon, and a variety of gene arrays. A preferred hybridization assay is conducted on high-density gene chips as described in U.S. Pat. No. 5,445,934.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution.

For DNA-DNA hybrids, the Tₘ can be approximated from the equation of Meinkoth and Wahl, Anal. Biochem., 138:267 (1984), which is hereby incorporated by reference in its entirety; Tₘ 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH.

However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ. Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry and Molecular Biology Hybridization with Nucleic Acid Probes, Part I Chapter 2 “Overview of Principles of Hybridization and the Strategy of Nucleic Acid Probe Assays,” Elsevier, N.Y. (1993), which is hereby incorporated by reference in its entirety. Generally, highly stringent hybridization and wash conditions are selected to be about 5° C. lower than the Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g. more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

For example, nucleic acids encompassed by the present invention can include nucleic acids that specifically hybridize to, or are substantially identical to, nucleic acid sequences that include any one of the sequences disclosed herein.

As used herein the term “ligation” refers to the process of joining DNA molecules together with covalent bonds. For example, DNA ligation involves creating a phosphodiester bond between the 3′ hydroxyl of one nucleotide and the 5′ phosphate of another. Ligation is preferably carried out at 4-37° C. in presence of a ligase enzyme. Suitable ligases include Thermus thermophilus ligase, Thermus acquaticus ligase, E. coli ligase, T4 ligase, and Pyrococcus ligase.

A “subject,” “individual” or “patient” is used interchangeably herein, which refers to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

In various embodiments of the invention, the nucleic acid-based matrixes can have nanoparticle, nanosphere, nanoshell, micelle, core-shell, multi-core shell, multi-layered, nanogel, microparticle, microsphere, microgel, macrogel, nanoscale, macroscale, macroscopic, block, branched, hyperbranched, hybrid, tree-like, comb-like, brush, grafting, vesicle, coil, global, coil-coil, coil-global, rod, membrane, film, coating, self-assembly, cyclic, microconduit, microchannel, nanochannel, porous, nonporous, tube, microtube, nanotube, semi-interpenetrating network, cross-linked, or a highly networked structure.

Nucleic Acid Molecules of the Invention

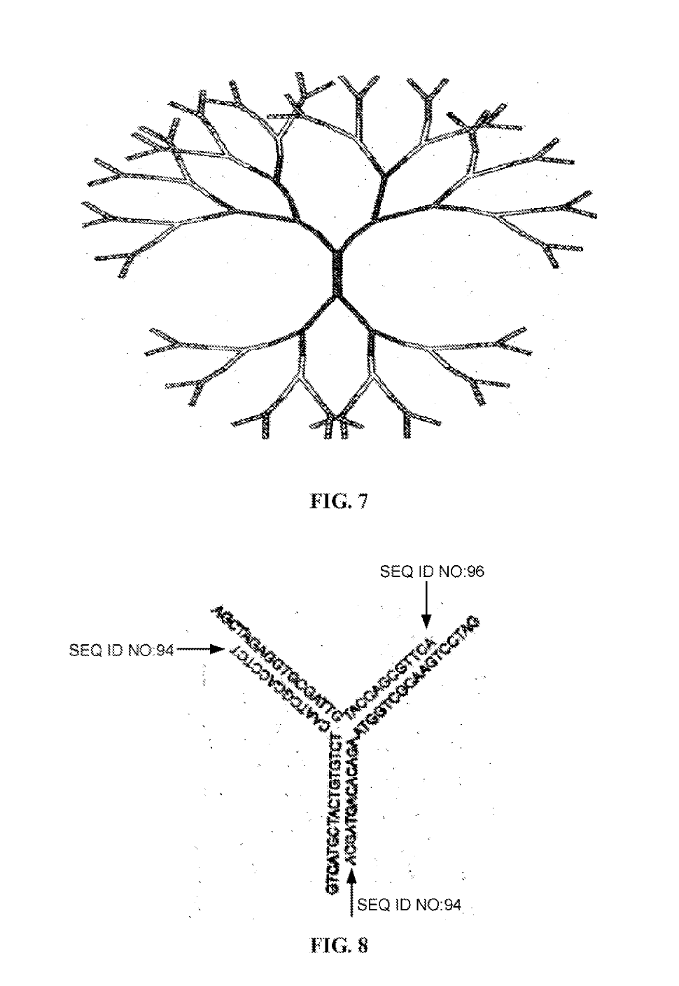

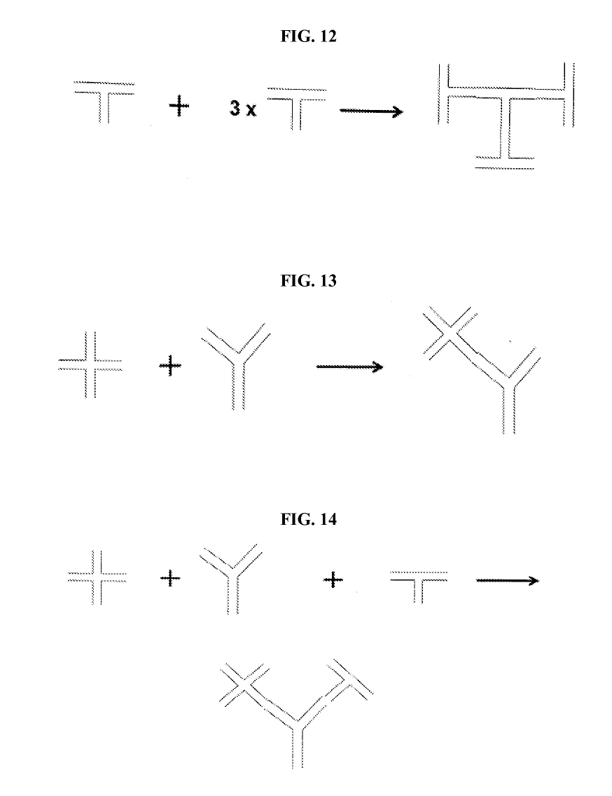

In certain aspects, the nucleic acid molecules provide monomer building blocks and/or cross-linkers that form a three-dimensional matrix or scaffold structure. A matrix of the invention can be comprised of nucleic acids that are X-shaped, Y-shaped, T-shaped, dumbbell-shaped (e.g., FIGS. 8-14) nucleic acids, or a combination thereof. Examples of various shape nucleic acids (e.g., DNA) are disclosed in U.S. patent application Ser. Nos. 10/877,697 and 60/756,453, which are incorporated by reference in their entirety. Nucleic acid building blocks are utilized for produce DL-NAMs. In one embodiment, DL-NAMs are substantially comprised of Y-shape nucleic acids. In a further embodiment, the Y-shape nucleic acid is DNA.

In other embodiments, DL-NAMs are formed of linear and branched nucleic acids. In yet further embodiments, the linear or branched nucleic acids can be DNA, RNA, PNA, TNA, LNA or any combination thereof. For example, a DL-NAMs comprise branched DNA that form building blocks supporting the dendrimer structure and also linking linear DNA that can, for example, be linked to a solid substrate.

In other embodiments, purified nucleic acids may be linked to other nucleic acids or other compounds. Linking may be accomplished in a variety of ways, including hydrogen bonds, ionic and covalent bonds, π-π bonds, polarization bonding, van der Wals forces. As used herein, “link” and “cross-link” are used interchangeably. More than one type of crosslinking may be used within a given biomaterial. Furthermore, use of a type of crosslinking easily degraded in a cell coupled with a more degradation resistant type of crosslinking may result in a biomaterial that is opened in two phases, one when the easily degraded crosslinks are broken and second when the more resistant crosslinks or the nucleic acid itself are degraded. Crosslinking may be accomplished by UV radiation, esterification, hydrolysis, intercalating agents, neoplastic agents, formaldehyde, formalin, or silica compounds. Examples of linking include but are not limited to the use of siloxane bridges as described in U.S. Pat. No. 5,214,134.

Crosslinking may occur between two strands of a double stranded nucleic acid or between the strands of two separate double strands. It may also occur between two separate single strands. Double strand to single strand crosslinking is also possible, as is crosslinking between different regions of one strand. Linkers such as small organic molecules (esters, amines) or inorganic molecules (silicas, siloxanes), including microparticles or nanoparticles thereof, may be used to attach bioactive agents to nucleic acids. Any of the different shaped nucleic acids of the invention can be linked or cross-linked by one or methods described herein. Therefore, X-shaped, Y-shaped, T-shaped, dumbbell shaped or any combination thereof can be linked to each other, as well as to other chemical moieties or polymeric compounds.

In addition, in certain aspects, where nucleic acids are linked to bioactive agents, such bioactive agents can be selected as desired, including drugs, selection markers, detectable signals, other therapeutic agents, peptides, such as signal or cell targeting peptides, nucleic acid sequences, proteins (including antibodies), plasmids, viruses, viral vectors, small molecules, inorganic compounds, metals or derivatives thereof. Nucleic acids so linked can include antisense, siRNA, RNAi, aptamers or ribozymes.

Additionally, any inorganic or organic molecules, including amino acids, silicas, cytokines, such as interleukins, biologics and drugs may be added to the nucleic acid polymers to produce certain biological effects. Nucleic acids provide a variety of molecular attachment sites and therefore facilitate covalent, ionic and hydrogen bonding, as well as Van der Wals attachments, or other forms of attachment.

In addition, the nucleic acids may be methylated, ethylated, alkylated, or otherwise modified along the backbone to influence degradation rates. Generally, methylated, hemi-methylated, ethylated, or alkylated nucleic acids will degrade more slowly. Other backbone modifications affecting degradation rates include the use of heteroatomic oligonucleoside linkages as described in U.S. Pat. No. 5,677,437. Additionally, modifications may be used to prevent the nucleic acid from being transcribed or translated in a given tissue or organism. In addition, the nucleic acids may be capped to prevent degradation. Such caps are generally located at or near the termini of the nucleic acid chains. Examples of capping procedures are included in U.S. Pat. Nos. 5,245,022 and 5,567,810.

One aspect of the invention is directed to a matrix comprising nucleic acids that include X-shape, T-shape, Y-shape or dumbbell-shape, which nucleic acids can be used as building blocks for new, designer biomaterials. Thus the nucleic acid(s) have different shapes and one or more shapes can be utilized as a monomer (e.g., building block) for constructing DL-NAMs. In one embodiment, branched nucleic acids are all of one shape (X-, Y-, dumbbell- or T-shape), which nuclei acids are used as monomers. In some embodiments, branched nucleic acids are prepared through the hybridization of the complimentary sequences of the pre-designed oligonucleotides (Table 3). In some embodiments, the nucleic acids are DNA, RNA, PNA, LNA or TNA. In additional embodiments, one or more combinations of such nucleic acids can be utilized as building blocks. In further embodiments, the monomers are linked to other monomers by ligation. Therefore, the monomers can undergoe a ligation reaction faciliated by a nucleic acid ligase.

Furthermore, the nucleic acids are capable of undergoing enzymatic reactions. In some embodiments, the reactions include reactions by enzymes, wherein said one or more enzyme is a DNA polymerase, RNA reverse transcriptase, terminal transferase, DNA ligase, RNA ligase, exonuclease, ribonuclease, endonuclease, polynucleotide kinase, DNA methylase, or DNA ubiquitinase. Furthermore, reactions include any reaction wherein one or more enzyme is an enzyme that shortens nucleic acids, lengthens nucleic acids, amplifies nucleic acids, labels nucleic acids, or a combination of reactions/enzymes thereof.

X-Shape

In one aspect of the present invention, DL-NAMs are comprised entirely or at least in part of branched nucleic acids that are X-shape nucleic acids. In one embodiment, the X-shape nucleic acid is DNA. In yet another embodiment, the matrix is comprised of X-shape DNA and/or RNA, or analogs/derivative thereof. In another embodiment, the matrix is comprised of X-shape DNA, and linear DNA, RNA or PNA. In one preferred embodiment, the matrix is nearly entirely comprised of nucleic acids. In yet another embodiment, the X-shape nucleic acids are RNA.

In one embodiment, four different oligonucleotides with complimentary sequences, termed as X01, X02, X03, and X04 (Table 3), are hybridized with each other through an annealing process to achieve the final X-DNA. Furthermore, a plurality of said X-DNA can be linked via same or different linear DNA, which can be varied by sequence and/or size, to construct a unique DL-NMAs.

In certain aspects of the invention, the X-DNA terminal ends are designed with sticky ends that are capable of undergoing an enzymatic reaction. In one embodiment, the enzymatic reaction is a ligation reaction with a DNA ligase, which results in covalent linkage of two or more monomers. In yet a further embodiment, the DNA ligase is a T4 DNA ligase.

In one embodiment, X-DNA molecules can be designed and synthesized in such a way that each arm of the X-DNA possessed a complimentary sticky end whose sequences are palindromic.

X-shaped nucleic acid molecules can be synthesized by mixing equal amounts of four oligonucleotide strands. The nomenclature is as follows: X₀ₐ, X_{0b}, X_{0c}, and X_{0d} are the four corresponding single oligonucleotide chains that form a X₀-nucleic acid molecule (X₀). Similarly, X₁ₐ, X_{1b}, X_{1c}, and X_{1d} are the four corresponding single oligonucleotide chains that form an X₁-nucleic acid molecule (X₁); and Xₙₐ, X_{nb}, X_{nc}, and X_{nd} are the four corresponding single oligonucleotide chains that form a Xₙ-shaped nucleic acid molecule (Xₙ). The reactions can be the following: X₀ₐ+X_{0b}+X_{0c}+X_{0d}→X₀, X₁ₐ+X_{1b}+X_{1c}+X_{1d}→X₁, and Xₙₐ+X_{nb}+X_{nc}+X_{nd}→Xₙ, etc. (see FIGS. 24 and 25).

For the X-shaped nucleic acid molecule, the region 2 of each polynucleotide is complementary to region 3 of one of the other three polynucleotides. For example, with reference to the sequences in Tables 5 and 6: region 2 of SEQ ID NO: 16 is complementary to region 3 of SEQ ID NO: 19, region 2 of SEQ ID NO: 17 is complementary to region 3 of SEQ ID NO: 16, region 2 of SEQ ID NO: 18 is complementary to region 3 of SEQ ID NO: 17; and region 2 of SEQ ID NO: 19 is complementary to region 3 of SEQ ID NO: 18.

In one embodiment, the length of each of the regions can vary. For example, in some embodiments, the second and/or third regions for the X-shaped nucleic acid molecule and the second and/or fourth regions of the T-shaped nucleic acid molecules are about 13 nucleotides each in length. In some embodiments, the lengths of these regions may be 7, 8, 9, 10, 11, 12, 13, 14, 15, 16, 17, 18, 19, or 20 nucleotides in length. In some embodiments, these regions may be larger than 20 nucleotides in length, for example they may be about 25, 30, 35, 40, 45, or 50 nucleotides in length.

TABLE 1
Sequences of Oligonucleotides

TABLE 2A
Sequence Table

| SEQ ID | |
|---|---|
| NO | Sequence |
| 4 | 5′-ACTGCTGGATCGTATGCGTAGTCTGGACGTCTACCGTGT-3′ |
| 5 | 5′-CAGTGCAGGCTACGCATACCATCCAG-3′ |
| 6 | 5′-ACTGACACGGTAGACGTCCAGCCTGC-3′ |
| 7 | 5′-ACTG-3′ |
| 8 | 5′-CAGT-3′ |
| 9 | 5′-CTGGATCGTATGCGTA′3′ |
| 10 | 5′-GCAGGCT-3′ |
| 11 | 5′-ACACGGTAGACGTCCA-3′ |
| 12 | 5′-GTC-3′ |
| 13 | 5′-TGGACGTCTACCGTGT-3′ |
| 14 | 5′-ACGCATACCATCCAG-3′ |
| 15 | 5′-GCCTGC-3′ |

TABLE 2B
Sequences of Oligonucleotides

| | SEQ ID | | | |
|---|---|---|---|---|
| Strand | NO: | Region 1 | Region 2 | Region 3 |
| X₀ₐ | 16 | 3′-TCGA | AGGCTGATTCGGT | TAGTCCATGAGTC-5′ |
| X_{0b} | 17 | 3′-AATT | GACTCATGGACTA | TCATGCGGATCCA-5′ |
| X_{0c} | 18 | 3′-AGCT | TGGATCCGCATGA | CATTCGCCGTAAG-5′ |
| X_{0d} | 19 | 3′-GATC | CTTACGGCGAATG | ACCGAATCAGCCT-5′ |

TABLE 3
Sequence Table

| SEQ ID NO | Sequence |
|---|---|
| 16 | 3′-TCGAAGGCTGATTCGGTTAGTCCATGAGTC-5′ |
| 17 | 3′-AATTGACTCATGGACTATCATGCGGATCCA-5′ |
| 18 | 3′-AGCTTGGATCCGCATGACATTCGCCGTAAG-5′ |
| 19 | 3′-GATCCTTACGGCGAATGACCGAATCAGCCT-5′ |
| 20 | 3′-TCGA-5′ |
| 21 | 3′-AATT-5′ |
| 22 | 3′-AGCT-5′ |
| 23 | 3′-GATC-5′ |
| 24 | 3′-AGGCTGATTCGGT-5′ |
| 25 | 3′-GACTCATGGACTA-5′ |
| 26 | 3′-TGGATCCGCATGA-5′ |
| 27 | 3′-CTTACGGCGAATG-5′ |
| 28 | 3′-TAGTCCATGAGTC-5′ |
| 29 | 3′-TCATGCGGATCCA-5′ |
| 30 | 3′-CATTCGCCGTAAG-5′ |
| 31 | 3′-ACCGAATCAGCCT-5 |

Thus, X-DNA can ligate with each other via T4 DNA ligase, resulting in highly branched dendrimer structure. In some embodiments, linear nucleic acids, Y-shape, T-shape, dumbell-shape or dendrimer shape nucleic acids having the necessary sticky ends can also be incorporated into branched dendrimer structure (e.g., DL-NAMs) formed of X-shape nucleic acids. Therefore, in some embodiments, the matrix is comprised of X-shape and one or more other shapes in a ratio of each monomer that is preselected as desired.

### Y-Shape

In another aspect, DL-NAMs are comprised of Y-shape nucleic acids. In one embodiment, the Y-shape nucleic acid is DNA. In yet another embodiment, DL-NAMs Y-shape DNA and/or RNA, or analogs/derivatives thereof. In another embodiment, DL-NAMs are comprised of Y-shape DNA, and linear DNA or RNA. In one embodiment, DL-NAMs are comprised entirely of nucleic acids that are Y-shape. In a further embodiment, DL-NAMs are comprised of Y-shape and X-shape nucleic acids, in a ratio that is preselected as desired.

In one embodiment, DL-NAMs are assembled by ligation of Y-DNA molecules, whose sequences are specifically designed so that ligations between Yi and Y-DNA could only occur when i≠j, where i and j refer to the generation number n, for example, G1, G2, etc. The cohesive end of each oligonucleotide is non-palindromic, thus no self-ligations occurred. In addition, the ligation can only occur in one direction, that is, Y₁→Y₂→Y₃→Y₄ and so on. Furthermore, when Y₀ is ligated to Y₁ with a 1:3 molar stoichiometry, one Y₀ was linked with three Y₁, forming the first-generation DL-DNA. G1 is then ligated to six Y₂ (one Y₂ for each of the six free branches of G1), resulting in a second-generation DL-DNA (G2). The third (G3), fourth (G4), and higher generation DL-DNA were assembled in a similar way. Note that the assembled DL-DNA (Gn) had only one possible conformation due to the unidirectional ligation strategy. The general format of the n^{th}-generation DL-DNA is Gn=(Y₀)(3Y₁)(6Y₂) . . . (3×2ⁿ⁻¹Yₙ), where n is the generation number and Yn is the n^{th} Y-DNA. The total number of Y-DNA in an nth-generation DL-DNA is 3×2ⁿ⁻¹−2. The growth of DL-DNA from n^{th} generation to (n+1)^{th} generation requires a total of 3×2ⁿ new Yₙ₊₁-DNA.

Three specific polynucleotides are combined to form each Y-DNA. First, the free energy (deltaG) was calculated for a sequence. In general, a lower free energy is desired. However, intermediate-low deltaG are also considered. Second, the secondary, structure of the molecule is considered. In general, the least amount of secondary structure is desired. Third, it needs to be determined if the molecule would form a self-dimer, as it should 5 not form a self-dimer. Fourth, me length is considered, which can vary depending on the design goals. The molecule should be long enough to form stable DNA structure. For Y-DNA, it should be more than 8 nucleotides (nt) long. Fifth, the helix geometry should be considered. Half-turns should be considered as the quantum of DNA nanostructure. The length between two junctions should be 5*n bp, where n is 0, 1, 2, 3 etc. Next, the G/C content should be considered, hi one embodiment, sequences are chosen that constitute about 50% G/C. Last, the symmetry of the molecule should be considered. Sequence symmetry (e.g., as those occurring in Holliday junctions) of each arm should be avoided. For Y-DNA sequence design, all three oligonucleotides should be checked at the same time. In some embodiments, for X-DNA, complementary segements/arms are longer than 10, 11, 12, 13, 14, 15, 16, 17, 18, 19 or 20 nucleotides. A number of programs are available online which enable researchers to obtain detailed information regarding a DNA sequence, such as melting temperature, self-priming, secondary structure formation, calculations of free energy and alternate structure form a given sequence. Single stranded DNA folding software can also be used to check for complete complementarities of sequences and provide two-dimensional representations of the complex. Furthermore, input branched DNA can be modified to model after multiple strand folding by sealring the double-stranded open ends with a poly(NTP) hairpin or spacer. Examples of such sequence evaluation tools available online include: Fisher Scientific at woldwideweb.firsheroligos.com/oligo_calconyl.asp; IDTOligoAnalyzer at 207.32.43.70/biotools/oligocalc/oligocalc.asp; Mfold at bioinfo.rpi.edu/applications/mfold/; and WWTACGV2.38 at koubai.virus.kyotou.ac.jp/tacg2/tacg2.form.html.

One of the easiest and informative ways to characterize the sequences and the formation of dendrimer-like DNA is the conventional agarose gel electrophoresis. It is also possible to visualize the structure using high resolution microscopy techniques such as TEM or AFM, where the final structure is large enough to be resolved by such instruments.

In one example, 4 consecutive nucleotides were used as a unit in the checking process. For example, Target sequence: AGCTGAT

Check 1: AGCT (SEQ ID NO: 20). Since no other AGCT (SEQ ID NO: 20) sequence appears in that sequence, the first sequence symmetry check passes.

Check 2: GCTG. Since no other GCTG sequence appears in that sequence, the second sequence symmetry check passes.

Check 3: CTGA. Similarly, the third sequence symmetry check passes.

Check 4: TGAT. Similarly, the fourth sequence symmetry check passes.

Each polynucleotide may include three regions. A first region (region 1) of each polynucleotide may include nucleotides that will form a 5′ sticky end when a Y-DNA is formed. A “sticky end” is a single-stranded overhang portion of one of the polynucleotides. In various embodiments, the sticky ends for any Y-shape (as well as X-, T- or dumbbell-shape) nucleic acids can be 1, 2, 3, 4, 5, 6, 7, 8, 9, or 10 nucleotides. In some embodiments, a polynucleotide may not have this sticky end. In general, a shorter sticky end will allow for less selectivity in binding. For example, a polynucleotide lacking a sticky end would have little to no selectivity. The sticky end in some embodiments is a four nucleotide sticky end.

The sticky end in some embodiments is a four nucleotide sticky end. In some embodiments, the sticky end includes, or is, TGAC (SEQ ID NO: 47), GTCA (SEQ ID NO: 48), CGAT (SEQ ID NO: 49), ATCG (SEQ ID NO: 50), GCAT (SEQ ID NO: 51), ATGC (SEQ ID NO: 52), TTGC (SEQ ID NO: 53), GCAA (SEQ ID NO: 54), or GGAT (SEQ ID NO: 55) (e.g., Tables 4, 5 or 6).

The second region (region 2) of each polynucleotide is complementary to the third region (region 3) of one of the other two polynucleotides that form the Y-DNA. The third region of each polynucleotide is complementary to the second region of the other of the other two polynucleotides of Y-DNA. For example, with reference to the sequences in Tables 4A and 4B: region 2 of SEQ ID NOs 32-36, represented by SEQ ID NO: 36, is complementary to region 3 of SEQ ID NOs 32-36, represented by SEQ ID NO: 41, region 3 of SEQ ID NOs 32-36, represented by SEQ ID NO: 37, is complementary to region 2 of SEQ ID NOs 37-41, represented by SEQ ID NO: 38; and region 2 of SEQ ID NOs 32-36, represented by SEQ ID NO: 40, is complementary to region 3 of SEQ ID NOs 37-41, represented by SEQ ID NO 39.

The second region (region 2) of each polynucleotide is complementary to the third region (region 3) of one of the other two polynucleotides that form the Y-DNA. The third region of each polynucleotide is complementary to the second region of the other of the other two polynucleotides of Y-DNA. For example, with reference to the sequences in Tables 4A and 4B: region 2 of SEQ ID NOs 32-36, represented by SEQ ID NO: 36, is complementary to region 3 of SEQ ID NOs 32-36, represented by SEQ ID NO: 41, region 3 of SEQ ID NOs 32-36, represented by SEQ ID NO: 37, is complementary to region 2 of SEQ ID NOs 37-41, represented by SEQ ID NO: 38; and region 2 of SEQ ID NOs 32-36, represented by SEQ ID NO: 40, is complementary to region 3 of SEQ ID NOs 37-41, represented by SEQ ID NO: 39, In one embodiment of the invention, each polynucleotide is 30 nucleotides in length, with the first region having 4 nucleotides, the second region having 13 nucleotides, and the third region also having 13 nucleotides. In some embodiments of the invention, the Y-shape polynucleotides include, essentially include, or are comprised of SEQ ID NOs: 22-SEQ ID NO: 62, or SEQ ID NOs: 63-72. With respect to any of the nucleic acid building blocks described herein (e.g., X-, Y-, T-, dumbbell-, dendrimer-shape), in various embodiments, the 5′ end can comprise a phosphorylation modification so as to include various labels disclosed herein (See, e.g., Alex488, BO630 probes/labels, supra).

In one embodiment of the invention, each polynucleotide is 30 nucleotides in length, with the first region having 4 nucleotides, the second region having 13 nucleotides, and the third region also having 13 nucleotides. In some embodiments of the invention, the Y-shape polynucleotides include, essentially include, or are comprised of SEQ ID NOs: 32-SEQ ID NO: 72, or SEQ ID NOs: 73-82. With respect to any of the nucleic acid building blocks described herein (e.g., X-, Y-, T-, dumbbell-, dendrimer-shape), in various embodiments, the 5′ end can comprise a phosphorylation modification so as to include various labels disclosed herein (See, e.g., Alex488, BO630 probes/labels, supra).

TABLE 4
Sequences of Oligonucleotides

| Strand | SEQ ID NO: | Region 1 | Region 2 | Region 3 |
|---|---|---|---|---|
| Y0a | 22 32 | 5′-TGAC | TGGATCCGCATGA | CATTCGCCGTAAG-3′ |
| Y1a | 23 33 | 5′-GTCA | TGGATCCGCATGA | CATTCGCCGTAAG-3′ |
| Y2a | 24 34 | 5′-ATCG | TGGATCCGCATGA | CATTCGCCGTAAG-3′ |
| Y3a | 25 35 | 5′-ATGC | TGGATCCGCATGA | CATTCGCCGTAAG-3′ |
| Y4a | 26 36 | 5′-GCAA | TGGATCCGCATGA | CATTCGCCGTAAG-3′ |
| Y0b | 27 37 | 5′-TGAC | CTTACGGCGAATG | ACCGAATCAGCCT-3′ |
| Y1b | 28 38 | 5′-CGAT | CTTACGGCGAATG | ACCGAATCAGCCT-3′ |
| Y2b | 29 39 | 5′-GCAT | CTTACGGCGAATG | ACCGAATCAGCCT-3′ |
| Y3b | 30 40 | 5′-TTGC | CTTACGGCGAATG | ACCGAATCAGCCT-3′ |
| Y4b | 31 41 | 5′-GGAT | CTTACGGCGAATG | ACCGAATCAGCCT-3′ |
| Y0c | 32 42 | 5′-TGAC | AGGCTGATTCGGT | TCATGCGGATCCA-3′ |
| Ylc | 33 43 | 5′-CGAT | AGGCTGATTCGGT | TCATGCGGATCCA-3′ |
| Y2c | 34 44 | 5′-GCAT | AGGCTGATTCGGT | TCATGCGGATCCA-3′ |
| Y3c | 35 45 | 5′-TTGC | AGGCTGATTCGGT | TCATGCGGATCCA-3′ |
| Y4c | 36 46 | 5′-GGAT | AGGCTGATTCGGT | TCATGCGGATCCA-3′ |

Sequence Table

| SEQ ID NO | Sequence |
|---|---|
| 32 | 5′-TGACTGGATCCGCATGACATTCGCCGTAAG-3′ |
| 33 | 5′-GTCATGGATCCGCATGACATTCGCCGTAAG-3′ |
| 34 | 5′-ATCGTGGATCCGCATGACATTCGCCGTAAG-3′ |
| 35 | 5′-ATGCTGGATCCGCATGACATTCGCCGTAAG-3′ |
| 36 | 5′-GCAATGGATCCGCATGACATTCGCCGTAAG-3′ |
| 37 | 5′-TGACCTTACGGCGAATGACCGAATCAGCCT-3′ |
| 38 | 5′-CGATCTTACGGCGAATGACCGAATCAGCCT-3′ |
| 39 | 5′-GCATCTTACGGCGAATGACCGAATCAGCCT-3′ |
| 40 | 5′-TTGCCTTACGGCGAATGACCGAATCAGCCT-3′ |
| 41 | 5′-GGATCTTACGGCGAATGACCGAATCAGCCT-3′ |
| 42 | 5′-TGACAGGCTGATTCGGTTCATGCGGATCCA-3′ |
| 43 | 5′-CGATAGGCTGATTCGGTTCATGCGGATCCA-3′ |
| 44 | 5′-GCATAGGCTGATTCGGTTCATGCGGATCCA-3′ |
| 45 | 5′-TTGCAGGCTGATTCGGTTCATGCGGATCCA-3′ |
| 46 | 5′-GGATAGGCTGATTCGGTTCATGCGGATCCA-3′ |
| 47 | 5′-TGAC-3′ |
| 48 | 5′-GTCA-3′ |
| 49 | 5′-CGAT-3′ |
| 50 | 5′-ATCG-3′ |
| 51 | 5′-GCAT-3′ |
| 52 | 5′-ATGC-3′ |
| 53 | 5′-TTGC-3′ |
| 54 | 5′-GCAA-3′ |
| 55 | 5′-GGAT-3′ |
| 56 | 5′-TGGATCCGCATGA-3′ |
| 57 | 5′-CATTCGCCGTAAG-3′ |
| 58 | 5′-CTTACGGCGAATG-3′ |
| 59 | 5′-ACCGAATCAGCCT-3′ |
| 60 | 5′-AGGCTGATTCGGT-3′ |
| 61 | 5′-TCATGCGGATCCA-3′ |
| 62 | TTGCTGGATCCGCATGACATTCGCCGTAAG-3′ |
| 63 | CGTTTGGATCCGCATGACATTCGCCGTAAG-3′ |
| 64 | ATGCTGGATCCGCATGACATTCGCCGTAAG-3′ |
| 65 | TGGATCCGCATGACATTCGCCGTAAG-3′ |
| 66 | GCATCTTACGGCGAATGACCGAATCAGCCT-3′ |
| 67 | GCAACTTACGGCGAATGACCGAATCAGCCT-3′ |
| 68 | CTTACGGCGAATGACCGAATCAGCCT-3′ |
| 69 | GCATAGGCTGATTCGGTTCATGCGGATCCA-3′ |
| 70 | TTGCAGGCTGATTCGGTTCATGCGGATCCA-3′ |
| 71 | AACGAGGCTGATTCGGTTCATGCGGATCCA-3′ |
| 72 | AGGCTGATTCGGTTCATGCGGATCCA-3′ |

In one embodiment of the invention, each polynucleotide is 30 nucleotides in length, with the first region having 4 nucleotides, the second region having 13 nucleotides, and the third region also having 13 nucleotides. In some embodiments of the invention, the Y-shape polynucleotides include, essentially include, or are comprised of SEQ ID NOs: 32-SEQ ID NO: 72, or SEQ ID NOs: 73-82. With respect to any of the nucleic acid building blocks described herein (e.g., X-, Y-, T-, dumbbell-, dendrimer-shape), in various embodiments, the 5′ end can comprise a phosphorylation modification so as to include various labels disclosed herein (See, e.g., Alex488, BO630 probes/labels, supra).

In certain aspects of the invention, the Y-DNA terminal ends are designed with sticky ends as described above that are capable of undergoing an enzymatic reaction. In one embodiment, the enzymatic reaction is a ligation reaction with a DNA ligase. In yet a further embodiment, the DNA ligase is a T4 DNA ligase.

In one embodiment, Y-shape nucleic acid building blocks are joined end-to-end to produce a dumbell shaped building block or dendrimer like nucleic acid e.g, FIGS. 9 and 30B)

### T-Shape

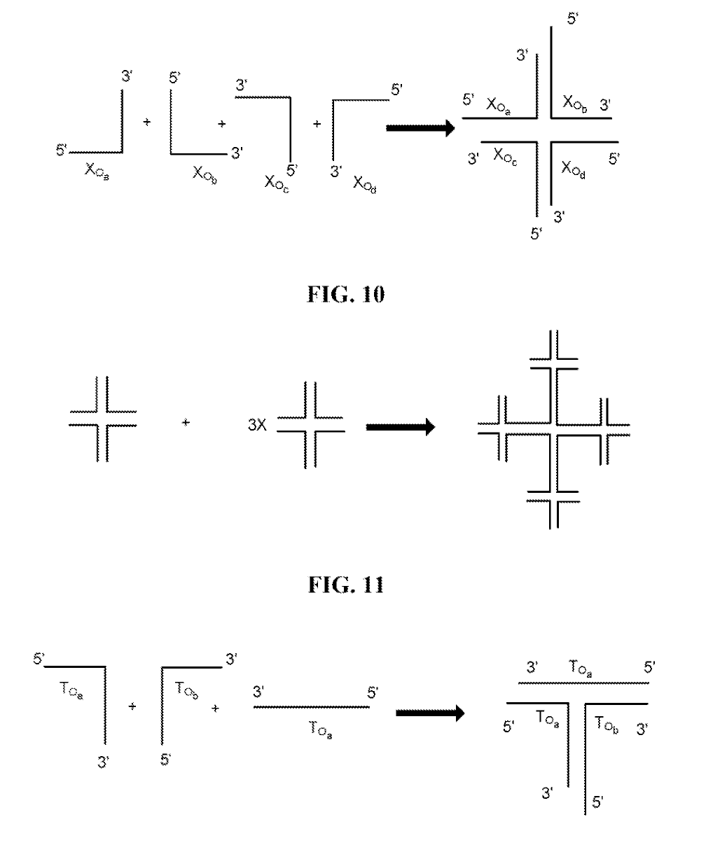

In yet another aspect, the nucleic acids forming a matrix are T-shape nucleic acids (FIG. 10). In one embodiment, the T-shape nucleic acids are DNA. In yet another embodiment, the matrix comprises T-shape DNA and/or RNA, or analogs/derivatives thereof. In addition, a matrix can be comprised of T-shape and one or more different shapes of nucleic acids, including X-, Y-, dumbell- or dendrimer-shape nucleic acids, as well as a combination thereof.

In one embodiment, the T-shape nucleic acids have a tensile strength selected from 50, 51, 52, 53, 54, 55, 56, 57, 58, 59, 60, 61, 62, 63, 64, or 65%. In addition, the T-shape nucleic acids can have a degree of swelling selected from 100, 105, 110, 115, 120, 125, 130, or 135%. For the T-shaped nucleic acid molecule, the second region (region 2) of each polynucleotide is complementary to the fourth region (region 4) of one of the other two polynucleotides. The fourth region of each polynucleotide is complementary to the second region of the other of the other two polynucleotides of T-shaped nucleic acid molecule. The third region is either absent or is a linker to permit formation of the T-shaped configuration. For example, with reference to the sequences in Tables 4 and 5A: region 2 of SEQ ID NO: 36 is complementary to region 4 of SEQ ID NO: 34, region 4 of SEQ ID NO: 36 is complementary to region 2 of SEQ ID NO: 35, and region 2 of SEQ ID NO: 34 is complementary to region 4 of SEQ ID NO: 35.

T-shaped nucleic acid molecules can be synthesized by mixing equal amounts of three oligonucleotide strands. The nomenclature is as follows: T₀ₐ, T_{0b}, and T_{0c} are the three corresponding single oligonucleotide chains that form a T₀-nucleic acid molecule (T₀). Similarly, T₁ₐ, T_{1b}, and T_{1c} are the three corresponding single oligonucleotide chains that form a T₁-nucleic acid molecule (T₁); and Tₙₐ, T_{nb}, and T_{nc} are the three corresponding single oligonucleotide chains that form a Tₙ-shaped nucleic acid molecule (Tₙ). The reactions can be the following: T₀ₐ+T_{0b}+T_{0c}→T₀, T₁ₐ+T_{1b}+T_{1c}→T₁, and Tₙₐ+T_{nb}+T_{nc}→Tₙ, etc. (see FIGS. 10 and 26).

In one embodiment, the T-shape nucleic acids have a tensile strength selected from 50, 51, 52, 53, 54, 55, 56, 57, 58, 59, 60, 61, 62, 63, 64, or 65%. In addition, the T-shape nucleic acids can have a degree of swelling selected from 100, 105, 110, 115, 120, 125, 130, or 135%. For the T-shaped nucleic acid molecule, the second region (region 2) of each polynucleotide is complementary to the fourth region (region 4) of one of the other two polynucleotides. The fourth region of each polynucleotide is complementary to the second region of the other of the other two polynucleotides of T-shaped nucleic acid molecule. The third region is either absent or is a linker to permit formation of the T-shaped configuration. For example, with reference to the sequences in Tables 4 and 5A: region 2 of SEQ ID NO: 46 is complementary to region 4 of SEQ ID NO: 44, region 4 of SEQ ID NO: 46 is complementary to region 2 of SEQ ID NO: 45, and region 2 of SEQ ID NO: 44 is complementary to region 4 of SEQ ID NO: 45

In yet other embodiments, gels can be comprised of one or more differently shaped nucleic acids, including X-, Y-, T-, dumbell- or dendrimer-shaped DNA (e.g., Y- and X-DNA, or Y- and T-DNA or X- and T-DNA). In yet further embodiments, applicable to any matrix disclosed herein, gels can be comprised of nucleic acids that include DNA, RNA, PNA, TNA, or a combination thereof.

TABLE 5
Example of Oligonucleotides used to construct X-, Y-
and T-nucleic acid building blocks.

| | | | SEQ. |
|---|---|---|---|
| Strand | Segment 1 | Segment 2 | ID. NO. |
| X01 | 5′-p-ACGT | CGA CCG ATG AAT AGC GGT CAG ATC CGT ACC TAC TCG-3′ | 73 |
| X02 | 5′-p-ACGT | CGA GTA GGT ACG GAT CTG CGT ATT GCG AAC GAC TCG-3′ | 74 |
| X03 | 5′-p-ACGT | CGA GTC GTT CGC AAT ACG GCT GTA CGT ATG GTC TCG-3′ | 75 |
| X04 | 5′-p-ACGT | CGA GAC CAT ACG TAC AGC ACC GCT ATT CAT CGG TCG-3′ | 76 |
| Ya | 5′-p-ACGT | CGA CCG ATG AAT AGC GGT CAG ATC CGT ACC TAC TCG-3′ | 77 |
| Yb | 5′-p-ACGT | CGA GTC GTT CGC AAT ACG ACC GCT ATT CAT CGG TCG-3′ | 78 |
| Yc | 5′-p-ACGT | CGA GTA GGT ACG GAT CTG CGT ATT GCG AAC GAC TCG-3′ | 79 |
| Ta | 5′-p-ACGT | CGA CAG CTG ACT AGA GTC ACG ACC TGT ACC TAC TCG-3′ | 80 |
| Tb | 5′-p-ACGT | CGA GTC GTT CTC AAG ACG TAG CTA GGA CTC TAG TCA GCT GTC G-3′ | 81 |
| Tc | 5′-p-ACGT | CGA GTA GGT ACA GGT CGT CGT CTT GAG AAC GAC TCG-3 | 82 |

Note that p represents the phosphorylation on the 5′ end of the oligonucleotide.

To confirm the formation of these branched DNA building blocks, a gel electrophoretic migration-shift assay (GEMSA) coupled with a DNA-specific fluorescent dye (SYBR I) was employed. In general, lower salt concentrations can be used for more specific base-pairing, while higher salt concentrations favor strong electro-static intereactions. Ionic influences on DNA are familiar to one of ordinary skill in the art. (See, e.g., Macromolecules, 1997, 30: 5763; J. Phys. Chem. 2006; 110: 2918-2926; Biophys. J. 1996; 70: 2838-46.

Similar experiments as above were also performed with X- Y- and T-DNA, which led to controlled-assembled of three dimensional structures. For ligation, manufacturer's protocols were followed. Mg++ was added for ATP. Hydrogel gelation correlated with ligase activity. For example, by using twice the amount of ligase (e.g., 60 Units), the DNA hydrogel was completely formed within 30 minutes. A typical example of ligase reaction utilized Ligase 10× buffer which has a composition of 300 mM Tris-HCl (pH 7.8), 100 mM Mg Cl2, 100 mM DTT and 10 mM ATP. T4 DNA ligase is supplied with 10 mM Tris-HCl (pH 7.4), 50 mM KCl, 1 mM DTT, 0.1 mM EDTA and 50% glycerol.

Dendrimer Structures

As almost all nucleic acid molecules are either linear or circular, to rationally construct nucleic acid biomaterials, additional shapes of nucleic acids as basic building blocks must be first constructed. In addition, these nucleic acid building blocks must be readily incorporated into larger structures in a controlled manner. Thus, in one aspect of the invention, dendrimer like nucleic acid structures are assembled to provide a biomaterial compound.

In other aspects of the invention branched or DL-NAMs are utilized to form dendrimer structures. Synthesizing monodisperse polymers demands a high level of synthetic control which is achieved through stepwise reactions, building the dendrimer up one monomer layer, or “generation,” at a time. Each dendrimer consists of a multifunctional core molecule with a dendritic wedge attached to each functional site. The core molecule is referred to as “generation 0.” Each successive repeat unit along all branches forms the next generation, “generation 1,” “generation 2,” and so on until the terminating generation (e.g., FIG. 4A and FIG. 7). Such a level of control is achieved through controlled assembly through the sticky ends of the multimer building blocks utilized to assemble a DL-NAM.

There are two defined methods of dendrimer synthesis, divergent and convergent. In the divergent method the molecule is assembled from the core to the periphery; while in the convergent method, the dendrimer is synthesized beginning from the outside and terminating at the core. In either method the synthesis requires a stepwise process, attaching one generation to the last, purifying, and then changing functional groups for the next stage of reaction. For example, in FIG. 7, the shaded inner core represents one step, followed by the unshaded “Y” molecules as an additional and subsequent step, and finally the stipeled “Y” molecules as a further additional and subsequent step. This functional group transformation is necessary to prevent unbridled polymerization. Such polymerization can lead to a highly branched molecule which is not monodisperse—otherwise known as a hyperbranched polymer.

In the divergent method, the surface groups initially are unreactive or protected species which are converted to reactive species for the next stage of the reaction. In the convergent approach the opposite holds, as the reactive species must be on the focal point of the dendritic wedge.

Due to steric effects, continuing to react dendrimer repeat units leads to a sphere shaped or globular molecule until steric overcrowding prevents complete reaction at a specific generation and destroys the molecule's monodispersity. The number of possible generations can be increased by using longer spacing units in the branches of the core molecule. The monodispersity and spherical steric expansion of dendrimers leads to a variety of interesting properties. The steric limitation of dendritic wedge length leads to small molecular sizes, but the density of the globular shape leads to fairly high molecular weights. The spherical shape also provides an interesting study in molecular topology. Dendrimers have two major chemical environments, the surface chemistry due to the functional groups on the termination generation, which is the surface of the dendritic sphere, and the sphere's interior which is largely shielded from exterior environments due to the spherical shape of the dendrimer structure. The existence of two distinct chemical environments in such a molecule implies many possibilities for dendrimer applications.

As such, hydrophobic/hydrophilic and polar/nonpolar interactions can be varied in the two environments. The existence of voids in the dendrimer interior furthers the possibilities of these two heterogeneous environments playing an important role in dendrimer chemistry. Therefore, in a further embodiment dendrimer structures can accept molecules in the void spaces in addition to or alternative to the linkage to one or more arm portions of one or more terminal monomer (e.g., Y-shape) nucleic acid molecules. Non-nucleic acid dendrimers have found actual and potential use as molecular weight and size standards, gene transfection agents, as hosts for the transport of biologically important guests, and as anti-cancer agents, to name but a few. Much of the interest in dendrimers involves their use as catalytic agents, utilizing their high surface functionality and ease of recovery. Dendrimers' globular shape and molecular topology, however, make them highly useful to biological systems. Utilizing nucleic acid molecules as building blocks for dendrimer construction and further linked to biologically active agents provides wholly new opportunities in biotechnology and medicine.

In some aspects of the invention the dendrimer structures can be formed at any stage during a step-wise process of formulation to provide a multivalent structure. Such a dendrimer structure can be composed of multimers that are Y-, X-, T-, or dumbbell shape. In one embodiment, the dendrimer is formed of entirely one shape. In other embodiments, the dedrimer is formed of one or more different shape multimers.

In one embodiment, the multimers forming said dendrimer structure are DNA multimers. In another embodiment, the dendrimer structure is comprised of DNA and/or RNA. As indicated above, dendrimers are formed through step-wise addition of different nucleic acid monomers (i.e., building blocks), where for example, nucleic acid monomer ends provide overhangs for subsequent ligation reactions thus expanding the three-dimensional structure of the expanding dendrimer structure.

Therefore, in selection various nucleic acids, monomers of different length can be utilized to form dendrimers having a different internal and surface area network. Furthermore, the various sticky ends and/or monomer units can provide a substrate for linking to one or a plurality of biologically active agents. Such biologically active agents are known in the art or described herein.

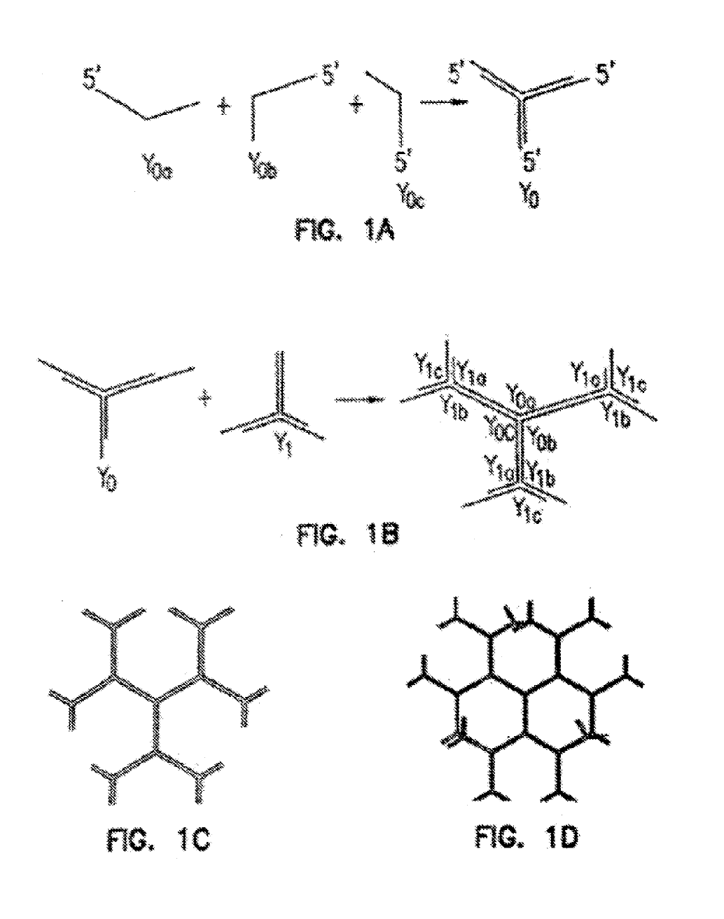

In one aspect of the invention, a method is directed to controlled assembly of dendrimer-like DNA (DL-DNA) from Y-shaped DNA (Y-DNA) FIGS. 1A-D. In one embodiment, the resulting DL-DNA is stable and monodisperse. In a further embodiment, the DL-NAMs are isotropic or anisotropic, thus capable of linkage to other compounds FIG. 7. In some embodiments, multimers are joined together to form a honeycomb structure FIG. 1D.

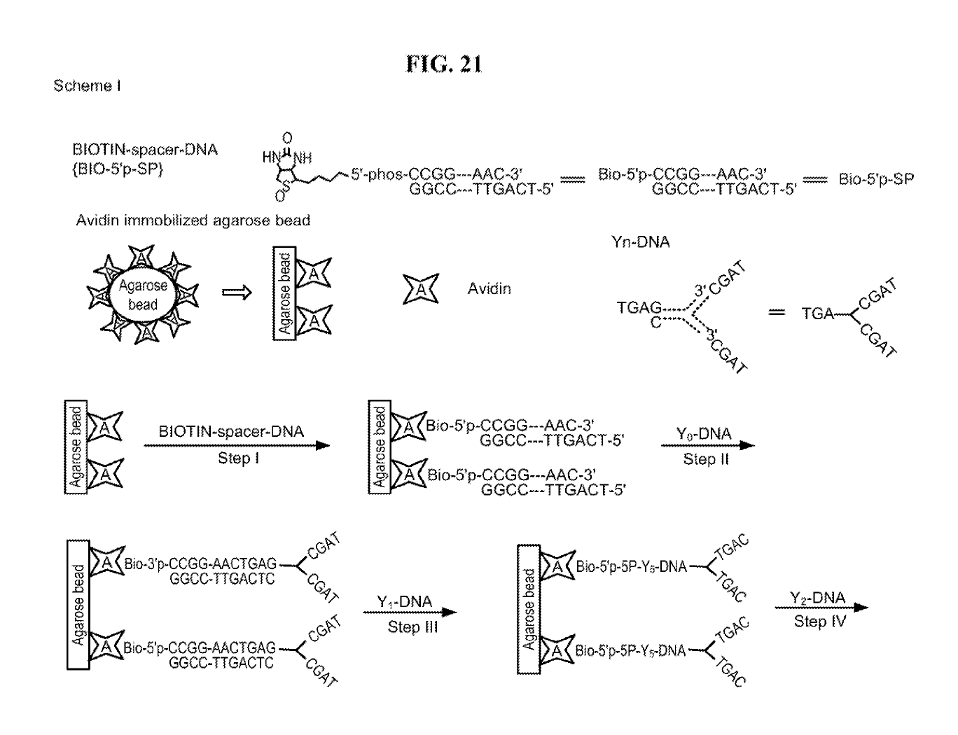

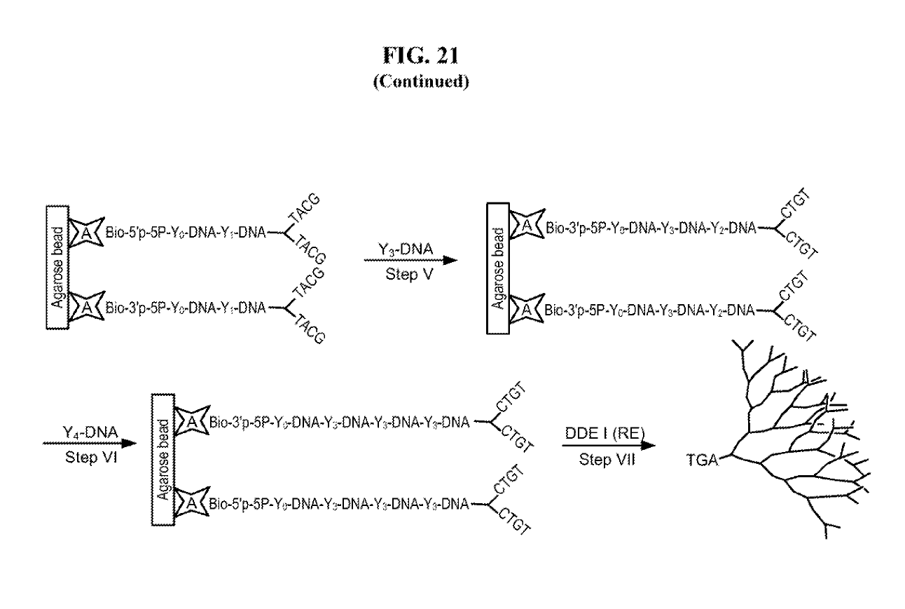

In some embodiments, DL-NAMs comprise branched and linear nucleic acids, as described herein above. For example, a linear spacer DNA can be linked to a branched building block DNA (e.g., X-, Y-, T-, dumbbell-shape), whereby such linkage provides G₀ in dendrimer construction (e.g., FIG. 21 and Example 6). A spacer molecule can be DNA, RNA, PNA or peptide. Furthermore, in one example FIG. 21, a spacer molecule can be linked to a member of a binding-pair such as avidin/biotin, where the cognate member is linked to a solid support (e.g., agarose bead).

While avidin/streptavidin to biotin is utilized for illustrative purposes, it should be understood that binding-pairs known in the art can be utilized in such a solid phase assembly process.

Binding partners are generally components capable of specific binding. The binding partner may be a protein, which may be an antibody or an antigen. The binding partner may be a member of a specific binding pair (“sbp member”), which is one of two different molecules, having an area on the surface or in a cavity which specifically binds to and is thereby defined as complementary with a particular spatial and polar organization of the other molecule. The members of the specific binding pair will usually be members of an immunological pair such as antigen-antibody, although other specific binding pairs such as biotin-avidin, hormones-hormone receptors, enzyme-substrate, nucleic acid duplexes, IgG-protein A, polynucleotide pairs such as DNA-DNA, DNA-RNA, and are included within the scope of sbp member.

Accordingly, specific binding involves the specific recognition of one of two different molecules for the other compared to substantially less recognition of other molecules. Such Sbp can be utilized in solid phase assembly of DL-NAMs as illustrated in FIG. 21. Sbp members are known in the art, such as disclosed in U.S. Pat. No. 7,122,384; 6,589,798; 6,586,193; 6,511,809; or 6,214,560.

In one embodiment, such solid supports are arranged in an array format in a process of producing DL-NAMs in a highthroughput format. For example, wells of microtiter plates can be coated with a member of a binding pair, with a spacer molecule comprising the cognate member and said microtiter plates can be processed in an automated system, where computer executable logic provides instructions for addition of solutions/reagents necessary for DL-NAM synthesis in a stepwise fashion. In some embodiments 1, 5, 10, 20, 50, 100 or 500 microtiter plates can be processed. Furthermore, microtiter formats include plates have more than 90, more than 180, more than 360 or more than 720 wells/plate.

Multivalency

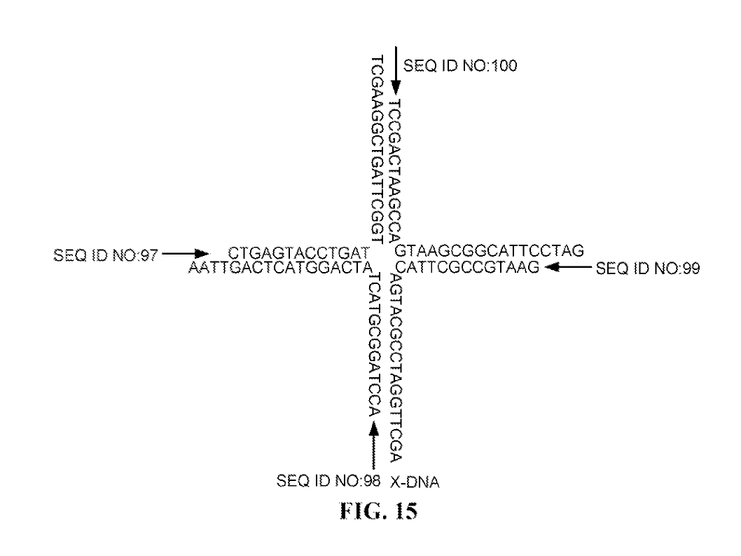

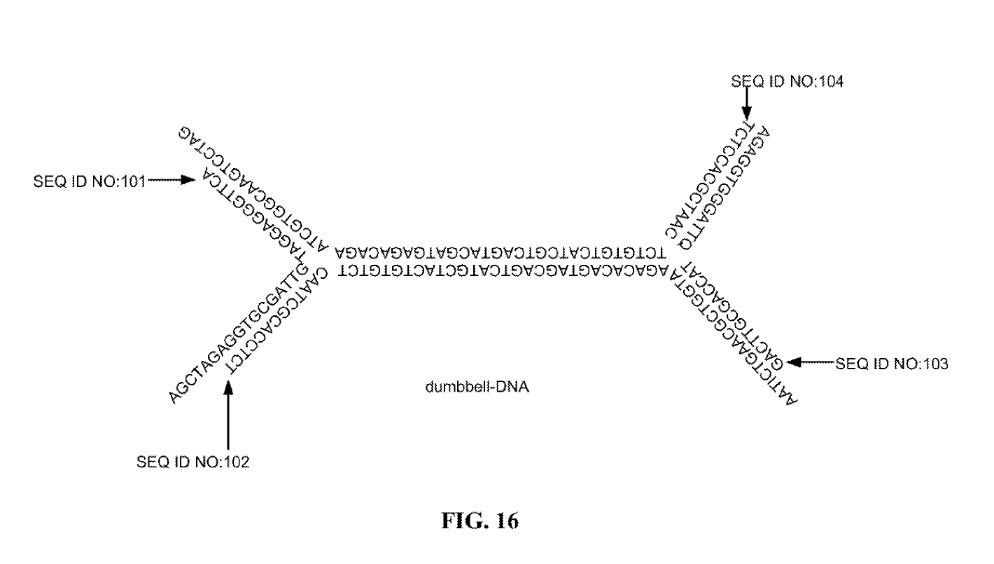

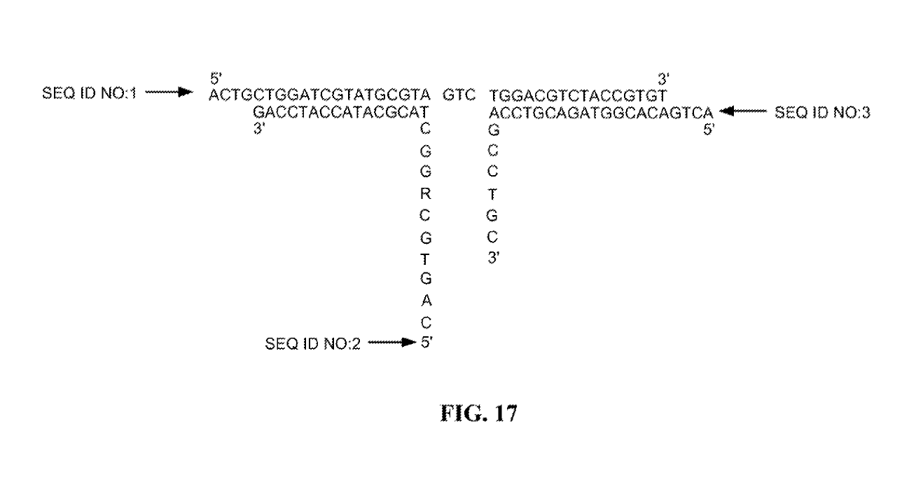

Branched nucleic acids described herein are dendrimer like and thus by combining such nucleic acids in a step-wise or all in one fashion, dendrimer structure are formed (i.e., DL-NAMs). Furthermore, in some embodiments, the various arms of said Y-shape nucleic acids, as comprised on in a dendrimer structure, are linked to one or more biologically active agents, which agents are described herein. Thus, in one embodiment, the arms are linked to a targeting peptide or signal peptide, a selection marker, a detectable label, a small compound, a drug, a pharmaceutical or to a plasmid or viral vector, or virus. It should be apparent to one of skill in the art that the Y-shape nucleic acids forming said dendrimer (DL-NAMs) afford attachment of multiple same or different compounds (FIGS. 14-16). In other words, the dendrimer structures are anisotropic and/or multivalent. As multivalent structures, DL-NAMs provide a means for targeting specific cell/tissue, utilizing a nanostructure that is water-soluble and biocompatible, thus providing an efficient means for cellular targeting, sufficient cellular uptake and delivery of one or more bioactive agents.

For example, the present dendrimer/based “nanodevices” (i.e., DL-NAMs) that contain the necessary basic components, such as a targeting agent that would specifically bind to a tumor, a fluorescent molecule for tracking its presence in cells, and a drug to kill the targeted cells, can be utilized to reduce toxicity associated with radiation and chemotherapy, attendant to most cancer treatment regimes. DL-NAMs of the invention, by virtue of their modularity and controlled synthesis in numbered “generations” can be custom tailored for a particular disease or treatment. DL-NAMs are a highly suitable drug carriers because of their biocompatible properties and their nanometer size, dimension, and structural architecture, which mimic certain biomolecules.

Therefore, in some embodiments, DL-NAMs carry an anticancer drug and a targeting moiety to a cancer cell, whereby targeted drug delivery is founded on the principle that if a receptor is expressed specifically or in excess on the surface of a cancer cell, the dendrimer carrying a drug and a ligand for the receptor travels through the circulation, binds specifically to the cancerous cells, and delivers the drug to induce programmed cell death. Furthermore, owing to the multivalent character of the DL-NAMs of the invention conjugation of several molecules of a targeting agent onto the dendrimer will result in an increase in the dendrimer's avidity for binding to the targeted cells through multivalent interaction because of the binding of multiple targeting molecules to their receptor. While not limited to any particular cancer-expressed marker, in some embodiments, DL-NAMs comprise a targeting moiety that is Folic acid receptor (FAR), which is overexpressed on the surface of a variety of malignancies, such as cancer of the head and neck and of the ovary.

In one embodiment the Y-shape, X-shape, T-shape or dumbbell-shape arms are attached to a peptide moiety comprising an adenovirus core peptide, a synthetic peptide, an influenza virus HA2 peptide, a simian immunodeficiency virus gp32 peptide, an SV40 T-Ag peptide, a VP22 peptide, a Tat peptide, a Rev peptide, DNA condensing peptide, DNA protection peptide, endosomal targeting peptide, membrane fusion peptide, nuclear localization signaling peptide, a protein transduction domain peptide or any combination thereof.

Such peptides can be selected based on their properties related including but not limited to properties to overcome various cellular barriers, peptides from viruses chosen from disparate viruses to preclude capsid assembly, providing an extra amino acid Cys at C-terminal end to facilitate conjugation or pre-determining ratios of peptide to DL-NAM to provide DL-NAMs with specific peptides linked thereonto.

Nonlimiting examples of peptides include, SV40 NLS peptide or semian virus 40 large tumor antigen (FKKKRKVEDPYC; SEQ ID NO: 83), which is a nuclear localization peptide (NLS) that can translocate other molecules from cytosol to the nucleus through the nuclear membrane.

In some embodiments the delivery peptide is HIV Tat. The trans-activating transcriptional activator (Tat) which is an 86 amino acid protein from HIV-1. The effective part of Tat for translocation can be as short as 13 amino acids (TAT48-60: GRKKRRQRRRPPQ; SEQ ID NO: 84). Tat can offer efficient intracellular delivery of both macromolecules and small particles. HIV Tat is ideal to overcome the plasma uptake barrier.

In some embodiments the delivery peptide is Adno mu peptide. Adenovirar core peptide mu (MRRAHHRRRRASHRRMRGG; SEQ ID NO: 85) functions as a nucleic acid condensing peptide and can be used to condense DNA for gene delivery due to its highly cationic properties. An important aspect of the invention is not a particular targeting peptide, but the multivalency of DL-NAMs. For example, an artisan will realize that receptors represent a simple alternative to the use of antibodies as targeting ligands for cell specific gene delivery, although antibodies are readily adaptable to DL-NAMs of the Mention. Additional cellular targeting peptides are known to one of skill in the art, such as disclosed in U.S. Pat. Nos. 6,649,407; 6,576,456; 6,548,634; See also, Aronsohn A I and Hughes J A. Nuclear localization signal peptides enhance cationic liposome-mediated gene therapy. J Drug Target. 1998; 5:163-169.

In some embodiments, DNA condensing peptides can be linked to DL-NAMs. For example, with advancement of genetic engineering and protein chemistry, many peptides can be designed de novo and synthesized accordingly. The condensing peptide (Y-WKC) has been successfully utilized for DNA delivery as a synthetic, DNA condensation peptide.

In another embodiment, the Y-shape, T-shape, X-shape or dumbbell shape nucleic acids are linked to one or more biologically active agents, including the preceding peptides, one or more selection markers, one or more detectable labels, one or more drugs, small compounds, or nucleic acid sequences or one or more copolymer compounds.

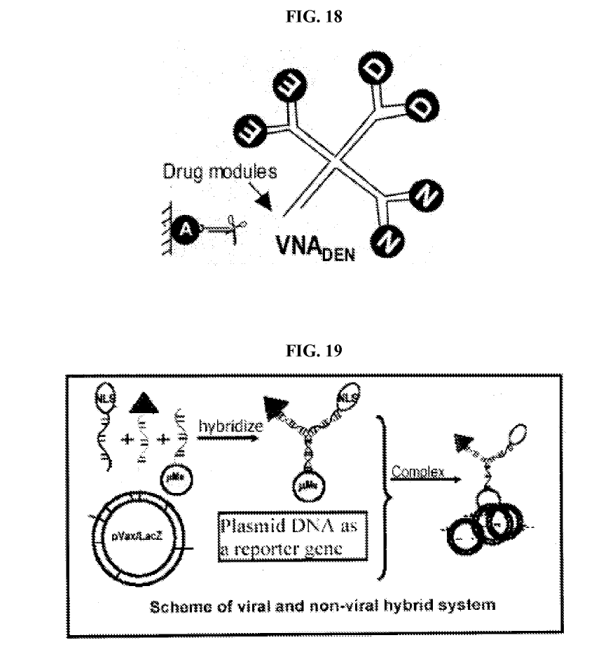

In certain embodiments, the dendrimer structures are linked or cross-linked to additional compounds selected from a group consisting of an adenovirus core peptide, a synthetic peptide, an influenza virus HA2 peptide, a simian immunodeficiency virus gp32 peptide, an SV40 T-Ag peptide, a VP22 peptide, a Tat peptide, and a Rev peptide. Such additional compounds are selected from a group consisting of DNA condensing peptide, DNA protection peptide, endosomal targeting peptide, membrane fusion peptide, nuclear localization signaling peptide, a protein transduction domain peptide or a combination thereof (FIGS. 18-19).

In one embodiment, the dendrimer structures are utilized in a method of delivering a biologically active agent to a cell, or to a subject. In another embodiment, the dendrimer structure comprises a linkage to a signal or targeting peptide as described herein above, as well as a bioactive agent having therapeutic properties (e.g., drug, siRNA, nucleic acid encoding a therapeutic protein). Additional targeting and delivery moieties are known in the art, such as disclosed in U.S. Pat. No. 7,122,525; 7,122,172, 7,097,856, 699176, 6992169, 6977075 or 6939528.

Labels and Selection Markers

In yet another embodiment, the dendrimer comprises a targeting peptide, a biologically active agent, a selection marker and a detectable label. Selection markers include antibiotics which are known in the art for both eukaryotic and prokaryotic cells, or disclosed herein. Infra. Therefore, as noted above, a dendrimer can provide a multivalent structure comprised of several distinct molecules that are bound to one or more arms of a one or more multimer nucleic acid molecules of which a dendrimer is composed. (e.g., FIGS. 18-20).

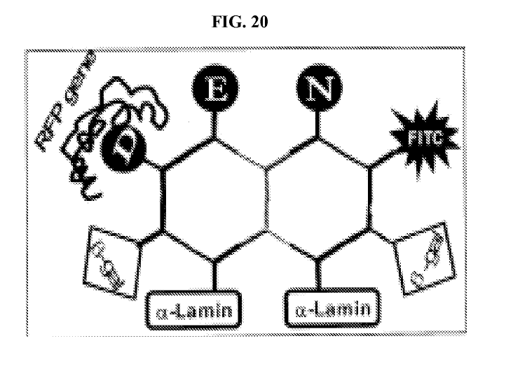

Specific examples of detectable molecules include radioactive isotopes such as p³² or H³, fluorophores such as fluorescein isothiocyanate (FITC) FIG. 20, TRITC, rhodamine, tetramethylrhodamine, R-phycoerythrin, Cy-3, Cy-5, Cy-7, Texas Red, Phar-Red, allophycocyanin (APC), epitope tags such as the FLAG or HA epitope, and enzyme tags such as alkaline phosphatase, horseradish peroxidase, I²-galactosidase, and hapten conjugates such as digoxigenin or dinitrophenyl, etc. Other detectable markers include chemiluminescent and chromogenic molecules, optical or electron density markers, etc. The probes can also be labeled with semiconductor nanocrystals such as quantum dots (i.e., Qdots), described in U.S. Pat. No. 6,207,392. Qdots are commercially available from Quantum Dot Corporation.

Additional examples of reagents which are useful for detection include, but are not limited to, radiolabeled probes, fluorophore-labeled probes, quantum dot-labeled probes, chromophore-labeled probes, enzyme-labeled probes, affinity ligand-labeled probes, electromagnetic spin labeled probes, heavy atom labeled probes, probes labeled with nanoparticle light scattering labels or other nanoparticles or spherical shells, and probes labeled with any other signal generating label known to those of skill in the art. Non-limiting examples of label moieties useful for detection in the invention include, without limitation, suitable enzymes such as horseradish peroxidase, alkaline phosphatase, (3-galactosidase, or acetylcholinesterase; members of a binding pair that are capable of forming complexes such as streptavidin/biotin, avidin biotin or an antigen/antibody complex including, for example, rabbit IgG and anti-rabbit IgG; fluorophores such as umbelliferone, fluorescein, fluorescein isothiocyanate, rhodamine, tetramethyl rhodamine, eosin, green fluorescent protein, erythrosin, coumarin, methyl coumarin, pyrene, malachite green, stilbene, lucifer yellow, Cascade Blue™, Texas Red, dichlorotriazinylamine fluorescein, dansyl chloride, phycoerythrin, fluorescent lanthanide complexes such as those including Europium and Terbium, Cy3, Cy5, molecular beacons and fluorescent derivatives thereof, as well as others known in the art as described, for example, in Principles of Fluorescence Spectroscopy, Joseph R. Lakowicz (Editor), Plenum Pub Corp, 2nd edition (July 1999) and the 6^{th} Edition of the Molecular Probes Handbook by Richard P. Hoagland; a luminescent material such as luminol; light scattering or plasmon resonant materials such as gold or silver particles or quantum dots; or radioactive material include ¹⁴C, ¹²³I, ¹²⁴I, ¹²⁵I, ¹³¹I, Tc99m, ³⁵S or ³H.

Examples of labels include, but are not limited to, chromophores, fluorescent moieties, enzymes, antigens, heavy metal, magnetic probes, dyes, phosphorescent groups, radioactive materials, chemiluminescent moieties, scattering or fluorescent nanoparticles, Raman signal generating moieties, and electrochemical detection moieties. Genotyping using a microarray can be performed using any of a variety of methods, means and variations thereof for carrying out array-genotyping analysis.

Furthermore, backbone labels are nucleic acid stains that bind nucleic acid molecules in a sequence independent manner. Examples include intercalating dyes such as phenanthridines and acridines (e.g., ethidium bromide, propidium iodide, hexidium iodide, dihydroethidium, ethidium homodimer-1 and -2, ethidium monoazide, and ACMA); some minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS751, and hydroxystilbamidine. All of the aforementioned nucleic acid stains are commercially available from suppliers such as Molecular Probes, Inc. Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red).

Therapeutic Polypeptides

As will not be apparent, DL-NAMs can be linked with any therapeutic compound(s) including polypeptides.

Thus, in another aspect of the invention, the therapeutic capable agent is a bioactive protein or peptide. Examples of such bioactive protein or peptides include a cell modulating peptide, a chemotactic peptide, an anticoagulant peptide, an antithrombotic peptide, an anti-tumor peptide, an anti-infectious peptide, a growth potentiating peptide, and an anti-inflammatory peptide. Examples of proteins include antibodies, enzymes, steroids, growth hormone and growth hormone-releasing hormone, gonadotropin-releasing hormone, and its agonist and antagonist analogues, somatostatin and its analogues, gonadotropins such as luteinizing hormone and follicle-stimulating hormone, peptide T, thyrocalcitonin, parathyroid hormone, glucagon, vasopressin, oxytocin, angiotensin I and II, bradykinin, kallidin, adrenocorticotropic hormone, thyroid stimulating hormone, insulin, glucagon and the numerous analogues and congeners of the foregoing molecules. The therapeutic agents may be selected from insulin, antigens selected from the group consisting of MMR (mumps, measles and rubella) vaccine, typhoid vaccine, hepatitis A vaccine, hepatitis B vaccine, herpes simplex virus, bacterial toxoids, cholera toxin B-subunit, influenza vaccine virus, bordetela pertussis virus, vaccinia virus, adenovirus, canary pox, polio vaccine virus, plasmodium falciparum, bacillus calmette geurin (BCG), klebsiella pneumoniae, HIV envelop glycoproteins and cytokins and other agents selected from the group consisting of bovine somatropin (sometimes referred to as BST), estrogens, androgens, insulin growth factors (sometimes referred to as IGF), interleukin I, interleukin II and cytokins. Three such cytokins are interferon-a, interferon-b and tuftsin.

In one embodiment a cell modulating peptide is selected from the group consisting of an anti-integrin antibody fragment, a cadherin binding peptide, a bone morphogenic protein fragment, and an integrin binding peptide. Preferably the cell modulating peptide is a integrin binding peptide which is selected from the group consisting of RGDC (SEQ ID NO: 105), RGEC (SEQ ID NO: 106), RGDT (SEQ ID NO: 107), DGEA (SEQ ID NO: 108), DGEAGC (SEQ ID NO: 109), EPRGDNYR (SEQ ID NO: 110), RGDS (SEQ ID NO: 111), EILDV (SEQ ID NO: 112), REDV (SEQ ID NO: 113), YIGSR (SEQ ID NO: 114), SIKVAV (SEQ ID NO: 115), RGD, RGDV (SEQ ID NO: 116), HRNRKGV (SEQ ID NO: 117), KKGHV (SEQ ID NO: 118), XPQPNPSPASPVVVGGGASLPEFXY (SEQ ID NO: 119), and ASPVVVGGGASLPEFX (SEQ ID NO: 120). The peptides also may be any functionally active fragment of the proteins disclosed herein as being bioactive molecules useful according to the invention. In another embodiment the chemotactic peptide is selected from the group consisting of functionally active fragments of collagen, fibronectin, laminin, and proteoglycan. In yet another embodiment the anti-tumor peptide is selected from the group consisting of functionally active fragments of protein anti-tumor agents. The anti-infectious peptide is selected from the group consisting of functionally active fragments of the protein anti-infectious agents according to another embodiment. In another embodiment the growth potentiating peptide is selected from the group consisting of functionally active fragments of PDGF, EGF, FGF, TGF, NGF, CNTF, GDNF, and type I collagen related peptides. According to another embodiment the anti-inflammatory peptide is selected from the group consisting of functionally active fragments of anti-inflammatory agents.

Other bioactive peptides useful according to the invention may be identified through the use of synthetic peptide combinatorial libraries such as those disclosed in Houghton et al., Biotechniques, 13(3):412-421 (1992) and Houghton et al., Nature, 354:84-86 (1991) or using phage display procedures such as those described in Hart, et al., J. Biol. Chem. 269:12468 (1994). Hart et al. report a filamentous phage display library for identifying novel peptide ligands for mammalian cell receptors. In general, phage display libraries using, e.g., M13 or fd phage, are prepared using conventional procedures such as those described in the foregoing reference. The libraries display inserts containing from 4 to 80 amino acid residues. The inserts optionally represent a completely degenerate or a biased array of peptides. Ligands that bind selectively to a specific molecule such as a cell surface receptor are obtained by selecting those phages which express on their surface a ligand that binds to the specific molecule. Ligands that possess a desired biological activity can be screened in known biological activity assays and selected on that basis. These phages then are subjected to several cycles of reselection to identify the peptide-expressing phages that have the most useful characteristics. Typically, phages that exhibit the binding characteristics (e.g., highest binding affinity or cell stimulatory activity) are further characterized by nucleic acid analysis to identify the particular amino acid sequences of the peptides expressed on the phage surface and the optimum length of the expressed peptide to achieve optimum biological activity. Alternatively, such peptides can be selected from combinatorial libraries of peptides containing one or more amino acids. Such libraries can further be synthesized which contain non-peptide synthetic moieties which are less subject to enzymatic degradation compared to their naturally-occurring counterparts. U.S. Pat. No. 5,591,646 discloses methods and apparatuses for biomolecular libraries which are useful for screening and identifying bioactive peptides. Methods for screening peptides libraries are also disclosed in U.S. Pat. No. 5,565,325.

Nucleic Acid Delivery

In yet another aspect of the invention, the dendrimer (DL-NAM) is linked to therapeutic nucleic acids, including linear or branched nucleic acids, genes/antigenes, nucleic acid vectors (e.g., plasmid or viral vectors or linear nucleic acid sequences), all of which are delivered into a cell or subject utilizing DL-NAMs which can be “loaded” with one or more therapeutic “payloads”.

Thus in some embodiments, the dendrimer structures are used in method of effecting transfection or genetic modification of a cell. One central aspect of the dendrimer structures are anisotropic and multivalent. Such DL-NAMs can be administered to a cell or subject using methods known in the art for delivery of nucleic acids (e.g., plasmids or viral vectors), such as disclosed in U.S. Pat. No. 6,946,448; 6,893,664; 6,821,955; 6,689,757; 6,562,801; 6,951,755; 6,841,540; 6,818,213 or 6,649,407.

In one embodiment, DL-NAMs are comprised of one or more biologically active agents to be delivered alone or in combination with another compound to a cell or subject. In order to use DL-NAMs as scaffolding for multi-functional modules (e.g., bioactive agents such as peptides), the nucleic acids comprising DL-NAMs can be functionalized with the desired bioactive agent. Nucleic acid molecules have been conjugated with many chemical moieties, thus effectively linking diverse chemical functionalities (Zhu et al., J Am Chem Soc 125, 10178 (2003)

In one example, DNA is amine-modified at the commercial synthesis stage. Peptides are synthesized with an extra Cys at their C-terminal. Furthermore, many homo- and hetero-bi-functional crosslinkers can be used for protein nucleic acid conjugation. Examples of some common cross-linkers include succinimdyl 4-(N-maleimidomethyl) cyclohexane-1-carboxylate (SMCC), which is widely employed for protein-protein and protein-oligonucleotide conjugation between an NH₄⁺ group and a SH— group. SMCC has n NHS-ester and a maleimide group, which result in primary amine and sulfhydryl reactivity. The cyclohexane bridge makes the maleimide group extra stable (See, Example below).

In some embodiments, DL-NAMs can comprise nucleic acids encoding therapeutic products. Nonlimiting examples of such nucleic acids include ones encoding interferon, interleukin, erythropoietin, granulocyte-colony stimulating factor (GCSF), stem cell factor (SCI:), leptin (OB protein), interferon (alpha, beta, gamma), ciprofloxacin, amoxycillin, lactobacillus, cefotaxime, levofloxacin, cefipime, mebendazole, ampicillin, lactobacillus, cloxacillin, norfloxacin, timidazole, cefpodoxime, proxctil, azithromycin, gatifloxacin, roxithromycin, cephalosporin, anti-thrombogenics, aspirin, ticlopidine, sulfinpyrazone, heparin, warfarin, growth factors, differentiation factors, hepatocyte stimulating factor, plasmacytoma growth factor, brain derived neurotrophic factor (BDNF), glial derived neurotrophic factor (GDNF), neurotrophic factor 3 (NT3), fibroblast growth factor (FGF), transforming growth factor (TGF), platelet transforming growth factor, milk growth factor, endothelial growth factors (EGF), endothelial cell-derived growth factors (ECDGF), alpha-endothelial growth factors, beta-endothelial growth factor, neurotrophic growth factor, nerve growth factor (NGF), vascular endothelial growth factor (VEGF), 4-1 BB receptor (4-1BBR), TRAIL (TNF-related apoptosis inducing ligand), artemin (GFRalpha3-RET ligand), BCA-1 (B cell-attracting chemokinel), B lymphocyte chemoattractant (BLC), B cell maturation protein (BCMA), brain-derived neurotrophic factor (BDNF), bone growth factor such as osteoprotegerin (OPG), bone-derived growth factor, megakaryocyte derived growth factor (MGDF), keratinocyte growth factor (KGF), thrombopoietin, platelet-derived growth factor (PGDF), megakaryocyte derived growth factor (MGDF), keratinocyte growth factor (KGF), platelet-derived growth factor (PGDF), bone morphogenetic protein 2 (BMP2), BRAK, C-10, Cardiotrophin 1 (CT1), CCR8, anti-inflammatory: paracetamol, salsalate, diflunisal, mefenamic acid, diclofenac, piroxicam, ketoprofen, dipyrone, acetylsalicylic acid, antimicrobials amoxicillin, ampicillin, cephalosporins, erythromycin, tetracyclines, penicillins, trimethprim-sulfamethoxazole, quniolones, amoxicillin, clavulanatf, azithromycin, clarithromycin, anti-cancer drugs aliteretinoin, altertamine, anastrozole, azathioprine, bicalutamide, busulfan, capecitabine, carboplatin, cisplatin, cyclophosphamide, cytarabine, doxorubicin, epirubicin, etoposide, exemestane, vincristine, vinorelbine, hormones, thyroid stimulating hormone (TSH), sex hormone binding globulin (SHBG), prolactin, luteotropic hormone (LTH), lactogenic hormone, parathyroid hormone (PTH), melanin concentrating hormone (MCH), luteinizing hormone (LHb), growth hormone (HGH), follicle stimulating hormone (FSHb), haloperidol, indomethacin, doxorubicin, epirubicin, amphotericin B, Taxol, cyclophosphamide, cisplatin, methotrexate, pyrene, amphotericin B, anti-dyskinesia agents, Alzheimer vaccine, antiparkinson agents, ions, edetic acid, nutrients, glucocorticoids, heparin, anticoagulation agents, anti-virus agents, anti-HIV agents, polyamine, histamine and derivatives thereof, cystineamine and derivatives thereof, diphenhydramine and derivatives, orphenadrine and derivatives, muscarinic antagonist, phenoxybenzamine and derivatives thereof, protein A, streptavidin, amino acid, beta-galactosidase, methylene blue, protein kinases, beta-amyloid, lipopolysaccharides, eukaryotic initiation factor-4G, tumor necrosis factor (TNF), tumor necrosis factor-binding protein (TNF-bp), interleukin-1 (to 18) receptor antagonist (IL-Ira), granulocyte macrophage colony stimulating factor (GM-CSF), novel erythropoiesis stimulating protein (NESP), thrombopoietin, tissue plasminogen activator (TPA), urokinase, streptokinase, kallikrein, insulin, steroid, acetylsalicylic acid, acetaminophen, analgesic, anti-tumor preparation, anti-cancer preparation, anti-proliferative preparation or pro-apoptotic preparation.

In some aspects DL-NAMs can be linked to a plasmid or viral vector which itself encodes a therapeutic gene. Examples of such plasmid or viral vectors include adenoviral vectors, adenoviral associated vectors, retroviral vectors, and/or eukaryotic cell plasmid vectors FIG. 19, which can further encode any therapeutic gene of interest. In various embodiments, a DL-NAM can be linked to one or more desired nucleic acid for delivery to a target cell. Examples of such nucleic acids include genes and antigenes, siRNA, RNAi, nucleic acids.

In various embodiments, DL-NAMs are comprised of one or more targeting moieties, in addition to a therapeutic payload (e.g., genes or antigenes). Targeting moieties are disclosed herein and known in the art, and result in enhanced cellular uptake and release of a therapeutic payload. Furthermore, based on the inherent properties of DL-NAMs, there is reduced toxicity and adverse effects associated with viral delivery vectors. Moreover, DL-NAMs provide modular/multivalent functionality, where for example, targeting and therapeutic (as well as detectable and selectable) compounds can be linked to a particular DL-NAM (FIGS. 18-20).

Delivery can be to eukaryotic or prokaryotic cells. Furthermore, delivery can be to mammalian cells or animals. Furthermore, delivery can be to species of animals including but not limited to simian, human, murine, bovine, equine, bird, reptile or insects.

In yet other aspects of the invention, one or more vectors each encoding a different therapeutic capable agent delivered to cells or tissue via DL-NAMs of the invention. Such delivery can be of plasmid vectors that afford endogenous control via promoters which are sensitive to a physiological signal such as hypoxia or glucose elevation. Furthermore, such plasmid vectors can afford exogenous control systems for gene expression controlled from without the cell, for example, by administering a small molecule drug. Examples include tetracycline, doxycycline, ecdysone and its analogs, RU486, chemical dimerizers such as rapamycin and its analogs, etc.

In an alternative aspect of the invention, DL-NAMs can deliver one or more drug, such as those described herein, where the device is functionalized by linking nucleic acid building blocks to a therapeutic drug (e.g., small molecule drug).

In some embodiments, DL-NAMs are linked to vectors, such as derivatives of SV-40, adenovirus, retrovirus-derived DNA sequences and shuttle vectors derived from combinations of functional mammalian vectors and functional plasmids and phage DNA. Eukaryotic expression vectors are well known, e.g. such as those described by P J Southern and P Berg, J Mol Appl Genet. 1:327-341 (1982); Subramini et al., Mol. Cell. Biol. 1:854-864 (1981), Kaufmann and Sharp, J. Mol. Biol. 159:601-621 (1982); Scahill et al., PNAS USA 80:4654-4659 (1983) and Urlaub and Chasin PNAS USA 77:4216-4220 (1980), which are hereby incorporated by reference. The vector used in one or methods disclosed herein may be a viral vector, preferably a retroviral vector. Replication deficient adenoviruses are preferred. For example, a “single gene vector” in which the structural genes of a retrovirus are replaced by a single gene of interest, under the control of the viral regulatory sequences contained in the long terminal repeat, may be used, e.g. Moloney murine leukemia virus (MoMulV), the Harvey murine sarcoma virus (HaMuSV), murine mammary tumor virus (MuMTV) and the murine myeloproliferative sarcoma virus (MuMPSV), and avian retroviruses such as reticuloendotheliosis virus (Rev) and Rous Sarcoma Virus (RSV), as described by Eglitis and Andersen, BioTechniques 6(7):608-614 (1988), which is hereby incorporated by reference.

Recombinant retroviral vectors into which multiple genes may be introduced may also be used with the matrixes or methods of the invention. As described by Eglitis and Andersen, above, vectors with internal promoters containing a cDNA under the regulation of an independent promoter, e.g. SAX vector derived from N2 vector with a selectable marker (noe.sup.R) into which the cDNA for human adenosine deaminase (hADA) has been inserted with its own regulatory sequences, the early promoter from SV40 virus (SV40) may be designed and used in accordance with methods disclosed herein or as known in the art.

In some aspects of the invention, the vectors comprising recombinant nucleic acid molecules are first introduced (e.g., transfected) into cells, which cells are deposited in the matrixes of the invention. For example, the vectors comprising the recombinant nucleic acid molecule are incorporated, i.e. infected, into the BM-MNCs by plating ˜5e5 BM-MNCs over vector-producing cells for 18-24 hours, as described by Eglitis and Andersen BioTechniques 6(7):608-614 (1988), which is hereby incorporated by reference, and subsequently said cells are deposited into the reservoir portion of the device.

In mammalian host cells, a number of viral-based expression systems can be utilized. In cases where an adenovirus is used as an expression vector, the nucleotide sequence of interest (e.g., encoding a therapeutic capable agent) can be ligated to an adenovirus transcription or translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the AQP1 gene product in infected hosts. (See e.g., Logan & Shenk, Proc. Natl. Acad. Sci. USA 8 1:3655-3659 (1984)).

In one embodiment, DL-NAMs are utilized in a cell culture to deliver a particular agent and to monitor the effects of such an agent on cells or tissue cultures. Generally, the DL-NAMs can be utilized in any method of the priros art where it is desired to transfect or genetically modify a cell. For example, in a method of screening different agents to determine the mechanisms, by which such compounds induce cell differentiation, e.g., such as in studying effects on stem cells. Methods of utilizing cell and tissue culture are known in the art, such as disclosed in U.S. Pat. Nos. 7,008,634 (using cell growth substrates with tethered cell growth effector molecules); 6,972,195 (culturing potentially regenerative cells and functional tissue organs in vitro); 6,982,168 or 6,962,980 (using cell culture to assay compounds for treating cancer); 6,902,881 (culturing techniques to identify substances that mediate cell differentiation); 6,855,504 (culturing techniques for toxicology screening); or 6,846,625 (identifying validated target drug development using cell culture techniques). The DL-NAMS of the invention are readily adaptable to such cell culturing techniques as would be evident to one of ordinary skill in the art.

### Drugs of Use in the Invention

In some aspects of the invention, DL-NAMs of the invention are linked to one or more drugs (e.g., FIGS. 18 and 20). DL-NAMs of the invention provide modular/anisotropic sites for linkage to one or more drugs. Furthermore, particular DL-NAMs can be tailored to increase the number of one drug or the number of a particular drug, through iterative rounds of conjugation. As such, different DL-NAMs comprise different dosages. Of course, dosage can also be controlled by temporal regulation (e.g., number of administration, such as systemically, locally, epidermally, muscular injection, etc., as well as frequency of administration in a given period of time.

Thus, the methods and compositions of the invention include the study and use of drugs, e.g., insulin sensitizers, and include performing association studies for determining genotypic and/or phenotypic traits associated with responsiveness to drugs, e.g., insulin sensitizers, screening individuals for predisposition to response to drugs, e.g., insulin sensitizers, e.g., adverse response, and/or administering or not administering drugs, e.g., insulin sensitizers to the individual based on such screening. The following relevant sections describe certain drugs of use in embodiments of the invention. Thus, in various embodiments, one or more of such drugs can be conjugated to DL-NAMs for delivery to a cell or subject.

Insulin Sensitizers

One class of drugs included in certain embodiments of the invention is an insulin sensitizer. The term “insulin sensitizer,” or “insulin sensitizing agent,” as used herein, refers to any agent capable of enhancing either secretion of or, more typically, tissue sensitivity to, insulin. Non-exclusive examples of insulin sensitizers include metformin, sulfonylureas, alpha glucosidase inhibitors and PPAR modulators, including thiazolidinediones. Further examples of insulin sensitizers are described below.

The thiazolidinediones are examples of PPAR modulators, which are one class of insulin sensitizers. The term “PPAR modulator,” as used herein, refers to peroxisome proliferator-activated receptor agonists, partial agonists, and antagonists. The modulator may, selectively or preferentially, affect PPAR alpha, PPAR gamma, or both receptors. Typically, the modulator increases insulin sensitivity. According to one aspect, the modulator is a PPAR gamma agonist. One PPAR gamma agonist used in embodiments of the invention is 5-[{6-(2-fluorobenzyl)oxy-2-naphthyl}methyl]-2,4-thiazolidinedione; (MCC-555 or “netoglitazone”).

Insulin Sensitizers—PPAR Modulators

One class of insulin sensitizers of the invention is PPAR modulators, and in particular PPAR-gamma modulators, e.g., PPAR-gamma agonists. PPAR modulators include the PPAR-alpha, PPAR-delta (also called PPAR-beta), and PPAR-gamma agonists. Especially useful are the thiazolidinediones (TZDs), which were developed in the 70's and 80s by screening newly synthesized compounds for their ability to lower blood glucose in diabetic rodents. Three molecules from this class, troglitazone, rosiglitazone, and pioglitazone, were ultimately approved for the treatment of patients with Type II diabetes. Although these compounds were developed without an understanding of their molecular mechanism of action, by the early 90s evidence began to accumulate linking the thiazolidinediones to the nuclear receptor PPAR-gamma. It was ultimately demonstrated that these molecules were high affinity ligands of PPAR-gamma and that they increased transcriptional activity of the receptor. Without wishing to be bound by theory, multiple lines of evidence now indicate that the antidiabetic activities of the thiazolidinediones are mediated by their direct interaction with the receptor and the subsequent modulation of PPAR-gamma target gene expression.

Thiazolidinediones of use in the methods of the invention include: (1) rosiglitazone; (2) pioglitazone; (3) troglitazone; (4) netoglitazone (also known as MCC-555 or isaglitazone or neoglitazone); and (5) 5-BTZD.

Other PPAR modulators of use in the invention include modulators that have recently been the subject of clinical trials: (1) Muraglitazar (PPAR gamma and alpha agonist, Bristol-Myers/Merck); (2) Galida tesaglitazar (PPAR gamma and alpha agonist, AstraZeneca); (3) 677954 (PPAR gamma, alpha, and delta agonist, GlaxoSmithKline); (4) MBX-102 (PPAR gamma partial agonist/antagonist, Metabolex); (5) T131 (PPAR gamma selective modulator, Tularik/Amgen); (6) LY818 (PPAR gamma and alpha partial agonist, Eli Lilly/Ligand); (7) LY929 (PPAR gamma and alpha agonist, Eli Lilly/Ligand); and (8) PLX204 (PPAR gamma, alpha, and delta agonist, Plexxikon). See, e.g., BioCentury, Jun. 14, 2004. Further PPAR modulators include LY 519818, L-783483, L-165461, and L-165041.

Additionally, the non-thiazolidinediones that act as insulin-sensitizing agents include, but are not limited to: (1) JT-501 (JTT 501, PNU-1827, PNU-7,6-MET-0096, or PNU 182716: 4-(4-(2-(5-methyl-2-phenyl-oxazol-4-yl)ethoxy)benzyl)isoxazolidine-3,5-dione; (2) KRP-297 (5-(2,4-dioxothiazolidin-5-ylmethyl)-2-methoxy-N-(4-(tri-fluoromethyl) benzyl)benzamide or 5-((2,4-dioxo-5-thiazolidinyl)methyl)-2-methoxy-N-((4-(trifluoromethyl)phenyl) methyl)benzamide); and (3) Farglitazar (L-tyrosine, N-(2-benzoylphenyl)-o-(2-(5-methyl-2-phenyl-4-oxazolyl)ethyl) or N-(2-benzoylphenyl)-O-(2-(5-methyl-2-phenyl-4-oxazolyl)ethyl)-L-tyrosine, or (S)-2-(2-benzoylphenylamino)-3-(4-12-(5-methyl-2-phenyl-2-oxazo-4-yl)ethoxyphenyl)propionic acid, or GW2570 or G1-262570).

Other agents have also been shown to have PPAR modulator activity such as PPAR-gamma, SPPAR-gamma, and/or PPAR-alpha/delta agonist activity. Examples are: (1) AD 5075 (5-(4-(2-hydroxy-2-(5-methyl-2-phenyloxazol-4-yl)ethoxy)benzyl)-thiazolidine-2,4-dione); (2) R 119702 (or Cl 1037 or CS 011); (3) CLX-0940 (peroxisome proliferator-activated receptor alpha agonist/peroxisome proliferator-activated receptor gamma agonist); (4) LR-90 (2,5,5-tris(4-chlorophenyl)-1,3-dioxane-2-carboxylic acid, PPAR alpha/gamma agonist); (5) CLX-0921 (PPAR gamma agonist); (6) CGP-52608 (PPAR agonist); (7) GW-409890 (PPAR agonist); (8) GW-7845 (2((S)-1-carboxy-2-(4-(2-(5-methyl-2-phenyl-oxazol-4-yl)-ethoxy)-phenyl)-ethyamino)-benzoic acid methyl ester, PPAR agonist); (9) L-764406 (2-benzenesulphonylmethyl-3-chloroquinoxaline, PPAR agonist); (10) LG-101280 (PPAR agonist); (11) LM-4156 (PPAR agonist); (12) Risarestat (CT-112, (+)-5-(3-ethoxy-4-(pentyloxy)phenyl-2,4-thiazolidinedione aldose reductase inhibitor); (13) YM 440 (PPAR agonist); (14) AR-H049020 (PPAR agonist); (15) GW 0072 ((+)-(2S,5S)-4-(4-(5-((dibenzy carbomoyl)methyl)-2-heptlyl-4-oxothiazolidin-3-yl butyl)benzoic acid); (16) GW 409544 (GW-544 or GW-409544); (17) NN 2344 (DRF 2593); (18) NN 622 (DRF 2725); (19) AR-H039242 (AZ-242); (20) GW 9820 (fibrate); (21) GW 1929 (N-(2-benzoylphenyl)-O-(2-(methyl-2-pyridinylamino) ethyl)-L-tyrosine, known as GW 2331, PPAR agonist); (22) SB 219994 ((S)-4-(2-(2-benzoxazolylmethylamino)ethoxy)-alpha-(2,2,2-trifluoroethoxy)benzenepropanoic acid or 3-(4-(2-(N-(2-benzoxazolyl)-N-methylamino)ethoxy)phenyl)-2 (S)-(2,2,2-trifluoroethoxy)propionic acid or benzenepropanoic acid, 4-(2-(2-benzoxazolylmethylamino)ethoxy)-alpha-(2,2,2-trifluoroethox-y)-, (alpha S)-, PPAR alpha/gamma agonist); (23) L-796449 (PPAR alpha/gamma agonist); (24) Fenofibrate (propanoic acid, 2-[4-(4-chlorobenzoyl)phenoxy]-2-methyl-, 1-methylethyl ester, known as TRICOR, LIPCOR, LIPANTIL, LIPIDIL MICRO PPAR alpha agonist); (25) GW-9578 (PPAR alpha agonist); (26) GW-2433 (PPAR alpha/gamma agonist); (27) GW-0207 (PPAR gamma agonist); (28) LG-100641 (PPAR gamma agonist); (29) LY-300512 (PPAR gamma agonist); (30) NID525-209 (NID-525); (31) VDO-52 (VDO-52); (32) LG 100754 (peroxisome proliferator-activated receptor agonist); (33) LY-510929 (peroxisome proliferator-activated receptor agonist); u(34) bexarotene (4-(1-(3,5,5,8,8-pentamethyl-5,6,7,8-tetrahydro-2-naphthalenyl)ethenyl)benzoic acid, known as TARGRETIN, TARGRETYN, TARGREXIN; also known as LGD 1069, LG 100069, LG 1069, LDG 1069, LG 69, RO 264455); and (35) GW-1536 (PPAR alpha/gamma agonist).

In some aspects of the invention, radioisotopes can be delivered via the implantable device of the invention. For example, it is well known in the art that various methods of radionuclide therapy can be used for the treatment of cancer and other pathological conditions, as described, e.g., in Harbert, “Nuclear Medicine Therapy”, New York, Thieme Medical Publishers, 1987, pp. 1-340. A clinician experienced in these procedures will readily be able to adapt the implantable device described herein to such procedures to mitigate or treat disease amenable to radioisotope therapy thereof.

In some aspects the radio isotopes include but are not limited to isotopes and salts of isotopes with short half life: such as Y-90, P-32, 1-131, Au 198. Therefore in one aspect of the invention, the implantable device can be utilized to deliver radioisotopes.

In some embodiments, DL-NAMs are linked to antibodies alone or antibodies conjugated to radioisotopes. Therefore, antibodies can be directed to particular/specific cellular epitope, thereby functioning as a targeting moiety. For example, many cellular epitopes are known in the art that are differentially expressed in different cell types (e.g., specific organs, cancer/versus normal, diseased/versus non-diseased). Therefore, in various embodiments, DL-NAMs can be targeted to particular cell types and to deliver a therapeutic drug.

It is also well known that radioisotopes, drugs, and toxins can be conjugated to antibodies or antibody fragments which specifically bind to markers which are produced by or associated with cancer cells, and that such antibody conjugates can be used to target the radioisotopes, drugs or toxins to tumor sites to enhance their therapeutic efficacy and minimize side effects. Examples of these agents and methods are reviewed in Wawrzynczak and Thorpe (in Introduction to the Cellular and Molecular Biology of Cancer, L. M. Franks and N. M. Teich, eds, Chapter 18, pp. 378-410, Oxford University Press, Oxford, 1986), in Immunoconjugates. Antibody Conjugates in Radioimaging and Therapy of Cancer (C.-W. Vogel, ed., 3-300, Oxford University Press, New York, 1987), in Dillman, R. O. (CRC Critical Reviews in Oncology/Hematology 1:357, CRC Press, Inc., 1984), in Pastan et al. (Cell 47:641, 1986), in Vitetta et al. (Science 238:1098-1104, 1987) and in Brady et al. (Int. J. Rad. Oncol. Biol. Phys. 13:1535-1544, 1987). Other examples of the use of immunoconjugates for cancer and other forms of therapy have been disclosed, inter alia, in Goldenberg, U.S. Pat. Nos. 4,331,647, 4,348,376, 4,361,544, 4,468,457, 4,444,744, 4,460,459, 4,460,561 and 4,624,846, and in Rowland, U.S. Pat. No. 4,046,722, Rodwell et al., U.S. Pat. No. 4,671,958, and Shih et al., U.S. Pat. No. 4,699,784, the disclosures of all of which are incorporated herein in their entireties by reference.

Other thiazolidinedione and non-thiazolidinedione insulin sensitizers of use in the invention are described in, e.g., Leff and Reed (2002) Curr. Med. Chem. Imun., Endoc., & Metab. Agents 2:33-47; Reginato et al. (1998) J. Biol. Chem., 278 32679-32654; Way et al. (2001) J. Biol. Chem. 276 25651-25653; Shiraki et al. (2005) JBC Papers in Press, published on Feb. 4, 2005, as Manuscript M500901200, and U.S. Pat. Nos. 4,703,052; 6,008,237; 5,594,016; 6,838,442; 6,329,423; 5,965,589; 6,677,363; 4,572,912; 4,287,200; 4,340,605; 4,438,141; 4,444,779; 4,572,912; 4,687,777; 4,725,610; 5,232,925; 5,002,953; 5,194,443; 5,260,445; 6,300,363; 6,034,110; and 6,541,493; U.S. Patent Application Publications 2002/0042441; 2004/0198774 and 2003/0045553; EP Patent Nos. 0139421 and 0332332; and PCT Publication Nos. WO 95/35314; WO 00/31055; WO 01/3640, all of which are incorporated by reference herein in their entirety.

Netoglitazone

One thiazolidinedione PPAR modulator for use in the methods of the invention is netoglitazone (5-[{6-(2-fluorobenzyl)oxy-2-naphthyl}methyl]-2,4-thiazolidinedione; MCC-555). Structures and methods of preparation of netoglitazone and various forms of netoglitazone of use in the invention are described in, e.g., U.S. Pat. Nos. 5,594,016; 6,541,493; 6,541,493; 6,838,442; U.S. Patent Application No. 2004/0198774 and 2003045553; PCT Publication Nos. WO 00/31055; WO 01/36401; WO 03/018010, and WO 00/73252; Japanese Patent Unexamined Publication (KOKAI) Nos. (Hei) 6-247945/1994 and (Hei) 10-139768/1998; Japanese Patents 2001172179 and 2003040877; and Reginato et al. (1998) J. Biol. Chem. 273: 32679-32684; all of which are incorporated by reference herein in their entirety.

It has been reported that netoglitazone is more efficacious than pioglitazone and troglitazone in lowering plasma glucose, insulin, and triglyceride levels and that it is about three-fold more potent than rosiglitazone. The activity of netoglitazone appears to be context-specific, as in some cell types it behaves as a full agonist of PPAR-gamma and as a partial agonist or antagonist in others. In addition, it appears to modulate PPAR-alpha and delta as well. See, e.g., U.S. Patent Application Publication No. 2004/0198774.

Forms of Drugs

Some compounds useful in the invention, including the TZD PPAR modulators such as netoglitazone, may have one or more asymmetric carbon atoms in their structure. In addition, stereochemically pure isomeric forms of the compounds as well as their racemates can also be delivered using one or more matrix disclosed herein. Stereochemically pure isomeric forms may be obtained by the application of art known principles. Diastereoisomers may be separated by physical separation methods such as fractional crystallization and chromatographic techniques, and enantiomers may be separated from each other by the selective crystallization of the diastereomeric salts with optically active acids or bases or by chiral chromatography. Pure stereoisomers may also be prepared synthetically from appropriate stereochemically pure starting materials, or by using stereospecific reactions.

Some compounds useful in the invention may have various individual isomers, such as trans and cis, and various alpha and beta attachments (below and above the plane of the drawing). In addition, where the processes for the preparation of the compounds according to the invention give rise to mixture of stereoisomers, these isomers may be separated by conventional techniques such as preparative chromatography. The compounds may be prepared as a single stereoisomer or in racemic form as a mixture of some possible stereoisomers. The non-racemic forms may be obtained by either synthesis or resolution. The compounds may, for example, be resolved into their components enantiomers by standard techniques, such as the formation of diastereomeric pairs by salt formation. The compounds may also be resolved by covalent linkage to a chiral auxiliary, followed by chromatographic separation and/or crystallographic separation, and removal of the chiral auxiliary. Alternatively, the compounds may be resolved using chiral chromatography. Unless otherwise noted the scope of the bioactive agents, that can be included in the matrix(es) disclosed herein, is intended to cover all such isomers or stereoisomers per se, as well as mixtures of cis and trans isomers, mixtures of diastereomers and racemic mixtures of enantiomers (optical isomers) as well.

In addition, compounds to be delivered by or included in the matrixes of the invention may be prepared in various polymorphic forms. For example, insulin sensitizers of use in the invention can occur in polymorphic forms, and any or all of the polymorphic forms of these insulin sensitizers are contemplated for use in the invention. Polymorphism in drugs may alter the stability, solubility and dissolution rate of the drug and result in different therapeutic efficacy of the different polymorphic forms of a given drug. The term polymorphism is intended to include different physical forms, crystal forms, and crystalline/liquid crystalline/non-crystalline (amorphous) forms. Polymorphism of compounds of therapeutic use has is significant, as evidenced by the observations that many antibiotics, antibacterials, tranquilizers etc., exhibit polymorphism and some/one of the polymorphic forms of a given drug may exhibit superior bioavailability and consequently show much higher activity compared to other polymorphs. For example, Sertraline, Frentizole, Ranitidine, Sulfathiazole, and Indomethacine are some of the pharmaceuticals that exhibit polymorphism.

Some embodiments of the invention include the use of netoglitazone in one of its polymorphic forms. Netoglitazone can be prepared in various polymorphic forms. Any polymorphic forms of netoglitazone known in the art may be used in the methods of the invention, either separately or in combination. Thus, the methods of the invention include association studies using any or all of the polymorphic forms of netoglitazone, as well as screening and treatment using any or all of the polymorphic forms of netoglitazone, compositions and kits based on these forms, and the like.

Polymorphic forms of netoglitazone include the A, B, C, D, E and amorphous crystal forms described in PCT Published Application No. WO 01/36401 and in U.S. Pat. No. 6,541,493; for example, the E form is described in PCT Published Application No. WO 01/36401.

Some of the compounds described herein may exist with different points of attachment of hydrogen coupled with double bond shifts, referred to as tautomers. An example is a carbonyl (e.g. a ketone) and its enol form, often known as keto-enol tautomers. The individual tautomers as well as mixtures thereof are encompassed within the invention.

Prodrugs are compounds that are converted to the claimed compounds as they are being administered to a patient or after they have been administered to a patient. The prodrugs are compounds of this invention, and the active metabolites of the prodrugs are also compounds of the invention.

Other agents useful in the methods of the invention include, but are not limited to:

1. Biguanides, which decrease liver glucose production and increases the uptake of glucose. Examples include metformin such as: (1) 1,1-dimethylbiguanide (e.g., Metformin-DepoMed, Metformin-Biovail Corporation, or METFORMIN GR (metformin gastric retention polymer)); and (2) metformin hydrochloride (N,N-dimethylimidodicarbonimidic diamide monohydrochloride, also known as LA 6023, BMS 207 150, GLUCOPHAGE, or GLUCOPHAGE XR.

2. Alpha-glucosidase inhibitors, which inhibit alpha-glucosidase, and thereby delay the digestion of carbohydrates. The undigested carbohydrates are subsequently broken down in the gut, reducing the post-prandial glucose peak. Examples include, but are not limited to: (1) acarbose (D-glucose, O-4,6-dideoxy-4-(((1S-(1alpha,4alpha,5beta,6alpha))-4,5,6-trihydroxy-3-(hydroxymethyl)-2-cyc-lohexen-1-yl)amino)-alpha-D-glucopyranosyl-(1-4)-O-alpha-D-glucopyranosyl-(1-4)-, also known as AG-5421, Bay-g-542, BAY-g-542, GLUCOBAY, PRECOSE, GLUCOR, PRANDASE, GLUMIDA, or ASCAROSE); (2) Miglitol (3,4,5-piperidinetriol, 1-(2-hydroxyethyl)-2-(hydroxymethyl)-, (2R (2alpha,3beta,4alpha,5beta))- or (2R,3R,4R,5S)-1-(2-hydroxyethyl)-2-(hydroxymethyl-3,4,5-piperidinetriol, also known as BAY 1099, BAY M 1099, BAY-m-1099, BAYGLITOL, DIASTABOL, GLYSET, MIGLIBAY, MITOLBAY, PLUMAROL); (3) CKD-711 (0-4-deoxy-4-((2,3-epoxy-3-hydroxymethyl-4,5,6-trihydro-xycyclohexane-1-yl)amino)-alpha-b-glucopyranosyl-(1-4)-alpha-D-glucopyran-osyl-(1-4)-D-glucopyranose); (4) emiglitate (4-(2-((2R,3R,4R,5S)-3,4,5-trihydroxy-2-(hydroxymethyl)-1-piperidinyl)ethoxy) benzoic acid ethyl ester, also known as BAY o 1248 or MKC 542); (5) MOR14 (3,4,5-piperidinetriol, 2-(hydroxymethyl)-1-methyl-, (2R-(2alpha, 3beta, 4alpha, 5beta))-, also known as N-methyldeoxynojirimycin or N-methylmoranoline); and (6) Voglibose (3,4-dideoxy-4-((2-hydroxy-1-(hydroxymethyl)ethyl)amino)-2-C-(hydroxymethyl)-D-epi-inositol or D-epi-lnositol,3,4-dideoxy-4-(2-hydroxy-1-(hydroxymethyl)ethyl)amino)-2-C-(hydroxymethyl)-, also known as A 71100, AO 128, BASEN, GLUSTAT, VOGLISTAT.

3. Insulins include regular or short-acting, intermediate-acting, and long-acting insulins, injectable, non-injectable or inhaled insulin, transdermal insulin, tissue selective insulin, glucophosphokinin (D-chiroinositol), insulin analogues such as insulin molecules with minor differences in the natural amino acid sequence and small molecule mimics of insulin (insulin mimetics), and endosome modulators. Examples include, but are not limited to: (1) Biota; (2) LP 100; (3) (SP-5-21)-oxobis(1-pyrrolidinecarbodithioato-S, S′) vanadium, (4) insulin aspart (human insulin (28B-L-aspartic acid) or B28-Asp-insulin, also known as insulin X14, INA-X14, NOVORAPID, NOVOMIX, or NOVOLOG); (5) insulin detemir (Human 29B-(N-6-(1-oxotetradecyl)-L-lysine)-(1A-21A), (1B-29B)-Insulin or NN 304); (6) insulin lispro (“28B-L-lysine-29B-L-proline human insulin, or Lys (B28), Pro (B29) human insulin analog, also known as lys-pro insulin, LY 275585, HUMALOG, HUMALOG MIX 75/25, or HUMALOG MIX 50/50); (7) insulin glargine (human (A21-glycine, B31-arginine, B32-arginine) insulin HOE 901, also known as LANTUS, OPTISULIN); (8) Insulin Zinc Suspension, extended (Ultralente), also known as HUMULIN U or ULTRALENTE; (9) Insulin Zinc suspension (Lente), a 70% crystalline and 30% amorphous insulin suspension, also known as LENTE ILETIN II, HUMULIN L, or NOVOLIN L; (10) HUMULIN 50/50 (50% isophane insulin and 50% insulin injection); (11) HUMULIN 70/30 (70% isophane insulin NPH and 30% insulin injection), also known as NOVOLIN 70/30, NOVOLIN 70/30 PenFill, NOVOLIN 70/30 Prefilled; (12) insulin isophane suspension such as NPH ILETIN II, NOVOLIN N, NOVOLIN N PenFill, NOVOLIN N Prefilled, HUMULIN N; (13) regular insulin injection such as ILETIN II Regular, NOVOLIN R, VELOSULIN BR, NOVOLIN R PenFill, NOVOLIN R Prefilled, HUMULIN R, or Regular U-500 (Concentrated); (14) ARIAD; (15) LY 197535; (16) L-783281; and (17) TE-17411.

4. Insulin secretion modulators such as (1) glucagon-like peptide-1 (GLP-1) and its mimetics; (2) glucose-insulinotropic peptide (GIP) and its mimetics; (3) exendin and its mimetics; (4)dipeptyl protease (DPP or DPPIV) inhibitors such as (4a) DPP-728 or LAF 237 (2-pyrrolidinecarbonitrile, 1-(((2-((5-cyano-2-pyridinyl)amino)ethyl) amino)acetyl), known as NVP-DPP-728, DPP-728A, LAF-237); (4b) P 3298 or P32/98 (di-(3N-((2S,3S)-2-amino-3-methyl-pentanoyl+1,3-thiazolidine)fumarate); (4c) TSL 225 (tryptophyl-1,2,3,4-tetrahydroisoquinoline-3-carboxyli-c acid); (4d) Valine pyrrolidide (valpyr); (4e) 1-aminoalkylisoquinolinone-4-carboxylates and analogues thereof; (4f) SDZ 272-070 (1-(L-Valyl) pyrrolidine); (4g) TMC-2A, TMC-2B, or TMC-2C; (4h) Dipeptide nitriles (2-cyanopyrrolodides); (41) CD26 inhibitors; and (4j) SDZ 274-444; (5) glucagon antagonists such as AY-279955; and (6) amylin agonists which include, but are not limited to, pramlintide (AC-137, Symlin, tripro-amylin or pramlintide acetate).

Insulin secretagogues, which increase insulin production by stimulating pancreatic beta cells, such as: (1) asmitiglinide ((2 (S)-cis)-octahydro-gamma-oxo-alpha-(phenylmet-hyl)-2H-isoindole-2-butanoic acid, calcium salt, also known as mituglimide calcium hydrate, KAD 1229, or S 21403); (2) Ro 34563; (3) nateglinide (trans-N-((4-(1-methylethyl)cyclohexyl) carbonyl)-D-phenylalanine, also known as A 4166, AY 4166, YM 026, FOX 988, DJN 608, SDZ DJN608, STARLIX, STARSIS, FASTIC, TRAZEC); (4) JTT 608 (trans-4-methyl-gamma-oxocyclohexanebutanoic acid); (5) sulfonylureas such as: (5a) chlorpropamide (1-[(p-chlorophenyl) sulfonyl]-3-propylurea, also known as DIABINESE); (5b) tolazamide (TOLINASE or TOLANASE); (5c) tolbutamide (ORINASE or RASTINON); (5d) glyburide (1-[[p-[2-(5-chloro-o-anisamido) ethyl]phenyl]sulfon-yl]-3-cyclohexylurea, also known as Glibenclamide, DIABETA, MICRONASE, GLYNASE PresTab, or DAONIL); (5e) glipizide (1-cyclohexyl-3-[[p-[2-(5-ethylpyrazinecarboxamido)e-thyl]phenyl]sulfonyl]urea, also known as GLUCOTROL, GLUCOTROL XL, MINODIAB, or GLIBENESE); (5f) glimepiride (1H-pyrrole-1-carboxamide, 3-ethyl-2,5-dihydro-4-m-ethyl-N-[2-[4-[[[[(4-methylcyclohexyl)amino]carbonyl]amino]sulfonyl]phenyl-]ethyl]-2-oxo-, trans-, also known as Hoe-490 or AMARYL); (5g) acetohexamide (DYMELOR); (5h) gliclazide (DIAMICRON); (51) glipentide (STATICUM); (5j) gliquidone (GLURENORM); and (5k) glisolamide (DIABENOR); (6) K+ channel blockers including, but not limited to, meglitinides such as (6a) Repaglinide ((S)-2-ethoxy-4-(2-((3-methyl-1-(2-(1-piperidinyl)phenyl) butyl)amino)-2-oxoethyl)benzoic acid, also known as AGEE 623, AGEE 623 ZW, NN 623, PRANDIN, or NovoNorm); (6b) imidazolines; and (6c) a-2 adrenoceptor antagonists; (7) pituitary adenylate cyclase activating polypeptide (PAcAP); (8) vasoactive intestinal peptide (VIP); (9) amino acid analogs; and (10) glucokinase activators.

Growth Factors such as: (1) insulin-like growth factors (IGF-1, IGF-2); (2) small molecule neurotrophins; (3) somatostatin; (4) growth hormone-releasing peptide (GHRP); (5) growth hormone-releasing factor (GHRF); and (6) human growth hormone fragments. Immunomodulators such as: (1) vaccines; (2) T-cell inhibitors; (3) monoclonal antibodies; (4) interleukin-1 (IL-1) antagonists; and (5) BDNF. Glucose resorption inhibitors such as those described in U.S. Patent Application No. 2003/0045553. Other antidiabetic agents: (1)ᵣHu-Glucagon; (2) DHEA analogs; (3) carnitine palmitoyl transferase (CPT) inhibitors; (4) islet neurogenesis; (5) pancreatic p amyloid inhibitors; and (6) UCP (uncoupling protein)-2 and UCP-3 modulators.

Additional agents of use in the invention include any agents known in the art for treatment of disorder of blood glucose regulations and/or their complications. Such agents include, but are not limited to, cholesterol lowering agents such as (i) HMG-CoA reductase inhibitors (lovastatin, simvastatin and pravastatin, fluvastatin, atorvastatin, rivastatin and other statins), (ii) sequestrants (cholestyramine, colestipol and a dialkylaminoalkyl derivatives of a cross-linked dextran), (iii) nicotinyl alcohol, nicotinic acid or a salt thereof, (iv) PPAR.alpha. agonists such as fenofibric acid derivatives (gemfibrozil, clofibrate, fenofibrate and benzafibrate), (v) inhibitors of cholesterol absorption for example beta-sitosterol and (acyl CoA: cholesterol acyltransferase) inhibitors for example melinamide and (vi) probucol; PPARdelta agonists such as those disclosed in WO97/97/28149; antiobesity compounds such as fenfluramine, dexfenfluramine, phentiramine, sulbitramine, orlistat, neuropeptide Y5 inhibitors, and, β3 adrenergic receptor agonist; and ileal bile acid transporter inhibitors.

Classes of Drugs

Drugs may be classed into mechanistic classes, structural classes, classes based on pharmacological effect, and other classes of drugs that are based on the chemical or biological nature of the drugs, or that are empirically based.

Mechanistic classifications are based on the mechanism of action of drugs, e.g., receptor targets or other targets of the drugs. For example, drugs that primarily act on the autonomic nervous system may be classed as cholinoreceptor-activating drugs, or cholinesterase-inhibiting drugs, or cholinoceptor-blocking drugs, or adrenoceptor-activating drugs, or adrenoceptor-blocking drugs.

However, as is known in the art, often drugs do not have a known target or a precisely defined mechanism, and may be classed according to similarities in other aspects the drugs, such as similarities of the chemical structure that are thought to be important to the action of the drugs. Such similarities include structural components, optical isomerism, crystal structure, and the like.

Drugs may also be classed based on their major pharmacological action, e.g., lipid-lowering drugs, antidepressants, anxiolytics, and the like. The second drug may be placed in the same class as the first drug by in vitro and/or in vivo studies; in some embodiments, action through the same or similar mechanism may be predicted from structural analysis.

In some embodiments, drugs are classified based on their effects in one or more in vitro, cellular, tissue, organ, or animal models. Such effects may be molecular, supramolecular, cellular, tissue, organ, or whole-organism effects, or combinations thereof. In some embodiments, drugs are classified based on their effects in one or more animal models together with associations between genotypes and response in the animal models. For example, drug A may cause response M in a mammal, e.g., a rat, mouse, or primate, of genotype X (e.g., genotype at one or more SNPs), and may cause response N in a primate of genotype Y. If drug B is found to cause response M in a mammal of genotype X and response N in a mammal of genotype Y, then drug B is considered to be in the same class as drug A. It will be appreciated that such classification may be greatly refined based on the number of genetic variations included in the genotype, the number of responses measured, and the like. The animal model allows a much wider range of drugs to be tested, as well as more invasive parameters to be measured as indications of response, and can allow a much more extensive database to be established in a relatively short time, compared to human testing.

In other embodiments, expression profiles for a drug in a model system may be used to classify the drug. For example, all, most, or some of the known drugs of a class of drugs that has an effect in humans (e.g., statins that lower the risk of heart disease) may be tested in an animal model. Animals administered the drug may show consistent profiles of gene expression in response to the drug (e.g., increases in expression of a gene or set of genes related to antiinflammatory activity). Other drugs of other classes may be tested in animal models. The expression profiles associated with the drugs in a particular class may be correlated. A new drug may be assigned to a drug class based on its expression profile in one or more animal models. The associations of one or more drugs in that class between one or more genetic variations and a response to the drug(s) may be used to modulate the use of the new drug, for example, in research (e.g., clinical trials) and/or in the clinical setting.

In some embodiments, a new drug in a class of drugs is first tested in a model, e.g. an animal model, in which other drugs in the class of drugs have been tested, and in which a genotype for the animal is used to predict responses to the new drug. The results of the animal studies can be used to refine predictions for the association between genetic variations and response to a new drug in humans. Animal models may be developed or existing animal models may be used. The animal model can be for a particular physiological, biochemical, or metabolic state, e.g., a disease or pathological state. Healthy or superhealthy states may also be modeled (e.g., decelerated aging).

Drugs may be further put into classes, or into subclasses of the same class, by classifications based on their mode administration (e.g., intravascular, intramuscular, subcutaneous, ocular, inhalation, oral, sublingual, suppository, skin, via pump, and the like), formulation type (e.g., rapid acting, sustained release, enterically coated, etc.), mode of uptake and delivery to site of action, metabolism (e.g., drugs metabolized through Phase I reactions such as oxidation via hepatic microsomal P450 system and subclasses thereof, through oxidation via nonmicrosomal mechanisms and subclasses thereof, through reduction, through hydrolysis and subclasses thereof drugs metabolized through Phase II reactions such as glucoronidation, acetylation, mercapturic acid formation, sulfate conjugation, N-, O-, and S-methylation, trans-sulfuration; and combinations thereof), metabolic products and/or byproducts and their structure and/or function, pharmacokinetics, pharmacodynamics, elimination, and the like

It will be appreciated that these classifications are exemplary only, and that any means of classifying drugs that allows a non-random predictability of the effects of drugs in the class may be used. Further systems of drug classification and specific drugs within each class may be found in the art. See, e.g., Anderson, Philip O.; Knobeni, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins, 2000; Martindale, The Extra Pharmacopoeia, Thirty-Second Edition (The Pharmaceutical Press, London, 1999); all of which are incorporated by reference herein in their entirety.

Any suitable class of drugs for which genotyping and association studies are possible for at least one member of the class may be the subject of the described methods and compositions. Classes include the insulin sensitizers as described herein, e.g., PPAR modulators. Thus, in some embodiments, the invention provides a method for predicting an individual's responsiveness to an insulin sensitizer, e.g., a PPAR modulator based on the individual's genotype and the results of association studies between genotype and responsiveness to another insulin sensitizer, e.g., PPAR modulator. In some embodiments, the prediction of an individual's responsiveness to an insulin sensitizer, e.g., PPAR modulator is used to include or exclude the individual in a clinical trial. In some embodiments, the prediction of an individual's responsiveness to an insulin sensitizer, e.g., PPAR modulator is used to modulate the individual's administration of another insulin sensitizer, e.g., PPAR modulator. In some embodiments such modulation occurs in a clinical trial. In some embodiments, the prediction of an individual's responsiveness to an insulin sensitizer, e.g., PPAR modulator is used to determine that the individual should be treated with a drug other than an insulin sensitizer, or in some embodiments a PPAR modulator.

In one some embodiments two or more different drugs can be delivered using the DL-NAMs of the invention, for example, where the co-administration of the drugs produces a synergistic effect, enhanced therapeutic effect or other desired outcome.

Mechanistic Classes of Drugs

One non-exclusive exemplary classes of drugs for which genotyping and association studies with one member may be used to predict effects of another member include, mechanistic classes of drugs used in the treatment of diabetes (including PPAR modulators). This class of drugs also illustrates how drugs can also be subclassed by, e.g., mode of administration. For example, insulin and insulin analogs may be formulated for administration by injection, nasal spray, transdermal, oral or inhalation routes. Each type of formulation can have unique profiles of responses and associated genetic variations. An example of classifications of such drugs by mechanism, together with representative members of the mechanistic classes, is given in the table below.

TABLE 6
Classes of Drugs for Treatment of Diabetes

| Class | Mechanism of Action | Examples |
|---|---|---|
| Peroxisome | Target PPAR-gamma or PPAR-gamma and -alpha (see below). | Rosiglitazone, Pioglitazone, |
| Proliferator- | PPAR are nuclear receptors that help regulate glucose and lipid | Balaglitazone, see also |
| Activated Receptor | metabolism. Activation of PPAR-gamma improves insulin | others described herein |
| (PPAR) Agonists | sensitivity and thus improves glycemic control. | |
| Dual-Action | Act on both PPAR-gamma and PPAR-alpha. PPAR-alpha | TAK-559, Muraglitazar, |
| Peroxisome | activation has effects on cellular uptake of fatty acids and their | Tesaglitazar, Netoglitazone, |
| Proliferator- | oxidation, and on lipoprotein metabolism. May also act to reduce | see also others described |
| Activated Receptor | inflammatory response in vascular endothelial cells. | herein |
| Agonists | | |
| Biguanidines | Complete mechanism is not known. Reduces gluconeogenesis in | Metformin, Metformin GR |
| | the liver by inhibiting glucose-6-phosphatase. | |
| Sulfonylureas | Induce insulin secretion by binding to cellular receptors that | Glimepride, |
| | cause membrane depolarization and insulin exocytosis. | Glyburide/glibenclamide, |
| | | Glipizide, Gliclazide. |
| | | Tobutamide |
| Insulin and Insulin | Supplements endogenous insulin. Insulin analogs have a variety | Insulin lispro, Insulin aspart, |
| Analogs (Injectable, | of amino acid changes and have altered onset of action and | Insulin glargine, Exubera, |
| Inhaled, Oral, | duration of action, as well as other properties, compared to native | AERx Insulin Diabetes |
| Transdermal, | insulin. Inhaled insulin is absorbed through the alveoli. Spray | Management System, HIM- |
| Intranasal) | oral insulin is absorbed by the buccal mucosa and intranasal | 2, Oaralin, Insulin detemir, |
| | through the nasal mucosa. Transdermal insulin is absorbed | Insulin glulisine |
| | through the skin. | |
| Meglitinides | Are thought to bind to a nonsulfonylurea beta cell receptor and | Repaglinide, Nateglinide, |
| | act to cause insulin secretion by mechanism similar to | Mitiglinide |
| | sulfonylureas | |
| Alpha-Glucosidase | Inhibit carbohydrate digestion. Act at brush border of intestinal | Acarbose, Miglitol, |
| Inhibitors | epithelium. | Voglibose |
| Glucagon-Like | Diabetic patients may lack native GLP-1, and anlalogs act as | Exenatide, Exenatide LAR, |
| Peptide(GLP)-1 | substitutes. GLP-1 is an intestinal peptide hormone that induces | Liraglutide, ZP 10, |
| Analogs | glucose-dependent insulin secretion, controls gastric emptying, | BN51077, |
| | inhibits appetite, and modulates secretion of glucagon and | |
| | somatostatin. | |
| Dipeptidyl Peptidase | Inhibit DPP-IV, a ubiquitous enzyme that cleaves and inactivates | LAF-237, p-32/98, MK- |
| (DPP)-IV Inhibitors | GLP-1, thus inhibition of DPP-IV increases GLP-1 activity | 431, P3298, NVP LAF 237, |
| Pancreatic Lipase | Inhibits lipases, thus inhibiting uptake of dietary fat. This causes | Orlistat |
| Inhibitors | weight loss, improves insulin sensitivity and lowers | |
| | hyperglycemia. | |
| Amylin Analogs | Act to augment amylin, which acts with insulin by slowing | Pramlintide |
| | glucose absorption from the gut and slows after-meal glucose | |
| | release from liver. | |
| Dopamine D2 | Thought to act to alleviate abnormal daily variations in central | Bromocriptine |
| receptor agonists | neuroendocrine activity that can contribute to metabolic and | |
| | immune system disordered. | |
| Immunosuppressants | Suppress autoimmune response thought to be implicated in | Daclizumab, NBI 6024, |
| | Type I and possibly Type II diabetes. Example: Humanized | TRX-TolerRx, OKT3- |
| | monoclonal antibody that recognizes and inhibits the alpha | gamma-1-ala-ala |
| | subunit of IL-2 receptors; humanized Mab that binds to T cell | |
| | CD3 receptor to block function of T-effector cells that attack the | |
| | body and cause autoimmune disease | |
| Insulin-like growth | Recombinant protein complex of insulin-like growth factor-1 and | Somatomedin-1 binding |
| factor-1 agonists | binding protein-3; regulates the delivery of somatomedin to target | protein 3 |
| | tissues. Reduces insulitis severity and beta cell destruction | |
| Insulin sensitizers | Insulin sensitizers, generally orally active | S15261, Dexlipotam, CLX |
| | | 0901, R 483, TAK 654 |
| Growth hormone | Mimic the action of native GHRF | TH9507, SOM 230 |
| releasing factor | | |
| agonists | | |
| Glucagon antagonists | Inhibit glucagon action, stimulating insulin production and | Liraglutide, NN 2501 |
| | secretion, resulting in lower postprandial glucose levels | |
| Diabetes type 1 | Prevents destruction of pancreatic beta cells that occurs in type 1 | Q-Vax, Damyd vaccine |
| vaccine | diabetes | |
| Sodium-glucose co- | Selectively inhibits the sodium glucose co-transporter, which | T 1095 |
| transporter inhibitor | mediates renal reabsorption and intestinal absorption of glucose | |
| | to maintain appropriate blood glucose levels. | |
| Glycogen | Inhibit glycogen phosphorylase, thus slowing release of glucose | Ingliforib |
| phosphorylase | | |
| inhibitors | | |
| Undefined | Drugs that act in ways beneficial to those with Type I or Type II | FK 614, INGAP Peptide, R |
| mechanisms | Diabetes Mellitus, e.g., by reducing blood glucose and | 1439 |
| | triglyceride levels, whose mechanisms have not been elucidated. | |
| Antisense | Bind to RNA and cause its destruction, thereby decreasing | ISIS 113715 |
| oligonucleotides | protein production from corresponding gene. | |
| Insulinotropin | Stimulate insulin release | CJC 1131 |
| agonists | | |
| Gluconeogenesis | Inhibit gluconeogenesis, thus modulating blood glucose levels | CS 917 |
| inhibitors | | |
| Hydroxysteroid | Inhibit hydroxysteroid dehydrogenase, which are responsible for | BVT 3498 |
| dehydrogenase | excess glucocorticoid production and hence, visceral obesity | |
| inhibitors | | |
| Beta 3 adrenoceptor | Agonist for beta 3 adrenoceptor, decreases blood glucose and | YM 178, Solabegron, |
| agonist | suppresses weight gain | N5984, |
| Nitric oxide | Decreases effects of NO | NOX 700 |
| antagonist | | |
| Carnitine | Inhibits carnitine palmitoyltransferase | ST 1326 |
| palmitoyltransferase | | |
| inhibitor | | |

In other embodiments, mechanistic classes of drugs used in the treatment of abnormal cholesterol and/or triglyceride levels in the blood are used in conjunction with a method or composition of the invention. Broad mechanistic classes include the statins, fibrates, cholesterol absorption inhibitors, nicotinic acid derivatives, bile acid sequestrants, cholesteryl ester transfer protein inhibitors, reverse lipid transport pathway activators, antioxidants/vascular protectants, acyl-CoA cholesterol acyltransferase inhibitors, peroxisome proliferator activated receptor agonists, microsomal triglyceride protein inhibitors, squalene synthase inhibitors, lipoprotein lipase activators, lipoprotein (a) antagonists, and bile acid reabsorption inhibitors. An example of classification of such drugs by mechanism, together with representative members of the mechanistic classes, is given in the Table below.

TABLE 7
Classes of Drugs for Treatment of Abnormal Cholesterol and/or Triglyceride Levels in the Blood

| Class | Mechanism of Action | Examples |
|---|---|---|
| Statins | Competitive inhibitors of HMG-CoA reductase | Atorvastatin, Simvastatin, |
| | | Pravastatin, Fluvastatin, |
| | | Rosuvastatin, Lovastatin, |
| | | Pitavastatin, Cerivastatin |
| | | (withdrawn), |
| Fibrates | PPARα activators | Fenofibrate, Bezafibrate, |
| | | Gemfibrozil, clofibrate, |
| | | ciprofibrate |
| Cholesterol | May inhibit NCP1L1 in gut | Ezetimibe |
| Absorption Inhibitors | | |
| Nicotinic Acid | Inhibits cholesterol and triglyceride synthesis, exact mechanism | Niacin |
| Derivatives | unknown | |
| Bile Acid | Interrupt the enterohepatic circulation of bile acids | Colesevelam, |
| Sequestrants | | Cholestyramine, |
| | | Colestimide, Colestipol |
| Cholesteryl Ester | Inhibit cholesteryl ester transfer protein, a plasma protein that | JTT-705, CETi-1, |
| Transfer Protein | mediates the exchange of cholesteryl esters from antiatherogenic | Torcetrapib |
| Inhibitors | HDL to proatherogenic apoliprotein B-containing lipoproteins | |
| Reverse Lipid | Stimulate reverse lipid transport, a four-step process form | ETC-216, ETC-588, ETC- |
| Transport Pathway | removing excess cholesterol and other lipids from the walls of | 642, ETC-1001, ESP-1552, |
| Activators | arteries and other tissues | ESP-24232 |
| Antioxidants/Vascular | Inhibit vascular inflammation and reduce cholesterol levels; | AGI-1067, Probucol |
| Protectants | block oxidant signals that switch on vascular cellular adhesion | (withdrawn) |
| | molecule (VCAM)-1 | |
| Acyl-CoA | Inhibit ACAT, which catalyzes cholesterol esterification, | Eflucimibe, Pactimibe, |
| Cholesterol | regulates intracellular free cholesterol, and promotes cholesterol | Avasimibe (withdrawn), |
| Acyltransferase | absorption and assemble of VLDL | SMP-797 |
| (ACAT) Inhibitors | | |
| Peroxisome | Activate PPARs, e.g., PPARα, γ, and possibly δ, which have a | Tesaglitazar, GW-50516, |
| Proliferator Activated | variety of gene regulatory functions | GW-590735, LY-929, LY- |
| Receptor Agonists | | 518674, LY-465608, LY- |
| | | 818 |
| Microsomal | Inhibit MTTP, which catalyze the transport of triglycerides, | Implitapide, CP-346086 |
| Triglyceride Transfer | cholesteryl ester, and phosphatidylcholine between membranes; | |
| Protein (MTTP) | required for the synthesis of ApoB. | |
| Inhibitors | | |
| Squalene Synthase | Interfere with cholesterol synthesis by halting the action of liver | TAK-475, ER-119884 |
| Inhibitors | enzymes; may also slow or stop the proliferation of several cell | |
| | types that contribute to atherosclerotic plaque formation | |
| Lipoprotein Lipase | Directly activate lipoprotein lipase, which promotes the | Ibrolipim (NO-1886) |
| Activators | breakdown of the fat portion of lipoproteins | |
| Liproprotein(a) | Not yet established | Gembacene |
| Antagonists | | |
| Bile Acid | Inhibit intestinal epithelial uptake of bile acids. | AZD-7806, BARI-1453, S- |
| Reabsorption | | 8921 |
| Inhibitors | | |

In other embodiments, mechanistic classes of drugs used in the treatment of depression are used in conjunction with a method or composition of the invention. Current or emerging antidepressant drugs act by a variety of mechanisms, e.g., selective serotonin reuptake inhibitors (SSRIs), serotonergic/noradrenergic agents, serotonin/noradrenergic/dopaminergic agents, tricyclic antidepressants, monoamine oxidase inhibitors (MAOIs), noradrenergic/dopaminergic agents, serotonin antagonists, serotonin agonists, substance P antagonists, and beta3 adrenoreceptor agonists. An example of classification of such drugs by mechanism, together with representative members of the mechanistic classes, is given in the Table below.

TABLE 8
Classes of Drugs for Treatment of depression

| Class | Mechanism of Action | Examples |
|---|---|---|
| Selective Serotonin | Block presynaptic reuptake of serotonin. Exert little effect on | Escitalopram, Sertraline, |
| Reuptake Inhibitor | norepinephrine or dopamine reuptake. Level of serotonin in | Citalopram, Paroxetine, |
| (SSRI) | the synaptic cleft is increased. | Paroxetin, controlled |
| | | release, Fluoxetine, |
| | | Fluoxetine weekly, |
| | | Fluvoxamine, |
| | | olanzapine/fluoxetine |
| | | combination |
| Serotonergic/noradrenergic | Inhibit both serotonin reuptake and norepinephrine reuptake. | Venlafaxine; Reboxetine, |
| agents | Different drugs in this class can inhibit each receptor to | Milnacipran, Mirtazapine, |
| | different degrees. Do not affect histamine, acetylcholine, and | Nefazodone, Duloxetine |
| | adrenergic receptors. | |
| Serotonergic/noradrenergic/ | Several different mechanisms. Block norepinephrine, | Bupropion, Maprotiline, |
| dopaminergic agents | serotonin, and/or dopamine reuptake. Some have addictive | Mianserin, Trazodone, |
| | potential due to dopamine reuptake inhibition. | Dexmethylphenidate, |
| | | Methyphenidate, |
| | | Amineptine |
| Tricyclic Antidepressants | Block synaptic reuptake of serotonin and norepinephrine. | Amitriptyline, Amoxapine, |
| | Have little effect on dopamine. Strong blockers of | Clomipramine, |
| | muscarinic, histaminergic H1, and alpha-1-adrenergic | Desipramine, Doxepin, |
| | receptors. | Imipramine, Nortriptyline, |
| | | Protriptyline, Trimipramine |
| Irreversible Monoamine | Monoamine oxidase (MAO) metabolizes monoamines such as | Isocarboxazid, Phenelzine, |
| Oxidase Inhibitors | serotonin and norepinephrine. MAO inhibitors inhibit MAO, | Tranylcypromine, |
| | thus increasing levels of serotonin and norepinephrine. | Transdermal Selegiline |
| Reversible Monoamine | See above. Short acting, reversible inhibitor, inhibits | Moclobemide |
| Oxidase Inhibitors | deamination of serotonin, norepinephrine, and dopamine. | |
| Serotonergic/noradrenergic/ | Act to block all of serotonin, norepinephrine, and dopamine | DOV-216303, DOV-21947 |
| dopaminergic reuptake | reuptake. May have addictive potential due to dopamine | |
| inhibitors | reuptake inhibition. | |
| Noradrenergic/dopaminergic | Block reuptake of norepinephrine and dopamine | GW-353162 |
| agents | | |
| Serotonin Antagonists | Selective antagonist of one serotonin receptor (the 5-HT₁ | Agomelatine |
| | receptor) | |
| Serotonin Agonists | Partial agonist of the 5-HT_{1A} receptor. | Eptapirone, Vilazodone, |
| | | OPC-14523, MKC-242, |
| | | Gepirone ER |
| Substance P Antagonists | Modify levels of substance P, which is released during acute | Aprepitant, TAK-637, CP- |
| | stress. | 122721, E6006, R-763OPC- |
| | | GW-597599 |
| Beta₃ Adrenoreceptor | Indirectly inhibit norepinephrine reuptake. Also being | SR-58611 |
| Agonists | investigated for treatment of obesity and diabetes because | |
| | they stimulate lipolysis and thermogenesis. | |

In other embodiments, mechanistic classes of drugs used in the treatment of multiple sclerosis are used in conjunction with a method or composition of the invention. These drugs can be classed as, e.g., recombinant interferons, altered peptide ligands, chemotherapeutic agents, immunosuppressants, corticosteroids, monoclonal antibodies, chemokine receptor antagonists, AMPA receptor antagonists, recombinant human glial growth factors, T-cell receptor vaccines, and oral immunomodulators. An example of classification of such drugs by mechanism, together with representative members of the mechanistic classes, is given in the Table below.

TABLE 9
Classes of Drugs for Treatment of Multiple Sclerosis

| Class | Mechanism of Action | Examples |
|---|---|---|
| Recombinant | IFN-beta has numerous effects on the immune system. Exact | Interferon-beta-1b, |
| interferons | mechanism of action in MS not known | Interferon-beta-1a |
| Altered peptide | Ligands either templated on sequence of myelin basic protein, or | Glatiramer acetate, MBP- |
| ligands | containing randomly arranged amino acids (e.g., ala, lys, glu, tyr) | 8298, Tiplimotide, AG-284 |
| | whose structure resembles myelin basic protein, which is thought | |
| | to be an antigen that plays a role in MS. Bind to the T-cell | |
| | receptor but do not activate the T-cell because are not presented | |
| | by an antigen-presenting cell. | |
| Chemotherapeutic | Immunosuppressive. MS is thought to be an autoimmune | Mitoxantrone, |
| agents | disease, so chemotherapeutics that suppress immunity improve | Methotrexate, |
| | MS | Cyclophosphamide |
| Immunosuppressants | Act via a variety of mechanisms to dampen immune response. | Azathioprine, |
| | | Teriflunomide, Oral |
| | | Cladribine |
| Corticosteroids | Induce T-cell death and may up-regulate expression of adhesion | Methylprednisolone |
| | molecules in endothelial cells lining the walls of cerebral vessels, | |
| | as well as decreasing CNS inflammation. | |
| Monoclonal | Bind to specific targets in the autoimmune cascade that produces | Natalizumab, Daclizumab, |
| Antibodies | MS, e.g., bind to activated T-cells | Altemtuzumab, BMS- |
| | | 188667, E-6040, Rituximab, |
| | | M1 MAbs, ABT 874, T- |
| | | 0047 |
| Chemokine Receptor | Prevent chemokines from binding to specific chemokine | BX-471, MLN-3897, MLN- |
| Antagonists | receptors involved in the attraction of immune cells into the CNS | 1202 |
| | of multiple sclerosis patients, and inhibiting immune cell | |
| | migration into the CNS | |
| AMPA Receptor | AMPA receptors bind glutamate, an excitatory neurotransmitter, | E-2007 |
| Antagonists | which is released in excessive quantities in MS. AMPA | |
| | antagonists suppresses the damage caused by the glutamate | |
| Recombinant Human | GGF is associated with the promotion and survival of | Recombinant Human GGF2 |
| Glial Growth Factor | oligodendrocytes, which myelinate neurons of the CNS. rhGGF | |
| (GGF) | may help myelinate oligodendrocytes and protect the myelin | |
| | sheath. | |
| T-cell Receptor | Mimic the part of the receptor in T cells that attack myelin | NeuroVax |
| Vaccine | sheath, which activates regulatory T cells to decrease pathogenic | |
| | T-cells. | |
| Oral | Various effects on the immune response that can modulate the | Simvastatin, FTY-720, Oral |
| Immunomodulators | process of MS | Glatiramer Acetate, FTY- |
| | | 720, Pirfenidone, |
| | | Laquinimod |

In other embodiments, mechanistic classes of drugs used in the treatment of Parkinson's disease are used in conjunction with a method or composition of the invention. These classes include dopamine precursors, dopamine agonists, COMT inhibitors, MAO-B inhibitors, antiglutametergic agents, anticholinergic agents, mixed dopaminergic agents, adenosine A2a antagonists, alpha-2 adrenergic antagonists, antiapoptotic agents, growth factor stimulators, and cell replacements. An example of classification of such drugs by mechanism, together with representative members of the mechanistic classes, is given the Table below.

TABLE 10
Classes of Drugs for Treatment of Parkinson's Disease

| Class | Mechanism of Action | Examples |
|---|---|---|
| Dopamine Precursors | Act as precursors in the synthesis of dopamine, the | Levodopa, Levodopa- |
| | neurotransmitter that is depleted in Parkinson's Disease. Usually | carbidopa, Levodopa- |
| | administered in combination with an inhibitor of the carboxylase | benserazide, Etilevodopa, |
| | enzyme that metabolizes levodopa. Some (e.g., Duodopa) are | Duodopa |
| | given by infusion, e.g., intraduodenal infusion | |
| Dopamine Agonists | Mimic natural dopamine by directly stimulating striatal dopamine | Bromocriptine, Cabergoline, |
| | receptors. May be subclassed by which of the five known | Lisuride, Pergolide, |
| | dopamine receptor subtypes the drug activates; generally most | Pramipexole, Ropinirole, |
| | effective are those that activate receptors the in the D2 receptor | Talipexole, Apomorphine, |
| | family (specifically D2 and D3 receptors). Some are formulated | Dihydroergocryptine, |
| | for more controlled release or transdermal delivery. | Lisuride, Piribedil, |
| | | Talipexole, Rotigotin CDS, |
| | | Sumanirole, SLV-308 |
| COMT Inhibitors | Inhibits COMT, the second major enzyme that metabolized | Entacapone, Tolcapone, |
| | levodopa. | Entacapone-Levodopa- |
| | | Carbidopa fixed |
| | | combination, |
| MAO-B Inhibitors | MAO-B metabolizes dopamine, and inhibitors of MAO-B thus | Selegiline, Rasagiline, |
| | prolong dopamine's half-life | Safinamide |
| Antiglutamatergic | Block glutamate release. Reduce levodopa-induced dyskinesia | Amantadine, Budipine, |
| Agents | | Talampanel, Zonisamide |
| Anticholinergic | Thought to inhibit excessive cholinergic activity that | Trihexyphenidyl, |
| Agents | accompanies dopamine deficiency | Benztropine, Biperiden |
| Mixed Dopaminergic | Act on several neurotransmitter systems, both dopaminergic and | NS-2330, Sarizotan |
| Agents | nondopaminergic. | |
| Adenosine A2a | Adenosine A2 antagonize dopamine receptors and are found in | Istradefylline |
| antagonists | conjunction with dopamine receptors. Antagonists of these | |
| | receptors may enhance the activity of dopamine receptors. | |
| Alpha-2 Adrenergic | Not known. | Yohimbine, Idazoxan, |
| Antagonists | | Fipamezole |
| Antiapoptotic Agents | Can slow the death of cells associated with the neurodegenerative | CEP-1347, TCH-346 |
| | process of Parkinson's disease. | |
| Growth Factor | Promote the survival and growth of dopaminergic cells. | GPI-1485, Glial-cell-line- |
| Stimulators | | derived Neurotrophic |
| | | Factor, SR-57667, PYM- |
| | | 50028 |
| Cell Replacement | Replace damaged neurons with health neurons. | Spheramine |
| Therapy | | |

The above classifications are exemplary only. It will be appreciated that a drug class need not be restricted to drugs used in the treatment of a single disease, but that a given mechanistic class may have members useful in the treatment of a number of diseases. For example, MAO-B inhibitors are useful in the treatment of both Parkinson's disease and depression; as another example, statins are useful in the treatment of dyslipidemias but are also being found to have more general use in diseases where inflammation plays a major role, e.g., multiple sclerosis and other diseases.

Further classifications of drugs by mechanism are known in the art; often these classifications may be further classified by structure. Non-exclusive examples of drug classes useful in the methods and compositions of the invention, and representative members of these classes, include:

Sedative-Hypnotic Drugs, which include drugs that bind to the GABAA receptor such as the benzodiazepines (including alprazolam, chlordiazepoxide, clorazepate, clonazepam, diazepam, estazolam, flurazepam, halazepam, lorazepam, midazolam, oxazepam, quazepam, temazepam, triazolam), the barbiturates (such as amobarbital, pentobarbital, phenobarbital, secobarbita), and non-benzodiazepines (such as zolpidem and zaleplon), as well as the benzodiazepine antagonists (such as flumazenil). Other sedative-hypnotic drugs appear to work through non-GABA-ergic mechanisms such as through interaction with serotonin and dopaminergic receptors, and include buspirone, isapirone, geprirone, and tandospirone. Older drugs work through mechanisms that are not clearly elucidated, and include chloral hydrate, ethchlorvynol, meprobamate, and paraldehyde.

In some embodiments, sedative-hypnotic drugs that interact with the GABA receptor, such as benzodiazepines and non-benzodiazepines, are further classified as to which subunit or subunits of the GABAA receptor that they interact with, e.g., the a (which is further classified into six subtypes, including a-1,2,3, and 5), 13 (further classified as four different types), γ (three different types), δ, ε, π, ρ, etc. Such a classification can allow further refinement of associations between genetic variation and responsiveness to a given sedative-hypnotic that interacts with a particular subclass, and predictions for a new sedative-hypnotic that interacts with the same subclass of receptors.

Opioid analgesics and antagonists act on the opioid receptor. The majority of currently available opioid analgesics act primarily at the μ opioid receptor. However, interactions also occur with the δ and κ receptors. Similar to the sedative-hypnotics, in some embodiments opioid analgesics are further classed as to subtypes of receptors at which they primarily interact, thus allowing further refinement of the association between drug response and genetic variation, and higher predictability for a new drug, based on which receptor(s) it interacts with. Opioid analgesics include alfentanil, buprenorphine, butorphanol, codeine, dezocine, fentanyl, hydromorphone, levomethadyl acetate, levorphanol, meperidine, methadone, morphine sulfate, nalbuphine, oxycodone, oxymorphone, pentazocine, propoxyphene, remifentanil, sufentanil, tramadol; analgesic combinations such as codeine/acetaminophen, codeine/aspirin, hydrocodone/acetaminophen, hydrocodone/ibuprofen, oxycodone/acetaminophen, oxycodone/aspirin, propoxyphene/aspirin or acetaminophen. Opioid antagonists include nalmefene, naloxone, naltrexone. Antitussives include codeine, dextromethorphan.

Nonsteroidal anti-inflammatory drugs act primarily through inhibition of the synthesis of prostaglandins, e.g., through inhibition of COX-1, COX-2, or both. Older NSAIDS (e.g., salicylates) tend to be non-selective as to the type of COX inhibited, whereas newer drugs are quite selective (e.g., the COX-2 inhibitors). Non-selective COX inhibitors include aspirin, acetylsalicylic acid, choline salicylate, diclofenac, etodolac, fenoprofen, flurbiprofen, ibuprofen, indomethacin, ketoprofen, ketorolac, magnesium salicylate, meclofenamate, mefenamic acid, nabumetone, naproxen, oxaprozin, phenylbutazone, piroxicam, salsalate, salicylsalicylic acid, sodium salicylate, sodium thiosalicylate, sulindac, tenoxicam, tiaproven, azapropazone, carprofen, and tolmetin. Selective COX-2 inhibitors include celecoxib, etroricoxib, meloxicam, rofecoxib, and valdecoxib.

Histamine agonists and antagonists are classified according to receptor subtype. H1 agonists or partial agonists include 2-(m-fluorophenyl)-histamine and antagonists include chlorpheniramine, scopolamine, mepyramine, terfenadine, astemizole, and triprolidine; further antagonists (which may be further classified by their chemical structures) include the ethanolamines carbinoxamine, dimenhydrinate, diphenhydramine, and doxylamine; the ethylaminediamines pyrilamine and tripelennamine; the piperazine derivatives dydroxyzine, cyclizine, fexofenadine and meclizine; the alkylamines brompheniramine and chlorpheniramine; and miscellaneous antagonists cyproheptadine, loratadine, cetrizine. H2 agonists include dimaprit, impromidine, and amthamine; and antagonists (useful in the treatment of gastric acid secretion) include cimetidine, ranitidine, nizatidine, and famotidine; H3 agonists include R-alpha-methylhistamine, imetit, and immepip and antagonists include thioperamide, iodophenpropit, and clobenpropit; and H4 agonists include clobenpropit, imetit, and clozapine and antagonists include thioperamide. Available preparations include the H1 blockers azelastine, brompheniramine, buclizine, carbinoxamine, cetrizine, chlorpheniramine, clemastine, cyclizine, cyproheptadine, desloratidine, dimenhydrinate, diphenhydramine, emedastine, fexofenadine, hydroxyzine, ketotifen, levocabastine, loratadine, meclizine, olopatadine, phenindamine, and promoathazine.

Drugs used in asthma include sympatheticomimetics (used as “relievers,” or bronchodilators) such as albuterol, albuterol/lpratropium, bitolterol, ephedrine, epinephrine, formoterol, isoetharine, isoproterenol, levalbuterol, metaproterenol, pirbuterol, salmeterol, salmeterol/fluticasone, terbutaline; aerosol corticosteroids (used as “controllers,” or antiinflammatory agents) such as beclomethasone, budesonide, flunisolide, fluticasone, fluticasone/salmeterol, triamcinolone; leukotriene inhibitors such as montelukast, zafirlukast, zileuton; cormolyn sodium and nedocromil sodium; methylxanthines such as aminophylline, theophyllinem dyphylline, oxtriphylline, pentoxifylline; antimuscarinic drugs such as ipratropium; and antibodies such as omalizumab.

Erectile dysfunction drugs include cGMP enhancers such as sildenafil (Viagra), tadalafil, vardenafil, and alprostadil, and dopamine releasers such as apomorphine

Drugs used in the treatment of gastrointestinal disease act by a number of mechanisms. Drugs that counteract acidity (antacids) include aluminum hydroxide gel, calcium carbonate, combination aluminum hydroxide and magnesium hydroxide preparation. Drugs that act as proton pump inhibitors include esomeprazole, lansoprazole, pantoprazole, and rabeprazole. H2 histamine blockers include cimetidine, famotidine, nizatidine, ranitidine. Anticholinergic drugs include atropine, belladonna alkaloids tincture, dicyclomine, glycopyrrolate, I hyoscyamine, methscopolamine, propantheline, scopolamine, tridihexethyl. Mucosal protective agents include misoprostol, sucralfate. Digestive enzymes include pancrelipase. Drugs for motility disorders and antiemetics include alosetron, cisapride, dolasetron, dronabinol, granisetron, metoclopramide, ondansetron, prochlorperazine, tegaserod. Antiinflammatory drugs used in gastrointestinal disease include balsalazide, budesonide, hydrocortisone, mesalamine, methylprednisone, olsalazine, sulfasalazine, infliximab. Antidiarrheal drugs include bismuth subsalicylate, difenoxin, diphenoxylate, kaolin/pectin, loperamide. Laxative drugs include bisacodyl, cascara sagrada, castor oil, docusate, glycerin liquid, lactulose, magnesium hydroxide [milk of magnesia, Epson Salt], methylcellulose, mineral oil, polycarbophpil, polyethylene glycol electrolyte solution, psyllium, sienna. Drugs that dissolve gallstones include monoctanoin, ursodiol.

Cholinoceptor-activating drugs, which act by activating muscarinic and/or nicotinic receptors include esters of choline (e.g., acetylcholine, metacholine, carbamic acid, carbachol, and bethanechol) and alkaloids (e.g., muscarine, pilocarpine, lobeline, and nicotine); cholinesterase-inhibiting drugs which typically act on the active site of cholinesterase include alcohols bearing a quaternary ammonium group (e.g., edrophonium), carbamates and related agents (e.g., neostigmine, physostigmine, pyridostigmine, ambenonium, and demercarium), and organic derivatives of phosphoric acid (e.g., echothiophate, soman, parthion, malathion); cholinoceptor-blocking drugs typically act as antagonists to nicotinic receptors (further classified as ganglion-blockers, such as hexamethonium, mecmylamine, teteraethylammonium, and trimethaphan; and neuromuscular junction blockers, see skeletal muscle relaxants) or antagonists to muscarinic receptors (e.g. atropine, propantheline, glycopyrrolate, pirenzepine, dicyclomine, tropicamide, ipatropium, banztropine, gallamine, methooctramine, AF-DX 116, telenzipine, trihexyphenidyl, darifenacin, scopolamine, homatropine, cyclopentolate, anisotropine, clidinium, isopropamide, mepenzolate, methscopolamine, oxyphenonium, propantheline, oxybutynin, oxyphencyclimine, propiverine, tolterodine, tridihexethyl), which can be further subclassed as to which muscarinic receptor is the primary site of the effect, e.g., M1, M2, M3, M4, or M5, allowing greater predictability for an association between a genetic variation and a response for a new drug based on its primary site of effect. Available preparations of antimuscarinic drugs include but are not limited to atropine; beladonna alkaloids, extract, or tincture; clidinium; cyclopentolate; dicyclomine; flavoxate; glycopyrrolate; homatropine; 1-hysocyamine; ipratropium; mepenzolate; methantheline; methscopolamine; oxybtynin; prpantehline; scopolamine; tolterodine; tridihexethyl; tropicamide. Available preparations of ganglion blockers include mecamylamine and trimethaphan. Available cholinesterase regenerators include pralidoxime.

Adrenoceptor-activating drugs and other sympathomimetic drugs may be classified according to the receptor or receptors that they activate, e.g., alpha-one type (including subtypes A, B, D), alpha-two type (including subtypes A, B, and C), beta type (including subtypes 1, 2, and 3), and dopamine type (including subtypes 1, 2, 3, 4, and 5. Exemplary drugs include epinephrine, norepinephrine, phenylephrine, methoxamine, milodrine, ephedrine, xylometazoline, amphetamine, methamphetamine, phenmetrazine, methylphenidate, phenylpropanolamine, methylnorepinephrine, dobutamine, clonidine, BHT920, oxymetazoline, isoproterenol, procaterol, terbutaline, metaproterenol, albuterol, ritodrine, BRL37344, dopamine, fenoldopam, bromocriptine, quinpirol, dexmedetomidine, tyramine, cocaine (dopamine reuptake inhibitor), apraclonidine, brimonidine, ritodrine, terbutaline, and modafinil. Available preparations include amphetamine, apraclonidine, brimonidine, dexmedetomidine, dexmthylphenidate, dextroamphetamine, dipivefrin, dobutamine, dopamine, ephedrine, epinephrine, fenoldopam, hydroxyamphetamine, isoproterenol, mephentermine, metaraminol, methamphetamine, methoxamine, methylphenidate, midodrine, modafinil, naphazoline, norepinephrine, oxymetzoline, pemoine, phendimetrazine, phenylephrine, pseudoephedrine, tetrahydrozoline, and xylometaoline.

Adrenoceptor antagonist drugs may be classified by receptor Type In the same manner as adrenoceptor agonists, and include tolazoline, dibenamine, prazosin, terazosin, doxazosin, phenoxybenzamine, phentolamine, rauwoscine, yohimbine, labetalol, carvedilol, metoprololol, acebutolol, alprenolol, atenolol, betaxolol, celiprolol, esmolol, propanolol, carteolol, penbutolol, pindolol, timolol, butoxamine, ergotamine, dihydroergotamine, tamulosin, alfuzosin, indoramin, urapidil, bisoprolol, nadolol, sotalol, oxpenolol, bopindolol, medroxalol, and bucindolol. Available preparations include: alpha blockers doxazosin, phenoxybenzamine, phentolamine, prazosin, tamsulosin, terazosin, and tolazoline; and beta blockers acebutolol, atenolol, betaxolol, bisoprolol, carteolol, carvedilol, esmolol, labetolol, levobunolol, metiproanolol, nadolol, penbutolol, pinolol, propanolol, sotalol, timolol; and synthesis inhibitor metyrosine.

Antihypertensive agents include drugs that work by a variety of mechanisms and thus overlap with other classifications. Agents can include diuretics such as thiazide diuretics, and potassium sparing diurietcs; drugs that act on the central nervous system such as methyldopa and clonidine; ganglion-blocking drugs, suprea; adrenergic neuron-blocking agents such as gunethidine, gunadrel, bethanidine, debrisoquin, and reserpine; adrenoceptor antagonists such as propanolol, metoprolol, nadolol, carteolol, atenolol, betaxolol, bisoprolol, pindolol, acebutolol, and penbutolol, labetalol, carvedilol, esmolol, pazosin, phentolamine and phenoxybenzamine; vasodilators such as hydralzaine, minoxidil, sodium nitroprusside, diazoxide, fenoldopam, and calcium channel blockers (e.g., verapamil, diltiazem, amlopidine, felopidine, isradipine, nicardipine, nifedipine, and nisoldipine); ACE-inhibitors such as captropril, enalapril, lisinopril, benazepril, fosinopril, moexipril, perindopril, quinapril, ramipril, and trandolapril; angiotensin receptor blocking agents such as losartan, valsartan, candesartan, eprosartan, irbesartan, and telmisartan. Preparations available include: beta adrenoceptor blockers acebutolol, atenolol, betaxolol, bisoprolol, carteolol, carvedilol, exmolol, labetalol, metoprolol, nadolol, penbutolol, pindolol, propanolol, timolol; centrally acting sympathoplegic drugs clonidine, gunabenz, guanfacine, methyldopa; postganglionic sympatheic nerve terminal blockers gunadrel, guanethidine, and reserpine; alpha one selective adrenoceptor blockers doxazosin, prazosin, terazosin; ganglion-blocking agent mecamylamine; vasodilators diazoxide, fenoldopam, hydralazine, minoxidil, nitroprusside; calcium channel blockers amlodipine, diltiazem, felodipine, isradipine, nicardipine, nisoldipine, nifedipine, verapamil; ACE inhibitors benazepril, captopril, enalapril, fosinopril, lisinopril, moexipril, perindopril, quinapril, ramipril, and trandolapril; and angiotensin receptor blockers candesartan, eprosartan, irbeartan, losartan, olmisartan, telmisartan, and valsartan.

Vasodilators used in angina pectoris include nitric oxide releasing drugs such as nitric and nitrous acid esters of polyalcohols such as nitroglycerin, isorbide dinitrate, amyl nitrite, and isosorbide mononitrate; calcium channel blockers such as amlodipine, felodipine, isradipine, nicardipine, nifedipine, nimodipine, nisoldipine, nitrendipine, bepridil, diltiazem, and verapamil; and beta-adrenoceptor-blocking drugs (see above). Available preparations include: nitrates and nitrites amyl nitrite, isosorbide dinitrate, isosorbide mononitrate, nitroglycerin; calcium channel blockers amlodipine bepridil, diltiazem, felodipine, isradipine, nicardipine, nifedipine, nimodipine, nisoldipine, and verapamil; and beta blockers acebutolol, atenolol, betaxolol, bisoprolol, carteolol, carvedilol, esmolol, labetolol, levobunolol, metiproanolol, nadolol, penbutolol, pinolol, propanolol, sotalol, timolol.

Drugs used in heart failure include cardiac glycosides such as digoxin; phosphodiesterase inhibitors such as inmrinone and milrinone; beta adrenoceptor stimulant such as those described; diuretics as discussed below; ACE inhibitors such as those discussed above; drugs that inhibit both ACE and neutral endopeptidase such as omaprtrilat; vasodilators such as synthetic brain natriuretic peptide (nesiritide) and bosentan; beta adrenoceptor blockers such as those described above. Available preparations include: digitalis digoxin; digitalis antibody digoxin immune Fab; sympathomimetics dobutamine and dopamine; ACE inhibitors captopril, enalapril, fosinopril, lisinopril, quinapril, ramipril, and trandolapril; angiotensin receptor blockers candesartan, wprosartan, irbesartan, losartan, olmesartan, telmisartan, and valsartan; beta blockers bisoprolol, carvedilol, and metoprolol.

Cardiac arrhythmia drugs include drugs that act by blocking sodium channels such as quinidine, amiodaron, disoprymide, flecamide, lidocaine, mexiletine, morcizine, procainamide, propafeneone, and tocamide; beta-adrenoceptor-blocking drugs such as propanolol, esmolol, and sotalol; drugs that prolong the effective refractory period by prolonging the action potential such as amiodarone, bretylium, sotalol, dofetilide, and ibutilide; calcium channel blockers such as verapamil, diltizem, and bepridil; and miscellaneous agents such as adenosine, digitalis, magnesium, and potassium. Available preparations include: the sodium channel blockers disopryamide, flecamide, lidocaine, miexiletine, moricizine, procainamide, propafenone, quinidine sulfate, quinidine gluconate, and quinidine polygalacturonate; the beta blockers acebutolol, esmolol, and propranolol; the action potential-prolonging agents amiodarone, bretylium, dofetilide, ibutilide, and sotalol; the calcium channel blockers bepridil, diltiazem, and verapamil; and adenosine and magnesium sulfate.

Diuretic agents include drugs that act as carbonic anhydrase inhibitors such as acetazoloamide, dichlorphenamide, methazolamide; loop diuretics such as furosemide, bumetanide, torsemide, ethacrynic acid, and mercurial diuretics; drugs that inhibit NaCl transport in the distal convoluted tubule and, in some cases, also act as carbonic anhydrase inhibitors, such as bendroflumethiazide, benzthiazide, chlorothiazide, chlorthalidone, hydrochlorothiazide, hydroflumethiazide, indapamide, methyclothiazide, metolazone, polythiazide, quinethazone, and trichlormethazide; potassium-sparing diuretics such as spironolactone, triamterene, eplerenone, and amiloride; osmotic diuretics such as mannitol; antidiuretic hormone agonists such as vasopressin and desmopressin; antidiuretic hormone antagonists such aslithium and demeclocycline. Available preparations include actetazolamide, amiloride, bendroflumethiazide, benzthiazide, brinzolamide, bumetanide, chlorothiazide, chlorthalidone, demeclocycline, dichlorphenamide, dorzolamide, eplerenone, ethacrynic acid, furosemide, hydrochlorothiazide, hydroflumethiazide, indapamide, mannitol, methazolamide, methyclothiazide, metolazone, polythiazide, quinethazone, apironolactone, torsemide, triamterene, and trichlormethiazide.

Serotonin and drugs that affect serotonin include serotonin agonists such as fenfluramine and dexfenfluramine, buspirone, sumatriptan, cisapride, tegaserod; seratonin antagonists p-chlorophenylalanine and p-chloroamphetamine, and reserpine; and the serotonin receptor antagonists phenoxybenzamine, cyproheptadine, ketanserin, ritanserin, and ondansetron; serotonin reuptake inhibitors are described elsewhere herein. Serotonin receptor agonists include almotriptan, eletriptan, frovatriptan, naratriptan, rizatriptan, sumatriptan, and zolmitriptan.

Ergot alkaloids are useful in the treatment of, e.g., migraine headache, and act on a variety of targets, including alpha adrenoceptors, serotonin receptors, and dopamine receptors. They include bromocriptine, cabergoline, pergolide, ergonovine, ergotamine, lysergic acid diethylamide, and methysergide. Available preparations include dihydroergotamine, ergonovine, ergotamine, ergotamine tartrate, and methylergonovine.

Vasoactive Peptides include aprepitant, bosentan.

Eicosanoids include prostaglandins, thomboxanes, and leukotrienes. Eicosanoid modulator drugs include alprostadil, bimatoprost, carboprost tromethamine, dinoprostone, epoprostenol, latanoprost, misoprostol, monteleukast, travaprost, treprostinil, unoprostone, zafirleukast, zileuton. Further eicosanoid modulators are discussed elsewhere herein as nonsteroidal antiinflammatory drugs (NSAIDs)

Drugs for the treatment of acute alcohol withdrawal include diazepam, lorazepam, oxazepam, thiamine; drugs for prevention of alcohol abuse include disulfuram, naltrexone; and drugs for the treatment of acute methanol or ethylene glycol poisoning include ethanol, fomepizole.

Antiseizure drugs include carbamazepine, clonazepam, clorazepate dipotassium, diazepam, ethosuximide, ethotoin, felbamate, fosphenyloin, gabapentin, lamotrigine, levetiracetam, lorazepam, mephenyloin, mephobarbital, oxycarbazepine, pentobarbital sodium, phenobarbital, phenyloin, primidone, tiagabine, topiramate, trimethadione, valproic acid.

General anesthetics include desflurane, dexmedetomidine, diazepam, droperidol, enflurane, etomidate, halothane, isoflurane, ketamine, lorazepam, methohexital, methoxyflurane, midazolam, nitrous oxide, propofol, sevoflurane, thiopental.

Local anesthetics include articaine, benzocaine, bupivacaine, butamben picrate, chloroprocaine, cocaine, dibucaine, dyclonine, levobupivacaine, lidocaine, lidocaine and etidocaine eutectic mixture, mepivacaine, pramoxine, prilocalne, procaine, proparacaine, ropivacaine, tetracaine.

Skeletal muscle relaxants include neuromuscular blocking drugs such as atracurium, cisatracurium, doxacurium, metocurine, mivacurium, pancuronium, pipecuronium, rocuronium, succinylcholine, tubocurarine, vecuronium; muscle relaxants (spasmolytics) such as baclofen, botulinum toxin type A, botulinum toxin type B, carisoprodol, chorphenesin, chlorzoxazone, cyclobenzaprine, dantrolene, diazepam, gabapentin, metaxalone, methocarbamol, orphenadrine, riluzole, and tizanidine.

Antipsychotic agents include aripiprazole, chlorpromazine, clozapine, fluphenazine, fluphenazine esters, haloperidol, haloperidol ester, loxapine, mesoridazine, molindone, olanzapine, perphenazine, pimozide, prochlorperazine, promazine, quetiapine, risperidone, thioridazine, thiothixene, trifluoperazine, triflupromazine, ziprasidone; mood stabilizers include carbamazepine, divalproex, lithium carbonate, and valproic acid.

Agents used in anemias include hematopoietic growth factors such as darbopoetin alfa, deferoxamine, epoetin alfa (erythropoetin, epo), filgrastim (G-CSF), folic acid, iron, oprelvekin (interleukin 11), pegfilgrastim, sargramostim (GM-CSF), vitamin B12.

Disease-modifying antirheumatic drugs include anakinra, adalimumab, auranofin, aurothioglucose, etanercept, gold sodium thiomalate, hydroxychloroquine, infliximab, leflunomide, methotrexate, penicillamine, sulfasalazine. Drugs used in gout include allopurinol, colchicine, probenecid, sulfinpyrazone.

Drugs used in disorders of coagulation include abciximab, alteplase recombinant, aminocaproic acid, anisindione, antihemophilic factor [factor VIII, AHF], anti-inhibitor coagulant complex, antithrombin III, aprotinin, argatroban, bivalirudin, cilostazol, clopidogrel, coagulation factor VIIa recombinant, dalteparin, danaparoid, dipyridamole, enoxaparin, eptifibatide, Factor VIIa, Factor VIII, Factor IX, fondaparinux, heparin sodium, lepirudin, phytonadione [K1], protamine, reteplase, streptokinase, tenecteplase, ticlopidine, tinzaparin, tirofiban, tranexamic acid, urokinase, warfarin.

Hypothalamic and pituitary hormones include bromocriptine, cabergoline, cetrorelix, chorionic gonadotropin [hCG], corticorelin ovine, corticotropin, cosyntropin, desmopressin, follitropin alfa, follitropen beta [FSH], ganirelix, gonadorelin acetate [GnRH], gonadorelin hydrochloride [GnRH], goserelin acetate, histrelin, leuprolide, menotropins [hMG], nafarelin, octreotide, oxytocin, pergolide, protirelin, sermorelin, somatrem, somatropin, thyrotropin alpha, triptorelin, urofollitropin, vasopressin.

Thyroid and antithyroid drugs include the thyroid agents: levothyroxine [T4], liothyronine [T3], liotrix [a 4:1 ratio of T4:T3], thyroid desiccated [USP]; and the antithyroid agents: diatrizoate sodium, iodide, iopanoic acid, ipodate sodium, methimazole, potassium iodide, propylthiouracil [PTU], thyrotropin; recombinant human TSH.

Adrenocorticosteroids and adrenocortical antagonists include the glucocorticoids for oral and parenteral use: betamethasone, betamethasone sodium phosphate, cortisone, dexamethasone, dexamethasone acetate, dexamethasone sodium phosphate, hydrocortisone [cortisol], hydrocortisone acetate, hydrocortisone cypionate, hydrocortisone sodium phosphate, hydrocortisone sodium succinate, methylprednisolone, methylprednisolone acetate, methylprednisolone sodium succinate, prednisolone, prednisolone acetate, prednisolone sodium phosphate, prednisolone tebutate, prednisone, triamcinolone, triamcinolone acetonide, triamcinolone diacetate, triamcinolone hexacetonide. Another class of adrenocorticoids are the mineralocorticoids, e.g., fludrocortisone acetate. The adrenal steroid antagonists include aminoglutethimide, ketoconazole, mitotane.

Gonadal hormones and inhibitors include the estrogens: conjugated estrogens, dienestrol, diethylstilbestrol diphosphate, esterified estrogens, estradiol cypionate in oil, estradiol, estradiol transdermal, estradiol valerate in oil, estrone aqueous suspension, estropipate, ethinyl estradiol; the progestins: hydroxyprogesterone caproate, levonorgestrel, medroxyprogesterone acetate, megestrol acetate, norethindrone acetate, norgestrel, progesterone; the androgens and the anabolic steroids: methyltestosterone, nandrolone decanoate, oxandrolone, oxymetholone, stanozolol, testolactone, testosterone aqueous, testosterone cypionate in oil, testosterone enanthate in oil, testosterone propionate in oil, testosterone transdermal system, testosterone pellets. Drugs may further be classed as antagonists and inhibitors of gonadal hormones: anastrozole, bicalutamide, clomiphene, danazol, dutasteride, exemestane, finasteride, flutamide, fulvestrant, letrozole mifepristone, nilutamide, raloxifene, tamoxifen, and toremifene.

Agents that affect bone mineral homeostasis include Vitamin E, its metabolites and analogs: calcifediol, calcitriol, cholecalciferol [D3], dihydrotachysterol [DHT], doxercalciferol, ergocalciferol [D2], and paricalcitol; calcium: calcium acetate [25% calcium], calcium carbonate [40% calcium], calcium chloride [27% calcium], calcium citrate [21% calcium], calcium glubionate [6.5% calcium]; calcium gluceptate [8% calcium], calcium gluconate [9% calcium], calcium lactate [13% calcium], and tricalcium phosphate [39% calcium]; phosphate and phosphate binders such as phosphate and sevelamer; and other drugs such as alendronate, calcitonin-salmon, etidronate, gallium nitrate, pamidronate, plicamycin, risedronate, sodium fluoride, teriparatide, tiludronate, zoledronic acid.

Beta-lactam antibiotics and other inhibitors of cell wall synthesis include the penicillins, such as amoxicillin, amoxicillin/potassium clavulanate, ampicillin, ampicillin/sulbactam sodium, carbenicillin, dicloxacillin, mezlocillin, nafcillin, oxacillin, penicillin G benzathine, penicillin G procaine, penicillin V, piperacillin, pipercillin and tazobactam sodium, ticarcillin, and ticarcillin/clavulanate potassium; the cephalosporins and other beta-lactam drugs, such as the narrow spectrum (first generation) cephalosporins, e.g., cefadroxil, cefazolin, cephalexin, cephalothin, cephapirin, and cephradine; the second generation (intermediate spectrum) cephalosporins, e.g., cefaclor, cefamandole, cefmetazole, cefonicid, cefotetan, cefoxitin, cefprozil, cefuroxime, and loracarbef; the broad spectrum (third- and fourth-generation cephalosporins, e.g., cefdinir, cefditoren, cefepime, cefixime, cefoperazone, cefotaxime, cefpodoxime proxetil, ceftazidime, ceftibuten, ceftizoxime, and ceftriaxone. Further classes include the carbapenem and monobactam, e.g., aztreonam, ertapenem, imipenem/cilastatin, and meropenem; and other drugs such as cycloserine (seromycin pulvules), fosfomycin, vancomycin.

Other antibiotics include chloramphenicol, the tetracyclines, e.g., demeclocycline, doxycycline, methacycline, minocycline, oxtetracycline, and tetracycline; the macrolides, e.g., azithromycin, clarithromycin, erythromycin; the ketolides, e.g., telithromycin; the lincomycins, e.g., clindamycin; the streptogramins, e.g., quinupristin and dalfopristin; and the oxazolidones, e.g., linezolid.

Aminoglycosides and spectinomycin antibiotics include amikacin, gentamicin, kanamycin, neomycin, netilmicin, paromomycin, spectinomycin, streptomycin, and tobramycin.

Sulfonamides, trimethoprim, and quinolone antibiotics include the general-purpose sulfonamides, e.g., sulfadiazine, sulfamethizole, sulfamethoxazole, sulfanilamide, and sulfisoxazole; the sulfonamides for special aplications, e.g., mafenide, silver sulfadiazine, sulfacetamide sodium. Trimethoprims include trimethoprim, trimethoprim-sulfamethoxazole [co-trimoxazole, TMP-SMZ]; the quinolones and fluoroquinolones include cinoxacin, ciprofloxacin, enoxacin, gatifloxacin, levofloxacin, lomefloxacin, moxifloxacin, nalidixic acid, norfloxacin, ofloxacin, sparfloxacin, and trovafloxacin.

Antimycobacterial drugs include drugs used in tuberculosis, e.g., aminosalicylate sodium, capreomycin, cycloserine, ethambutol, ethionamide, isoniazid, pyrazinamide, rifabutin, rifampin, rifapentine, and streptomycin; and drugs used in leprosy, e.g., clofazimine, dapsone.

Antifungal agents include amphotericin B, butaconazole, butenafine, caspofungin, clotrimazole, econazole, fluconazole, flucytosine, griseofulvin, itraconazole, ketoconazole, miconazole, naftifine, natamycin, nystatin, oxiconazole, sulconazole, terbinafine, terconazole, tioconazole, tolnaftate, and voriconazole.

Antiviral agents include abacavir, acyclovir, adefovir, amantadine, amprenavir, cidofovir, delavirdine, didanosine, efavirenz, enfuvirtide, famciclovir, fomivirsen, foscarnet, ganciclovir, idoxuridine, imiquimod, indinavir, interferon alfa-2a, interferon alpha-2b, interferon-2b, interferon alfa-n3, interferon alfacon-1, lamivudine, lopinavir/ritonavir, nelfinavir, nevirapine, oseltamivir, palivizumab, peginterferon alfa-2a, peginterferon alfa-2b, penciclovir, ribavirin, rimantadine, ritonavir, saquinavir, stavudine, tenofovir, trifluridine, valacyclovir, valgancyclovir, zalcitabine, zanamivir, and zidovudine.

Further antimicrobial agents, disinfectants, antiseptics, and sterilants include the miscellaneous antimicrobial agents, e.g., methenamine hippurate, methenamine mandelate, metronidazole, mupirocin, nitrofurantoin, polymyxin B; and the disinfectants, antiseptics, and sterilants, e.g., benzalkonium, benzoyl peroxide, chlorhexidine gluconate, glutaraldehyde, hexachlorophene, iodine aqueous, iodine tincture, nitrofurazone, oxychlorosene sodium, providone-iodine, sliver nitrate, and thimerosal.

Antiprotozoal drugs include albendazole, atovaquone, atovaquone-proguanil, chloroquine, clindamycin, doxycycline, dehydroemetine, eflornithine, halofantrine, iodoquinol, mefloquine, melarsoprol, metronidazole, nifurtimox, nitazoxanide, paromomycin, pentamidine, primaquine, pyrimethamine, quinidine gluconate, quinine, sodium stibogluconate, sulfadoxine and pyrimethamine, and suramin.

Anthelmintic drugs include albendazole, bithionol, diethylcarbamazine, ivermectin, levamisole, mebendazole, metrifonate, niclosamide, oxamniquine, oxantel pamoate, piperazine, praziquantel, pyrantel pamoate, suramin, thiabendazole.

Immunopharmacological agents include abciximab, adalimumab, alefacept, alemtuzumab, anti-thymocyte globulin, azathioprine, basiliximab, BCG, cyclophosphamide, cyclosporine, daclizumab, etanercept, gemtuzumab, glatiramer, ibritumomab tiuxetan, immune globulin intravenous, infliximab, interferon alfa-2a, interferon alfa 2b, interferon beta-1a, interferon beta-1b, interferon gamma-1b, interleukin-2, IL-2, aldesleukin, leflunomide, levamisole, lymphocyte immune globulin, methylprednisolone sodium succinate, muromonab-CD3 [OKT3], mycophenolate mofetil, pegademase bovine, peginterferon alfa-2a, peginterferon alfa-2b, prednisone, RHo(D) immune globulin micro-dose, rituximab, sirolimus, tacrolimus [FK506], thalidomide, and trastuzumab.

Heavy metal chelators include deferoxamine, dimercaprol, edetate calcium [calcium EDTA], penicillamine, succimer, and unithiol.

### Structural Classes of Drugs

In another example of drug classification embodiments, a drug may be classified according to its structural class or family; certain drugs may fall into more than one structural class or family. Thus, in some embodiments, drugs are classified according to structure. Drugs that have a common action may have different structures, and often one of the best predictors of a drugs likely action is its structure. By way of example only, certain classes of drugs may be further organized by chemical structure classes presented herein. One non-limiting example is antibiotics. Table 9, below, presents non-limiting examples of antibiotics further classified by illustrative chemical structure classes.

TABLE 10
Structural Classes of Antibiotic Drugs

| Structure Class | Examples of Antibiotics within Structure Class |
|---|---|
| Amino Acid Derivatives | Azaserine, Bestatin, Cycloserine, |
| | 6-diazo-5-oxo-L-norleucine |
| Aminoglycosides | Armastatin, Amikacin, Gentamicin, |
| | Hygromicin, Kanamycin, Streptomycin |
| Benzochinoides | Herbimycin |
| Carbapenems | Imipenem, Meropenem |
| Coumarin-glycosides | Novobiocin |
| Fatty Acid Derivatives | Cerulenin |
| Glucosamines | 1-deoxynojirimycin |
| Glycopeptides | Bleomycin, Vancomycin |
| Imidazoles | Metroidazole |
| Penicillins | Benzylpenicillin, Benzathine penicillin, |
| | Amoxycillin, Piperacillin |
| Macrolides | Amphotericin B, Azithromycin, Erythromycin |
| Nucleosides | Cordycepin, Formycin A, Tubercidin |
| Peptides | Cyclosporin A, Echinomycin, Gramicidin |
| Peptidyl Nucleosides | Blasticidine, Nikkomycin |
| Phenicoles | Chloramphenicol, Thiamphenicol |
| Polyethers | Lasalocid A, Salinomycin |
| Quinolones | 8-quinolinol, Cinoxacin, Ofloxacin |
| Steroids | Fusidic Acid |
| Sulphonamides | Sulfamethazine, Sulfadiazine, Trimethoprim |
| Tetracyclins | Oxytetracyclin, Minocycline, Duramycin |

In some embodiments, drugs are classed as optical isomers, where a class is two or more optical isomers, or racemate, of a compound of the same chemical formula. Thus, the invention includes methods and compositions for screening individuals for a genetic variation and/or phenotypic variation that predicts responsiveness to a first drug, and using this association to determine whether or not to modulate the treatment of an individual with a second drug, where the first and second drugs are optical isomers. In some embodiments, the first drug is a racemate and the second drug is a stereoisomer that is a component of the racemate. In some embodiments the first drug is a stereoisomer and the second drug is a racemate that includes the stereoisomer. In some embodiments the first drug is a first stereoisomer and the second drug is a second stereoisomer of a compound.

In some embodiments, drugs are classed as different crystal structures of the same formula. Thus, the invention includes methods and compositions for screening individuals for a genetic variation and/or phenotypic variation that predicts responsiveness to a first drug, and using this association to determine whether or not to modulate the treatment of an individual with a second drug, where the first and second drugs are members of a class of drugs of the same chemical formula but different crystal structures.

In some embodiments, drugs are classed by structural components common to the members of the class. Thus, the invention includes methods and compositions for screening individuals for a genetic variation and/or phenotypic variation that predicts responsiveness to a first drug, and using this association to determine whether or not to modulate the treatment of an individual with a second drug, where the first and second drugs are members of a class of drugs that contain the same structural component. By way of example only, a drug may be structurally classified as an acyclic ureide; acylureide; aldehyde; amino acid analog; aminoalkyl ether (clemastine, doxylamine); aminoglycoside; anthracycline; azalide; azole; barbituate; benzodiazapene; carbamate (e.g., felbamate, meprobamate, emylcamate, phenprobamate); carbapenam; carbohydrate; carboxamide (e.g., carbamazepine, oxcarbazepine); carotenoid (e.g., lutein, zeaxanthin); cephalosporin; cryptophycin; cyclodextrin; diphenylpropylamine; expanded porphyrin (e.g., rubyrins, sapphyrins); fatty acid; glycopeptide; higher alcohol; hydantoins (e.g., phenyloin); hydroxylated anthroquinone; lincosamide; lipid; lipid related compound; macrolide; mustard; nitrofuran; nitroimidazole; non-natural nucleotide; non-natural nucleoside; oligonucleotide; organometallic compound; oxazolidinedione; penicillin; phenothiazine derivative (alimemazine, promethazine); phenylpiperidine; phthalocyanine; piperazine derivative (e.g., cetrizine, meclozine); platinum complex (e.g., cis-platin); polyene; polyketide; polypeptide; porphyrin; prostaglandin (e.g., misoprostol, enprostil); purine; pyrazolone; pyrimidine; pyrrolidine (levetiracetam); quinolone; quinone; retinoid (e.g., isotretinoin, tretinoin); salicylate; sphingolipid; steroid (e.g., prednisone, triamcinolone, hydrocortisone); substituted alkylamine (e.g., talastine, chlorphenamine); substituted ethylene diamine (mepyramine, thonzylamine); succinimide (ethosuximide, phensuximide, mesuximide); sulfa; sulfonamide (sulfathiazole, mafenide); sulfone; taxane; tetracycline (e.g., chlortetracycline, oxytetracline); texaphyrin (e.g., Xcytrin, Antrin); thiazide; thiazolidinedione; tocopherol, tocotrienol, triazine (e.g., lamotrigine); urea; xanthine (theobromine, aminophylline); and zwitterion.

### EXAMPLES

### Example 1

The sequences of the strands, shown in Table 1, were designed according to the standards set by Seeman (Seeman, J Biomol Struct Dyn 8:573-81 (1990), which is hereby incorporated by reference in its entirety) and commercially synthesized (Integrated DNA Technologies, Coralville, Iowa). All oligonucleotides were dissolved in annealing buffer (10 mM Tris, pHS. 0.50 mM NaCl, 1 mM EDTA) with a final concentration of 0.1 mM.

Y-shaped DNA (Y-DNA) were synthesized by mixing equal amounts of three oligonucleotide strands. (see FIG. 1A; also as described above, supra, Y-Shape). All the mixtures were first incubated at 95° C. for 2 min, then quickly cooled to 60° C., and finally slowly cooled to 4° C.

Annealing Program:

| | Control | block |
|---|---|---|
| | Lid | 105° C. |
| (Denaturation) | 95° C. | 2 min |
| (Cooling) | 65° C. | 2 min |
| (Annealing) | 60° C. | 5 min |
| (Annealing) | 60° C. | 0.5 min |
| (number of cycle) | Temperature increment −1° C. | |
| | go to (5) Rep 40 | |
| (Hold) | 4° C. | enter |

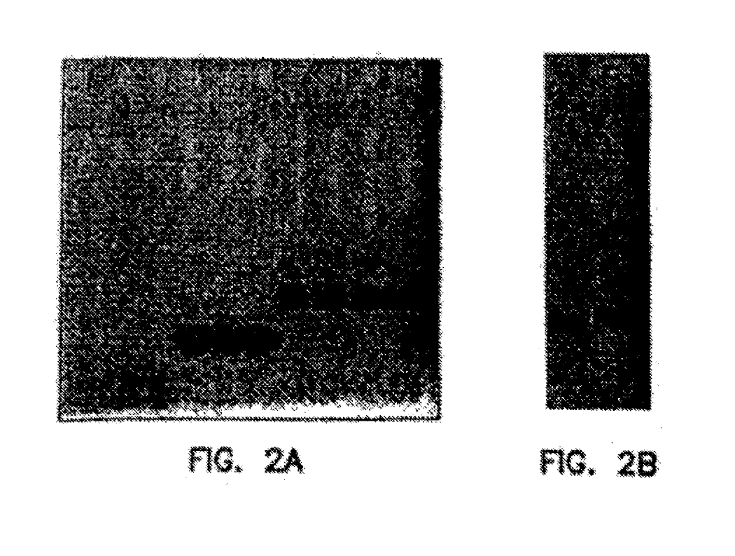

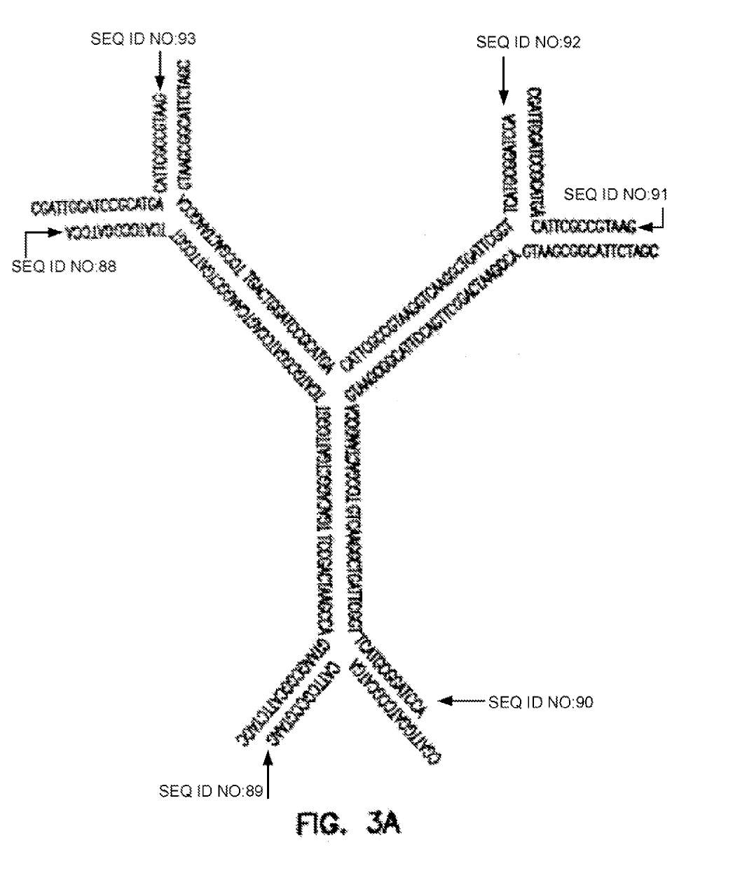

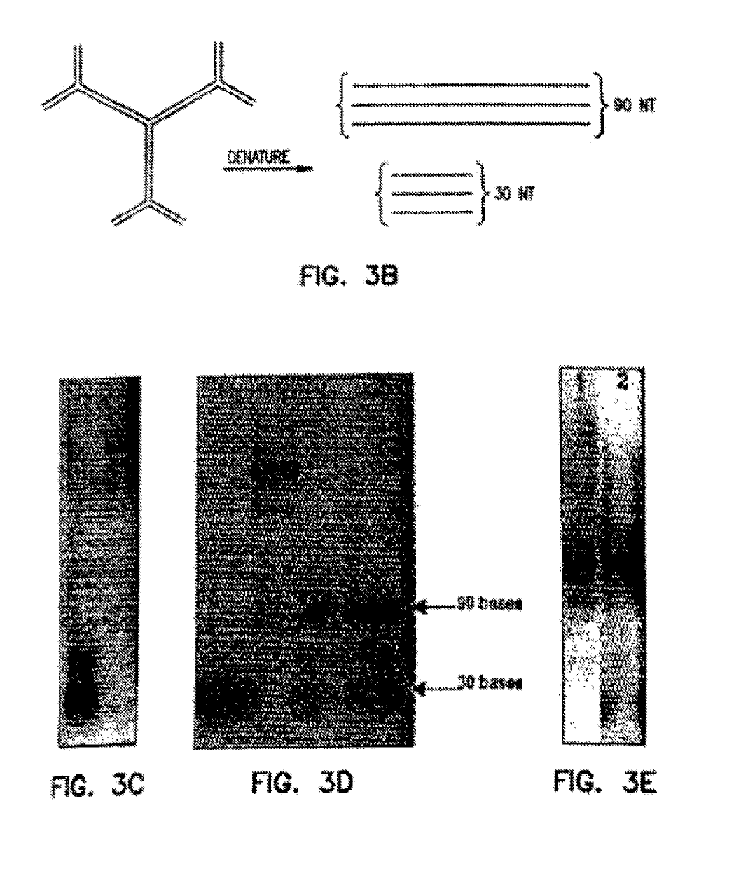

In one embodiment, Y-DNAs are the basic building blocks for the DL-DNMs. Two strategies were adopted to synthesize the Y-DNA: stepwise and all-in-one. In the stepwise approach (FIG. 1A), two oligonucleotides with complementary regions formed one arm of a Y-DNA; then a third oligonucleotide, that was complementary to the first two un-matched regions of oligonucleotides, formed the other two arms of the Y-DNA. In die all-in-one approach, all three oligonucleotides were mixed together in equal amounts to form the Y-DNA. In both cases, high-resolution gel electrophoresis was applied to evaluate, the formation of Y-DNA (FIG. 2).

Nucleic acid samples were evaluated on either 3% agarose ready gel (Bio-Rad, Hercules, Calif.) or 4-20% TBE polyacrylaraide ready gel (Bio-Rad) at 100 volts. In some cases, in order to confirm a particular structure, single stranded DNA were obtained by denaturing where NaOH was added to the nucleic acid samples to a final concentration of 20 mM. The alkalized samples were then incubated at 95° C. for 2 min and cooled immediately on ice before electrophoresis.

The electrophoretic mobility of an oligonucleotide depends on its size, shape and extent of base pairing (Kallenbach et al., J Biomol Struct Dyn 1, 159-68 (1983), which is hereby incorporated by reference in its entirety). Lanes 1-3 of FIG. 2 show the individual single DNA strands (30-mers): Yₒₐ, Y_{ob}, and Y_{oc}. Lanes 4-6 of FIG. 2 represent three possible combinations of Yₒₐ, Y_{ob}, and Y_{oc}, i.e. Yoa with Yob, Yₒₐ, with Y_{OC}, and Y_{ob} with Y_{oc}. One major band appears on the gel, and the mobility is less than the single DNA strands, indicating that one arm of Y-DNA has been formed. Lanes 7-9 of FIG. 2 show the stepwise equal molar mixtures of all three strands, and lane 10 of FIG. 2 shows the all-in-one equal molar mixtures of all three strands. There is no difference in results between stepwise and all-in-one synthesis. Dominant bands, where the mobility has been retarded, suggest that Y-DNA was formed as predicted (See FIG. 1A). The estimated yield of Y-DNA is more than 90%. Other Y-DNA, Y₁, Y₂, Y₃, and Y₄, were similarly built.

### Example 2

### Design, Construction, and Evaluation of Dendrimer-Like DNA Using Y-DNA

For constructing DL-DNA, individual Y-DNAs were ligated specifically to other Y-DNAs, without self-ligation. The ligations were performed with Fast-Link DNA Ligase (Epicentre Technologies, Madison, Mich.). T4 DNA ligase may also be used (Promega Corporation, Madison, Wis.). The reaction scheme is shown in FIGS. 1B and 1C. The nomenclature of DL-DNA is as follows: the core of the dendrimer, Yₒ, is designated as Gₒ, the 0 generation of DL-DNA. After Yₒ is ligated with Y₁, the dendrimer is termed the 1st generation of DL-DNA (G₁), and so on. The n^{th} generation of DL-DNA is noted as Gₙ.

As noted above, each Y-DNA is composed of three single DNA strands (Table 4). These strands are designed so that ligations between Yᵢ and Yⱼ can only occur when i≠j (no self-ligation). In addition, the ligation can only occur in one direction, that is, Yₒ→Y₁→Y₂→Y₃→Y₄. In other words, when Y₀ is ligated to Y₁ with 1:3 stoichiometry, three Y₁ units are linked with one Yₒ forming 1* generation of DL-DNA (G₁). G₁ can then be ligated to six Y₂ units due to the fact that there are 6 arms of Y₁ now (each Y₁ posses two arms), and the resulting product is a second-generation DL-DNM (G₂). A third (G₃), fourth (G₄), and even higher generation DL-DNMs could be synthesized in a similar way.

The first generation DL-DNM was built by ligating Y₀ and Y₁ with 1:3 stoichiometry. The ligation product migrates as a single band, and its mobility is slower than that of its building block, Yₒ. The presence of a single band indicates that a new molecular species with a well-defined stoichiometry has formed. The estimated yield is more than 95%. To further evaluate die structure of the ligation product, it was denatured and examined by gel electrophoresis. There are two major bands for the denatured sample one with the same mobility as the single strand DNA Yₒₐ (30-mer) and one with slower mobility (see arrow, a single stranded 90-mer strand), which is exactly what one would expect according to the assembly scheme. Notice that denaturing G₁ □L-DNM results in two sizes of single strands left: one 30-mer (Y_{1b}) and the other 90-mer ((Y₁ₐ)(Yₒₐ)(Y_{1c})(Y₁ₐ)(Y_{ob})(Y_{1c}), and (Y₁ₐ)(Y_{oc})(Y_{1c})). Taken together, these results indicate that the formation of the 1* generation of DL-DL-DNM is as expected with high yield.

The second, third, and fourth generation DL-DNM were synthesized with, the stepwise approach and evaluated by gel electrophoresis. With each increased generation, the mobility of the ligated product decreased as predicted. The yield and the purity of higher generations (G₃ and G₄) DL-DNM did not decrease, even without purification, indicating, that the stepwise synthesis approach is very robust.

### Example 3

### Atomic Force Imaging of DL-DNA

A 5 ul DNA sample was placed onto the surface of freshly cleaved mica (Ted Pella, Redding, Calif.) functionalized with aminopropyltriethoxysilane (APTES, Aldrich) and allowed to adsorb to the mica surface for approximately 20 minutes. The mica was then rinsed in Milli-Q water and dried with compressed air. Images were taken in air using Tapping mode on a Dimensions 3100 Atomic Force Microscope (Digital Instruments, Santa Barbara, Calif.), and the amplitude setpoint was adjusted to maximize resolution while minimizing the force on the sample. Briefly, the amplitude setpoint was increased until the tip disengaged the surface, and then decreased by 0.1 to 0.2 volts such that the tip was engaged and applying the minimal force onto the sample surface. Images were processed with a flattening filter.

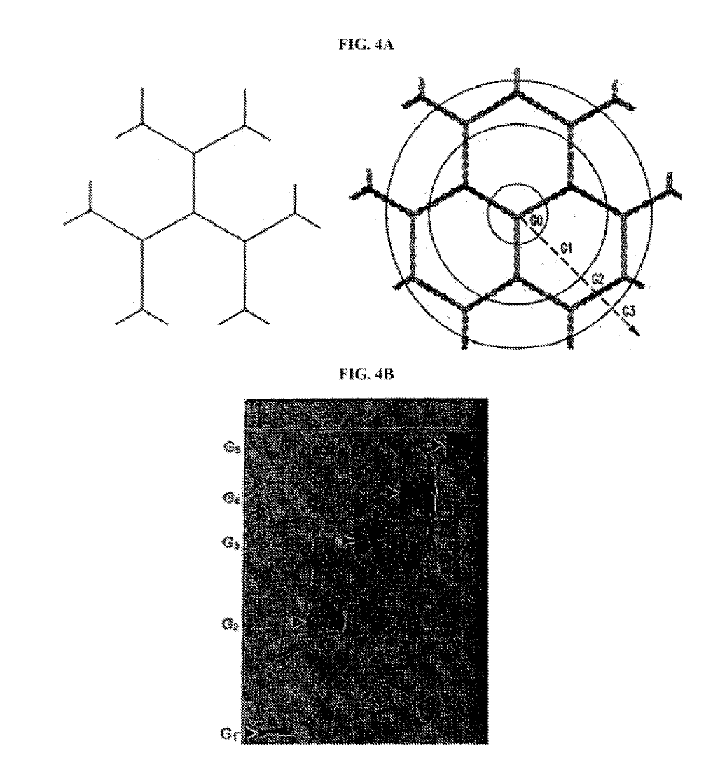

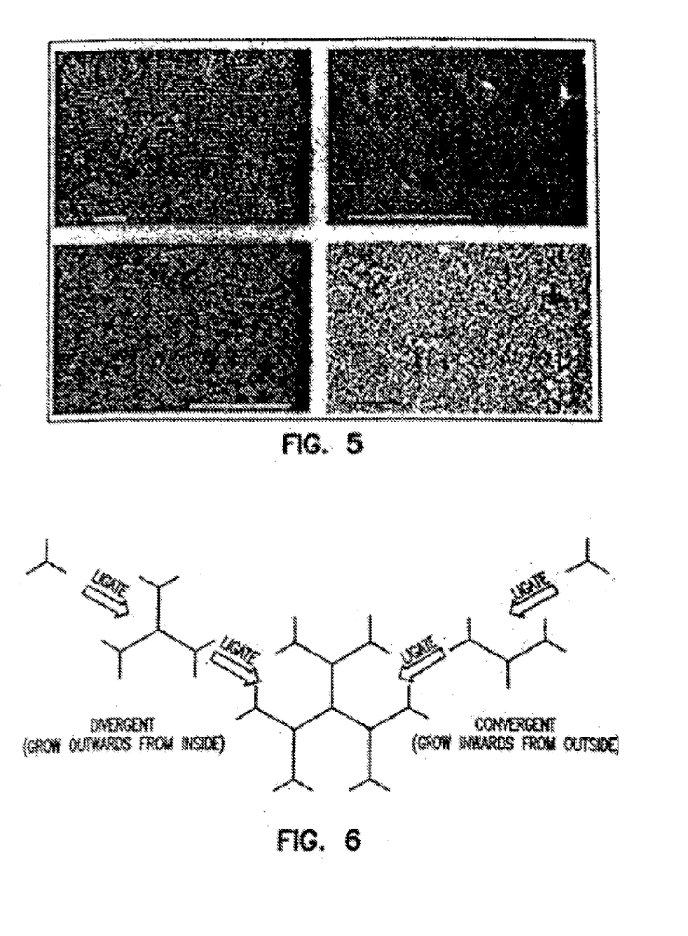

As noted by high-resolution agarose gel electrophoresis, different generations of dendrimers were assembled from basic Y-DNA building blocks. To confirm that the gel-shifted species were indeed DL-DNA molecules, the 4th generation DL-DNM was examined by AFM. FIG. 5 shows clusters of nanoparticles with highly branched DL-DNA molecules. The width of DNA strands was measured to be approximately 9.0 run, consistent with the radius of curvature of the AFM tips. The measured diameter of 4* generation of DL-DNA nanostructure was 71.2±6.7 ma, which was very close to the theoretically calculated value (69.0 nm) considering the relative flexibility of DNA molecules.

### Example 4

### Activation and Conjugation of DL-DNM

Dissolve SMCC in organic solvent, such as dimethylformarnidc (DMF). Re-suspend the amino-modified oligonucleotides in phosphate buffered saline (PBS, pH 7.3-7.5). Mix the DNA with a 40:1 molar excess of SMCC in DMF. Incubate the reaction mixture in the dark at room temperature for 2 hours. Remove free SMCC from activated protein, peptide or oligonucleotide through filtration, for example via Sephadex™ G-25, by simple centrifugation. The excess SMCC can also be removed with a desalting column (Bio-Rad, Hercules, Calif.) using water as the elution buffer. Concentrate the activated oligonucleotides with either Microcon Y-3 (Bedford, Mass.) or freeze-drying.

Slowly mix the above SMCC-activated DNA with an &fold molar excess of NLS peptide (other peptides would be using similar; also see Note 3 for difficult peptides). Adjust the reaction mixture with 10×PBS so that the final solution contains 1×PBS. Incubate the reaction at room temperature with gentle stirring overnight. The crude product can be stored at −20° C. for later processing.

Highly cationic peptides can interact with negatively charged oligonucleotides before a conjugation completes. This interaction can be prevented in a reaction buffer with a high concentration of salt with or without organic solvent (Vives et al. 1997, Tetrahedron Letters, 38:1183-86; Astriab-Fisher et al. 2002; Pharm. Res. 19:744-54). For example, both SMCC functionalized oligonucleotide solution and highly cationic peptide solution are adjusted with 0.5 M KH2P04 (pH 7.S), 4 M KBr and urea to a final concentration of 0.1 M KH2PO₄ (pH 7.9, 0.3 M KBr and 8 M urea (Astriab-Fisher et al. 2002). Acetonitrile (4096, VN) and 0.4 M KCl (Vives et al. 1997) can also be used to facilitate this difficult conjugation. For example, adjusted peptide solution is slowly added to SMCC functionalized oligonucleotide solution with stirring. The reaction mixture is then gently stirred at room temperature for overnight. In addition, highly positively charged peptides can also be selectively conjugated to oligonucleotides through a disulfide bond (Vives et al. 1997; Astriab-Fisher et al. 2002) if the reduction of disilfide in the cytoplasm doesn't interfere with the downstream applications of peptideoligonucleotide.

Since most of the conjugation cannot reach 100% efficiency, the conjugated products need to be purified from unreacted materials using 20% preparative polyacryrnide gel electrophoresis (PAGE). The oligonucleotide-peptide conjugates are separated by conventional PAGE. Cut out the gel slices containing the conjugated products under UV illumination. The gel slices are then crashed with a small syringe and further with shear stress created by vigorous stirring in TE buffer (10 mM Tris-Cl, pH=8.0, 1 mM EDTA). Concentrate the purified and extracted conjugates with a Microcon Y-. Once sequences have been designed, evaluated and characterized, DL-DNA can be synthesized by sequential ligations of Y shaped DNA (Y-DNA) via complementary sticky ends. Note that each sticky-end is designed to be non-palindromic and unique so that selfligation can be totally avoided. The nomenclature of DL-DNM is as follows: the core of the dendrirna, Yo is designated as Go (the oth generation of DL-DNM). After Yo is ligated with YI, the dendrima is termed the generation 1 DL-DNM (G₁), and so on. The n^{th} generation of DL-DNM is noted as Gₙ.

### Example 5

### DL-DNA Assembly: Y-DNA

Each Y-DNA unit is synthesized by annealing 3 single-stranded DNA with a one-pot approach. Dissolve each oligonucleotide strand in an annealing buffer (10 mM Tris pH 8.0, 1 mM EDTA). Combine each oligonucleotide in an equal molar ratio in a microcentrifuge tube. Increase temperature to 95° C. for 5 minutes to denature all oligos. Anneal oligos at 65° C. for 2 minutes. Anneal oligos at 62° C. for 1 minute. Then linearly decrease temperature at a rate of 2° C./minute for 20 minutes. Y-DNA will be formed. If this Y-DNA is used as a core to grow further generation DL-DNM, then this Y-DNA is also called G.-DNA. Store Y-DNA at 4° C.

DL-DNA Assembly: Generation 1

Combine G_{O} and 3 Y-DNA in the appropriate molar ratio (1:3). Add 10% volume of T4 Ligase buffer (5 ul for 50 ul reaction volume) and mix well. Add T4 Ligase based on the enzymatic activity specified on the T4 tube. G₁ will be formed after ligation at room temperature for 16 hours.

DL-DNA Assembly: Generation 2 and Beyond

Combine G₁ and 6 Y-DNA in the appropriate molar ratio (1:6) to form G₂. Repeat ligation steps listed above for G₁ synthesis. Repeat this procedure to generate higher generation DL-DNM. G₂+12 Y-DNA→G₃; G₃+24 Y-DNA→G₄; G₄+48 Y-DNA→G₅.

### Example 6

### DL-DNM Synthesis: Solid Phase Approach

A solid phase approach provides a more robust synthetic route that combines assembly and purification in one step. The products are more pure, and the overall yield is much higher than solution-based synthesis. An extra spacer DNA is needed to attach Y-DNA or DL-DNM onto a solid surface. Sample sequences are listed in Table 4A and 4B, and sample spacer sequences herein below. The scheme of solid phase synthesis of DL-DNA is depicted in FIG. 21.

Spacer 1

| | |
|---|---|
| Biotin-5′-p | (SEQ ID NO: 76 86) |

CCGGATAAGGCGCAGCGGTCGGCTGAATTCAGGGTTCGTGGCAGGCCAG
CACACTTGGAGAC CGAAGCTTACCGGACTCCTAAC-3′
Spacer 2

| | |
|---|---|
| 5′-p-TCA | (SEQ ID NO: 77 87) |

GTTAGGAGTCCGGTAAGCTTCGGTCTCCAAGTGTGCTGGCCTGCCACGA
ACCCTGAATTCAGCCGACCGCTGCGCCTTATCCGG-3′

DL-DNA Solid Phase Assembly: Y-DNA

Without further purification, oligonucleotides, Yₙₐ, Y_{nb} and Y_{nc}, are dissolved in annealing buffer (10 mM Tris, 1 mM EDTA, 50 mM NaCl, pH 8.0) with a final concentration of 0.2 mM. To construct Y-DNA, three oligonucleotide components, Yₙₐ, Y_{nb}, and Y_{nc} (1:1:1 molar ratio) are mixed in sterile Milli-Q water with a final concentration of 40 uM for each oligonucleotide. Hybridizations are performed according to the following procedures: (i) Denaturation at 95° C. for 2 min; (ii) Cooling at 65° C. and incubation for 2 min; (iii) Annealing at 60° C. for 5 min; and (iv) Further annealing at 60° C. for 0.5 min with a continuous temperature decrease at a rate of 1° C. per min The annealing steps were repeated a total of 40 times. The final annealed products were stored at 4° C.

DL-DNA Solid Phase Assembly: Spacer DNA

Two oligonucleotides are synthesized commercially: SP1 and SP2; one of them (SP1) is 5′-biotin modified. Each oligonucleotide is dissolved in a 1×PBS buffer (10 mM phosphate; pH=7.4, 2.7 mM KCl, 137 mM NaCl) with a final concentration of 0.2 mM. The spacer is assembled by hybridizing two oligonucleotide components (1:1 molar ratio) in sterile Milli-Q water with a final concentration of 60 uM for each oligonucleotide. Hybridizations are performed according to the following procedures: (i) Denaturation at 94° C. for 4 min; (ii) Annealing at 80° C. and incubation for 2 min; (iii) Further cooling at 25° C. for 1 hr with a continuous temperature decrease at a rate of 0.5° C. per min. The final annealed products are stored at 4° C.

DL-DNA Solid Phase Assembly: Generation 1

Place 100 ul of avidin coated agarose beads in a 1 ml microcentrifuge tube and then add 1.3 ml of SDS solution to pre-treat the avidin beads. The solution is mixed at 15 rpm rotation for 15-30 min (Step I in Scheme 0). Add 150 ul (8.2 nmole) of spacer DNA into the microcentrifuge tube and then react in a rotary incubator overnight at room temperature. (Step I in Scheme I). The resulting avidin coated beads containing spacer DNA are centrifuged at 2.5 kG and washed with sterile Milli-Q water. (Step I in Scheme I). To grow DL-DNM on beads, individual Y-DNA is ligated specifically to a spacer or other Y-DNA. For example, Gₒ □L-DNM can be obtained by ligating Yₒ to the spacer-modified bead. (Step 11 in Scheme I). Similarly, G₁ is formed by ligating two Y₁ with one G. (Step III in Scheme I). Other higher generations of DL-DNM are constructed using the same strategy. Each ligation reaction solution contains 8.0 mmole of Y-DNA, 2.1 Weiss unit of T4 DNA ligase, ligase buffer (300 mM Tris-HCl in pH 7.8, 100 mM MgCl₂, 100 mM DTT) and 10 mM ATP. (Step 1V-VI in Scheme I). After ligation, the DL-DNM is cleaved off from the solid phase by the restriction enzyme, DDE I. The enzyme solution contained 10 ul of a DDE I and Bovine Serum Albumin (BSA) and restriction buffer D with 60 mM Tris-HCl in pH 7.9, 1.5 M NaCl, 60 mM MgCl₂ and 10 mM DTT. (Step VII in Scheme I).

Testing DNA Delivery Using the DL-NAMs System

Once DL-NAM is synthesized and functionalized subsequently with multi-hctional components, it can be used directly in delivering genes and other nucleic acids (RNAi, for example). The procedures for evaluating cytotoxicity and delivery efficiency have been outlined herein and are known in the art. It is important to note that the DL-NAMs system is a dynamic system in that it is totally modular by design; thus, one can easily “mix and match” different components and “plug and play” to test delivery behaviors. This DL-NAM system provides a platform technology to conjugate a variety of receptors and other targeting molecules, making targeted delivery possible. Our results indicate that cytotoxicity is very low with the DL-NAM system.

A major advantage of this system is the built-in modularity resulting in great flexibility. Both viral and non-viral components can be attached specifically. Pre-made modules will further increase flexibility and make “plug-and-play” possible. Such flexibility is especially useful in studying the complex processes of DNA delivery because, one, little is known quantitatively about intracellular events, and two, one can easily adjust the delivery vector based on the experimental outcomes. In addition, the DL-NAM system is capable of carrying both genes and anti-genes (siRNA), as well as other entities such as enzymes and chemical drugs. A combination of DNA vaccination, gene therapy, antibody/enzyme therapy and si-RNA therapy is thus possible. Examples for modes of delivery of nucleic acids, including for vaccination, are known in the art, as disclosed in U.S. Pat. No. 6,946,448; 6,893,664; 6,821,955; 6,689,757 or 6,562,801.

Furthermore, the size of a DL-NAM vector is designed and constructed at the nanoscale, which is important in intracellular DNA delivery as well as cellular targeting. For example, in one embodiment of the invention, the capability of adding multiple modules, DL-DNA-based DL-NAM provides an advanced platform for constructing an “artificial virus” that utilizes useful viral components to mimic multiple viral functions for DNA delivery with no fear of any viral infection. This DL-DNA-based, nanoscale DL-NAM will play an important role in biomedical and pharmaceutical research.

### Example 7

Construction of the DNA building block X-DNA. The branched DNA sequences (Table 1) were designed and synthesized using commercially available oligonucleotide synthesis. Without further purification, oligonucleotides (Integrated DNA Technologies, Coralville, Iowa) were dissolved in an annealing buffer (10 mM Tris, pH=8.0, 1 mM ethylenediaminetetraacetic acid (EDTA), and 50 mM NaCl) with a final concentration of 50 mM. X-DNA was constructed by mixing four oligonucleotide components (with the same molar ratio) in sterile Milli-Q water with a final concentration of 20 mM for each oligonucleotide. Hybridizations were performed according to the following procedures: (i) denaturation at 95° C. for 2 min (ii) cooling at 65° C. and incubation for 2 min (iii) annealing at 60° C. for 5 min and (iv) further annealing at 60° C. for 0.5 min with a continuous temperature decrease at a rate of 1° C. per min The annealing steps were repeated a total of 40 times. The final annealed products were stored at 4° C. The X₀₁ to X₀₄ were four corresponding single oligonucleotides that formed an X-DNA.

### Example 8

### Screening Assay

This example describes the binding to cells of to a DL-NAM vector comprising a targeting protein (e.g., for illustrative purposes only, such as a chimeric fiber protein) as compared a control DL-NAM not containing a targeting moeity, either in the presence or absence of added soluble said targeting protein.

For these experiments, cells can be selected based on expection to bind with either high efficiency (i.e. receptor-plus cells) or low efficiency (i.e., receptor-minus cells). For example, an epithelial cell line A549 can be used as representative of receptor-plus cells, and the fibroblast cell line HS 68 can be used as representative of receptor-minus cells. Confluent monolayers of either A549 or HS 68 cells can be preincubated at 4.degree. C. with concentrations of soluble fiber protein ranging from 0 to about 10.mu.g/ml. The DL-NAM vector comprising the targeting moeity (T+) or control vector comprising no fiber protein (WT) can be labeled with tritiated thymidine. About 20,000 cpm of [³H]-thymidine labeled DL-NAM or WT vector can be incubated with the cells for about 2 hours at 4° C. The cells were washed three times with cold PBS, and the cell-associated cpm is determined by scintillation counting. Results obtained can be the average of duplicate measurements and are presented for the A549 and HS 68 cell lines, respectively.

Therefore, based on the readings from the scintillation counting receptor-plus and receptor-minus cells will indicate whether a particular moiety or concentration of a particular moiety, as correlated to a particle cell or cell receptor, is effective with high efficiency to effectuate cellular uptake. A DL-NAM can be modified accordingly to increase the number of targeting moieties or to change combinations of different targeting moieties or change targeting moieties altogether.

### Example 9

### Gene Delivery

This example provides results that illustrate that a DL-NAM can be utilized to overcome three currently identified gene delivery barriers at the cellular level: (1) DNA condensation and protection; (2) crossing the plasma membrane via transduction without endocytosis; and (3) nuclear targeting and entry.

Viral peptides were utilized, thus essentially, producing a hybrid viral/non-viral vector (i.e., DL-NAM with viral targeting peptides). For example, Adeno μ (mu) peptide, HIV-1 Rev-NLS or HIV-1 Tat were linked to DL-NAMs. All viral peptides were synthesized with an extra amino acid, Cys, attached at their C-terminal to introduce a free thiol group. In addition, an amine was added to the 5′ end of the DNA with amino modifier C6 to introduce a primary amine group. Conjugation of viral peptides to DL-NAMs was achieved through heterobifunctional crosslinkers such as SMCC that crosslinks between amine and thiol groups. Since each component was designed with only one reactive amine or thiol group, the reactive group was monovalent, and the reaction was terminal. Single stranded DNA was first conjugated with a viral peptide before self-assembled into a viral-Y-DNA FIG. 19. Conjugations of each viral peptide to single stranded DNA were all successful. Functions of each viral-Y-DNA was tested and confirmed either separately or combinatorially. For example, DNA condensation via Tat-Y-DNA was evident on gel electrophoretic retardation assay, which showed that Tat-Y-DNA had higher mobility as compared to condensed DNA without Tat.

A plasmid DNA (pVax/LacZ) coding for the LacZ gene was used as a reporter gene. When tagged with fluorescent dye FAM, the fate of the hybrid vectors was followed in real time. LacZ gene expression was quantified and compared with various lipid-based vectors. The results showed that the DL-NAMs successfully crossed the cell plasma membrane by viral peptides. In addition, gene expression utilizing DL-NAMs was substantially higher as compared to controls.

As noted above, a major advantage of this hybrid system is the built-in modularity. Both viral and non-viral components can be attached specifically and independently. Preformed modules will further increase the flexibility and make “plug-n-play” a reality using DL-NAMs as a platform. Moreover, DL-NAM conjugation (e.g., by varying concentrations of DL-NAM, or polynucleotides to target) can be easily altered based on cell culture or animal model experiments. Key advantages of the DL-NAM are its nanoscale size (allowing intracellular delivery), modularity (allowing drugs, antibody, enzymes, nucleic acids to be incorporated in a controlled fashion), and a nucleic acid backbone (which will eventually be degraded into its natural monomers, thus biodegradable and biocompatible).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed:

1. A method of treating a disease or condition comprising:
administering an effective amount of a delivery vector comprising:
a first, a second, and a third polynucleotide, wherein at least a portion of the first polynucleotide is complementary to at least a portion of the second polynucleotide, wherein at least a portion of the first polynucleotide is complementary to at least a portion of the third polynucleotide, wherein at least a portion of the second polynucleotide is complementary to at least a portion of the third polynucleotide, and wherein the first, second, and third polynucleotides are associated together to form a trimer and
at least one of the first, second, and third polynucleotides is linked to at least one therapeutic agent.

2. The method of claim 1, wherein the therapeutic agent comprises a drug, nucleic acid molecule, small organic molecule, or small inorganic molecule.

3. The method of claim 2, wherein the nucleic acid molecule is a DNA vaccine, a therapeutic gene, an RNAi, an siRNA, an aptamer, a receptor ligand, or an antisense molecule.

4. The method of claim 2, wherein the drug is an anti-cancer substance, analgesic, opioid, anti-AIDS substance, immunosuppressants, anti-viral agent, enzyme inhibitor, neurotoxin, hypnotic, anti-histamine, lubricant, tranquilizer, anti-convulsant, muscle relaxant, anti-Parkinson agent, anti-spasmodic, muscle contractant, channel blocker, miotic, anti-cholinergic, anti-glaucoma compound, anti-parasite compound, anti-protozoal compound, anti-fungal compound, modulators of cell-extracellular matrix interaction, cell growth inhibitor, anti-adhesion molecule, vasodilating agent, inhibitor of DNA, RNA or protein synthesis, anti-hypertensive, anti-pyretic, steroidal anti-inflammatory agent, non-steroidal anti-inflammatory agent, anti-angiogenic factor, anti-secretory factor, anticoagulant, antithrombotic agent, local anesthetic, ophthalmic, prostaglandin, or neurotransmitter.

5. The method of claim 1, wherein the therapeutic agent comprises a bioactive protein or peptide.

6. The method of claim 5, wherein the bioactive protein or peptide comprises a cell modulating peptide, a chemotactic peptide, an anticoagulant peptide, an antithrombotic peptide, an anti-tumor peptide, an anti-infectious peptide, a growth potentiating peptide, or an anti-inflammatory peptide.

7. The method of claim 5, wherein the bioactive protein or peptide comprises an antibody, enzyme, steroid, growth hormone, growth hormone-releasing hormone, gonadotropin-releasing hormone, agonist, or antagonist, somatostatin, gonadotropin, luteinizing hormone, follicle-stimulating hormone, peptide T, thyrocalcitonin, parathyroid hormone, glucagon, vasopressin, oxytocin, angiotensin I and II, bradykinin, kallidin, adrenocorticotropic hormone, thyroid stimulating hormone, insulin, glucagon or any analogue thereof.

8. The method of claim 5, wherein the therapeutic agent is insulin.

9. The method of claim 5, wherein the therapeutic agent is an antigen selected from the group consisting of MMR (mumps, measles and rubella) vaccine, typhoid vaccine, hepatitis A vaccine, hepatitis B vaccine, herpes simplex virus, bacterial toxoids, cholera toxin B-subunit, influenza vaccine virus, bordetela pertussis virus, vaccinia virus, adenovirus, canary pox, polio vaccine virus, plasmodium falciparum, bacillus calmette geurin (BCG), klebsiella pneumoniae, HIV envelope glycoproteine.

10. The method of claim 5, wherein the therapeutic agent is a cytokine

11. The method of claim 1, wherein the therapeutic agent is linked directly or indirectly to the delivery vector.

12. The method of claim 1, wherein at least one of the first, second, and third polynucleotides is linked to at least one peptide moiety.

13. The method of claim 12, wherein the peptide moiety comprises an adenovirus core peptide, a synthetic peptide, an influenza virus HA2 peptide, a simian immunodeficiency virus gp32 peptide, an SV40 T-Ag peptide, a VP22 peptide, a Tat peptide, a Rev peptide, DNA condensing peptide, DNA protection peptide, endosomal targeting peptide, membrane fusion peptide, nuclear localization signaling peptide, a protein transduction domain peptide or any combination thereof.

14. The method of claim 12, wherein the therapeutic agent is linked directly or indirectly to the at least one peptide moiety.

15. The method of claim 1, wherein at least one of the first, second, and third polynucleotides is linked to a detectable label.

16. The method of claim 15, wherein the therapeutic agent is linked directly or indirectly to the at least one detectable label.

17. The method of claim 15, wherein the detectable label comprises a chromophore, fluorescent moiety, enzyme, antigen, heavy metal, magnetic probe, dye, phosphorescent group, radioactive material, chemiluminescent moiety, scattering or fluorescent nanoparticle, Raman signal generating moiety, quantum dot, or electrochemical detection moiety.

18. The method of claim 1, wherein the therapeutic agent is a plasmid or viral vector.

19. The method of claim 18, wherein the plasmid or viral vector encodes a therapeutic gene.

20. The method of claim 1, wherein the delivery vector is linked to at least two therapeutic agents.

21. The method of claim 1, wherein at least one of the first, second, and third polynucleotides is functionalized with a lipid.
